(19) United States
(12) Patent Application Publication
(10) Pub. No.: US 2024/0100162 A1
(43) Pub. Date: Mar. 28, 2024

(54) MAGE-B2-SPECIFIC T-CELL RECEPTORS

(71) Applicant: AMGEN INC., Thousand Oaks, CA (US)

(72) Inventors: Sungeun KIM, San Mateo, CA (US); Yan ZHENG, Thousand Oaks, CA (US); Dhanashri S. BAGAL, Thousand Oaks, CA (US); Lili YUE, Thousand Oaks, CA (US)

(73) Assignee: AMGEN INC., Thousand Oaks, CA (US)

(21) Appl. No.: 18/038,283

(22) PCT Filed: Dec. 21, 2021
(86) PCT No.: PCT/US21/64504

§ 371 Date: May 23, 2023

Related U.S. Application Data
(60) Provisional application No. 63/129,447, filed on Dec. 22, 2020.

(51) Int. Cl. A61K 39/00; A61P 35/00; C07K 14/54; C07K 14/725; C12N 5/0783; C12N 15/86; C12Q 1/6869; C12Q 1/6886; G01N 33/574

(52) U.S. Cl. CPC A61K 39/464486; A61K 39/4611; A61K 39/4632; A61P 35/00; C07K 14/5434; C07K 14/7051; C12N 5/0636; C12N 15/86; C12Q 1/6869; C12Q 1/6886; G01N 33/57484; C12N 2740/15043; C12Q 2600/106; C12Q 2600/156

(57) ABSTRACT

Provided herein are T-cell receptors (TCRs) that when expressed recombinantly on the surface of a T cell are able to recognize the MAGE-B2-derived peptide GVYDGEEHSV (SEQ ID NO: 1) when presented by HLA-A*02:01 sufficiently to activate the recombinant T cell. Certain TCRs provided herein also are able to recognize the MAGE-A4-derived peptide GVYDGREHTV (SEQ ID NO:2) sufficiently to activate the recombinant T cell. Importantly, exemplary TCRs provided herein were thoroughly screened for lack of cross-reactivity with similar peptides that may be presented by normal cells or tissue and for alloreactivity.

This application claims the benefit of U.S. Provisional Application No. 63/129,447, filed Dec. 22, 2020, which is hereby incorporated by reference in its entirety and for all purposes as if fully set forth herein.

### FIELD OF DISCLOSURE

The present invention relates to T-cell receptors that when expressed recombinantly on the surface of a T cell are able to recognize peptides sufficiently to activate the recombinant T cell.

### SEQUENCE LISTING

This application contains, as a separate part of the disclosure, a sequence list in computer-readable form (Filename: A-2668-WO-PCT_ST25.txt, created 11/2/2021, which is 113 KB in size), and which is incorporated by reference in its entirety.

### BACKGROUND

Adoptive T cell therapies provide tremendous opportunities to treat cancer. Chimeric antigen receptor (CAR)-T cell therapy is an approved adoptive T cell therapy for hematological malignancy but has a limited range of targets due to its recognition to only cell surface antigens constituting ˜25% of the genome. Unlike CAR-T cells, TCR-T cells engineered to express the T cell receptors (TCR) specific to tumor antigens can exploit a broader range of targets for multiple cancer indications because TCR-T cells can recognize the peptide-MHC complexes (pMHC) derived from intracellular proteins constituting ˜75% of the genome. Intracellular proteins are processed and presented by major histocompatibility complex (MHC) as pMHC complexes.

Cancer-testis antigens (CTA) are attractive targets for cancer immunotherapy including TCR-T cell therapy due to their restricted expression in germ cells and aberrant reactivation in various cancers, and their immunogenic properties. Germ cells such as testis (immune-privileged sites) do not usually express HLA class I/II molecules, allowing them to evade attack from the immune system. MAGE-B2 and MAGE-A4 are members of the melanoma antigen (MAGE) gene family, most of which are classified as intracellular cancer-testis antigens including MAGE-B2 and MAGE-A4. Recent studies have suggested that MAGEs assemble with E3 RING ubiquitin ligases, act as regulators of ubiquitination, play roles in cell proliferation and oncogenic activity, and regulate the cellular stress response. However, the functions of most MAGE genes including MAGE-B2 and MAGE-A4 are not fully understood.

While TCR-T cells are shown to be very potent and sensitive modality for tumor-specific peptide-MHC targets, a TCR can recognize multiple peptides. DNA rearrangement required for TCR formation generates a certain number of T cells that recognize self-antigens. During early T cell development, self-reactive T cells are negatively selected and eliminated in the medulla of the thymus through a promiscuous expression of a wide range of self-antigens in medullary thymic epithelial cells. This negative selection in the thymus functions as the major mechanism of central tolerance and shapes the T cell repertoire to avoid autoimmunity. TCRs that are engineered to increase their affinity for certain pMHC or to introduce cross-reactivity to multiple pMHC do not have the benefit of the negative selection that occurs in the thymus. It is noteworthy that affinity-enhanced MAGE-A3 TCR-T cells led to fatal toxicity due to cross-reactivity to Titin expressed in cardiac muscles (Cameron et al., Sci Transl Med. 2013 5 (197)).

### SUMMARY

Identification of TCR sequences recognizing tumor-specific antigens has been shown to be very challenging in the field particularly due to rarity of tumor-specific T cells in patient blood, difficulty in expanding a very small number of tumor-specific T cell clones ex vivo, and potential exhaustion or suppression of tumor-specific T cells in tumor-infiltrating lymphocytes (TILs). Despite these challenges, provided herein are TCR sequences specific to MAGE-B2 peptide-MHC (GVYDGEEHSV/HLA-A*02:01) and MAGE-A4 peptide-MHC (GVYDGREHTV/HLA-A*02:01) identified by using healthy donor blood and an ex vivo stimulation method. As demonstrated in the Examples herein, the exemplary TCR-T cells recognizing the tumor-specific MAGE-B2 pMHC, and in some embodiments MAGE-A4, pMHC can be highly potent therapeutics for the treatment of MAGE-B2+/HLA-A*02:01+ and/or MAGE-A4+/HLA-A*02:01+ tumors by exerting cytotoxicity and producing cytokines. These TCR-T cell therapies will be a significant treatment option for a wide variety of cancer indications.

TCR-T cells are the most potent and sensitive modality in vitro for pMHC targets. The TCR-T cells provided herein display high potency against even very low target-expressing cells. This high potency of TCR-T cells comes from the complex of the transduced TCR and endogenous CD3 subunits. In addition, to enhance in vivo efficacy, exemplary TCR-T cells comprise an activation-dependent IL12 payload that is incorporated into a TCR-T construct where IL12 expression is regulated by TCR activation under a composite promoter containing six NFAT (nuclear factor of activated T cells) response elements linked to a minimal IL-2 promoter. Therefore, when TCR-T-IL12 cells encounter tumor antigens, the IL12 is produced. As shown in the mouse studies provided in the Examples, IL12 payload enhanced the efficacy of adoptive T cell therapy in vivo and therefore could decrease potential clinical dose (by 10-100×).

In a first aspect, the present invention is an expression vector comprising a nucleic acid sequence encoding a T-cell receptor (TCR) alpha chain and a TCR beta chain, wherein the TCR alpha chain and TCR beta chain are selected from the group consisting of:
a. a TCR alpha chain comprising an amino acid sequence set forth in SEQ ID NO: 13 and a TCR beta chain comprising an amino acid sequence set forth in SEQ ID NO:24;
b. a TCR alpha chain comprising an amino acid sequence set forth in SEQ ID NO: 14 and a TCR beta chain comprising an amino acid sequence set forth in SEQ ID NO:25;
c. a TCR alpha chain comprising an amino acid sequence set forth in SEQ ID NO: 15 and a TCR beta chain comprising an amino acid sequence set forth in SEQ ID NO:26;
d. a TCR alpha chain comprising an amino acid sequence set forth in SEQ ID NO: 16 and a TCR beta chain comprising an amino acid sequence set forth in SEQ ID NO:27;
e. a TCR alpha chain comprising an amino acid sequence set forth in SEQ ID NO: 17 and a TCR beta chain comprising an amino acid sequence set forth in SEQ ID NO:28;
f. a TCR alpha chain comprising an amino acid sequence set forth in SEQ ID NO: 18 and a TCR beta chain comprising an amino acid sequence set forth in SEQ ID NO:29;
g. a TCR alpha chain comprising an amino acid sequence set forth in SEQ ID NO: 19 and a TCR beta chain comprising an amino acid sequence set forth in SEQ ID NO:30;
h. a TCR alpha chain comprising an amino acid sequence set forth in SEQ ID NO:20 and a TCR beta chain comprising an amino acid sequence set forth in SEQ ID NO:31;
i. a TCR alpha chain comprising an amino acid sequence set forth in SEQ ID NO:21 and a TCR beta chain comprising an amino acid sequence set forth in SEQ ID NO:32;
j. a TCR alpha chain comprising an amino acid sequence set forth in SEQ ID NO:22 and a TCR beta chain comprising an amino acid sequence set forth in SEQ ID NO:33; and
k. a TCR alpha chain comprising an amino acid sequence set forth in SEQ ID NO:23 and a TCR beta chain comprising an amino acid sequence set forth in SEQ ID NO:34.

Any expression vector of the first aspect may further comprise a nucleic acid encoding interleukin-12 (IL-12) or a functional variant thereof and may be a viral vector such as a retroviral or lentiviral vector.

In certain embodiments of the first aspect, the expression vector encodes a TCR alpha chain having a CDR3 region amino acid sequence as set forth in SEQ ID NO:13 and the TCR beta chain a CDR3 region amino acid sequence as set forth in SEQ ID NO:24. In preferred embodiments, the mature TCR alpha chain comprises an amino acid sequence set forth in SEQ ID NO:35 and the mature TCR beta chain comprises an amino acid sequence set forth in SEQ ID NO:46. The expression vector may encode the full-length TCR alpha chain comprising the amino acid sequence set forth in SEQ ID NO:57 and the full-length TCR beta chain comprising the amino acid sequence set forth in SEQ ID NO:68.

In certain embodiments of the first aspect, the expression vector encodes a TCR alpha chain having a CDR3 region amino acid sequence as set forth in SEQ ID NO:14 and the TCR beta chain a CDR3 region amino acid sequence as set forth in SEQ ID NO:25. In preferred embodiments, the mature TCR alpha chain comprises an amino acid sequence set forth in SEQ ID NO:36 and the mature TCR beta chain comprises an amino acid sequence set forth in SEQ ID NO:47. The expression vector may encode the full-length TCR alpha chain comprising the amino acid sequence set forth in SEQ ID NO:58 and the full-length TCR beta chain comprising the amino acid sequence set forth in SEQ ID NO:69.

In certain embodiments of the first aspect, the expression vector encodes a TCR alpha chain having a CDR3 region amino acid sequence as set forth in SEQ ID NO:15 and the TCR beta chain a CDR3 region amino acid sequence as set forth in SEQ ID NO:26. In preferred embodiments, the mature TCR alpha chain comprises an amino acid sequence set forth in SEQ ID NO:37 and the mature TCR beta chain comprises an amino acid sequence set forth in SEQ ID NO:48. The expression vector may encode the full-length TCR alpha chain comprising the amino acid sequence set forth in SEQ ID NO:59 and the full-length TCR beta chain comprising the amino acid sequence set forth in SEQ ID NO:70.

In certain embodiments of the first aspect, the expression vector encodes a TCR alpha chain having a CDR3 region amino acid sequence as set forth in SEQ ID NO: 16 and the TCR beta chain a CDR3 region amino acid sequence as set forth in SEQ ID NO:27. In preferred embodiments, the mature TCR alpha chain comprises an amino acid sequence set forth in SEQ ID NO:38 and the mature TCR beta chain comprises an amino acid sequence set forth in SEQ ID NO:49. The expression vector may encode the full-length TCR alpha chain comprising the amino acid sequence set forth in SEQ ID NO:60 and the full-length TCR beta chain comprising the amino acid sequence set forth in SEQ ID NO:71.

In certain embodiments of the first aspect, the expression vector encodes a TCR alpha chain having a CDR3 region amino acid sequence as set forth in SEQ ID NO:17 and the TCR beta chain a CDR3 region amino acid sequence as set forth in SEQ ID NO:28. In preferred embodiments, the mature TCR alpha chain comprises an amino acid sequence set forth in SEQ ID NO:39 and the mature TCR beta chain comprises an amino acid sequence set forth in SEQ ID NO:50. The expression vector may encode the full-length TCR alpha chain comprising the amino acid sequence set forth in SEQ ID NO:61 and the full-length TCR beta chain comprising the amino acid sequence set forth in SEQ ID NO:72.

In certain embodiments of the first aspect, the expression vector encodes a TCR alpha chain having a CDR3 region amino acid sequence as set forth in SEQ ID NO:18 and the TCR beta chain a CDR3 region amino acid sequence as set forth in SEQ ID NO:29. In preferred embodiments, the mature TCR alpha chain comprises an amino acid sequence set forth in SEQ ID NO:40 and the mature TCR beta chain comprises an amino acid sequence set forth in SEQ ID NO:51. The expression vector may encode the full-length TCR alpha chain comprising the amino acid sequence set forth in SEQ ID NO:62 and the full-length TCR beta chain comprising the amino acid sequence set forth in SEQ ID NO:73.

In certain embodiments of the first aspect, the expression vector encodes a TCR alpha chain having a CDR3 region amino acid sequence as set forth in SEQ ID NO:19 and the TCR beta chain a CDR3 region amino acid sequence as set forth in SEQ ID NO:30. In preferred embodiments, the mature TCR alpha chain comprises an amino acid sequence set forth in SEQ ID NO:41 and the mature TCR beta chain comprises an amino acid sequence set forth in SEQ ID NO:52. The expression vector may encode the full-length TCR alpha chain comprising the amino acid sequence set forth in SEQ ID NO:63 and the full-length TCR beta chain comprising the amino acid sequence set forth in SEQ ID NO:74.

In certain embodiments of the first aspect, the expression vector encodes a TCR alpha chain having a CDR3 region amino acid sequence as set forth in SEQ ID NO:20 and the TCR beta chain a CDR3 region amino acid sequence as set forth in SEQ ID NO:31. In preferred embodiments, the mature TCR alpha chain comprises an amino acid sequence set forth in SEQ ID NO:42 and the mature TCR beta chain comprises an amino acid sequence set forth in SEQ ID NO:53. The expression vector may encode the full-length TCR alpha chain comprising the amino acid sequence set forth in SEQ ID NO:64 and the full-length TCR beta chain comprising the amino acid sequence set forth in SEQ ID NO:75.

In certain embodiments of the first aspect, the expression vector encodes a TCR alpha chain having a CDR3 region amino acid sequence as set forth in SEQ ID NO:21 and the TCR beta chain a CDR3 region amino acid sequence as set forth in SEQ ID NO:32. In preferred embodiments, the mature TCR alpha chain comprises an amino acid sequence set forth in SEQ ID NO:43 and the mature TCR beta chain comprises an amino acid sequence set forth in SEQ ID NO:54. The expression vector may encode the full-length TCR alpha chain comprising the amino acid sequence set forth in SEQ ID NO:65 and the full-length TCR beta chain comprising the amino acid sequence set forth in SEQ ID NO:76.

In certain embodiments of the first aspect, the expression vector encodes a TCR alpha chain having a CDR3 region amino acid sequence as set forth in SEQ ID NO:22 and the TCR beta chain a CDR3 region amino acid sequence as set forth in SEQ ID NO:33. In preferred embodiments, the mature TCR alpha chain comprises an amino acid sequence set forth in SEQ ID NO:44 and the mature TCR beta chain comprises an amino acid sequence set forth in SEQ ID NO:55. The expression vector may encode the full-length TCR alpha chain comprising the amino acid sequence set forth in SEQ ID NO:66 and the full-length TCR beta chain comprising the amino acid sequence set forth in SEQ ID NO:77.

In certain embodiments of the first aspect, the expression vector encodes a TCR alpha chain having a CDR3 region amino acid sequence as set forth in SEQ ID NO:23 and the TCR beta chain a CDR3 region amino acid sequence as set forth in SEQ ID NO:34. In preferred embodiments, the mature TCR alpha chain comprises an amino acid sequence set forth in SEQ ID NO:45 and the mature TCR beta chain comprises an amino acid sequence set forth in SEQ ID NO:56. The expression vector may encode the full-length TCR alpha chain comprising the amino acid sequence set forth in SEQ ID NO:67 and the full-length TCR beta chain comprising the amino acid sequence set forth in SEQ ID NO:78.

In a second aspect, is a cell expressing a recombinant T-cell receptor (TCR), said TCR comprising:
a. a TCR alpha chain CDR3 region comprising an amino acid sequence set forth in SEQ ID NO:13 and a TCR beta chain CDR3 region comprising an amino acid sequence set forth in SEQ ID NO:24;
b. a TCR alpha chain CDR3 region comprising an amino acid sequence set forth in SEQ ID NO:14 and a TCR beta chain CDR3 region comprising an amino acid sequence set forth in SEQ ID NO:25;
c. a TCR alpha chain CDR3 region comprising an amino acid sequence set forth in SEQ ID NO: 15 and a TCR beta chain CDR3 region comprising an amino acid sequence set forth in SEQ ID NO:26;
d. a TCR alpha chain CDR3 region comprising an amino acid sequence set forth in SEQ ID NO: 16 and a TCR beta chain CDR3 region comprising an amino acid sequence set forth in SEQ ID NO:27;
e. a TCR alpha chain CDR3 region comprising an amino acid sequence set forth in SEQ ID NO: 17 and a TCR beta chain CDR3 region comprising an amino acid sequence set forth in SEQ ID NO:28;
f. a TCR alpha chain CDR3 region comprising an amino acid sequence set forth in SEQ ID NO:18 and a TCR beta chain CDR3 region comprising an amino acid sequence set forth in SEQ ID NO:29;
g. a TCR alpha chain CDR3 region comprising an amino acid sequence set forth in SEQ ID NO: 19 and a TCR beta chain CDR3 region comprising an amino acid sequence set forth in SEQ ID NO: 30;
h. a TCR alpha chain CDR3 region comprising an amino acid sequence set forth in SEQ ID NO:20 and a TCR beta chain CDR3 region comprising an amino acid sequence set forth in SEQ ID NO:31;
i. a TCR alpha chain CDR3 region comprising an amino acid sequence set forth in SEQ ID NO:21 and a TCR beta chain CDR3 region comprising an amino acid sequence set forth in SEQ ID NO: 32;
j. a TCR alpha chain CDR3 region comprising an amino acid sequence set forth in SEQ ID NO:22 and a TCR beta chain CDR3 region comprising an amino acid sequence set forth in SEQ ID NO:33; or
k. a TCR alpha chain CDR3 region comprising an amino acid sequence set forth in SEQ ID NO:23 and a TCR beta chain CDR3 region comprising an amino acid sequence set forth in SEQ ID NO: 34.

In preferred embodiments of the second aspect, the cell recombinantly expresses a TCR comprising:
a. a TCR alpha chain comprising an amino acid sequence set forth in SEQ ID NO:35 and a TCR beta chain comprising an amino acid sequence set forth in SEQ ID NO:46;
b. a TCR alpha chain comprising an amino acid sequence set forth in SEQ ID NO:36 and a TCR beta chain comprising an amino acid sequence set forth in SEQ ID NO:47;
c. a TCR alpha chain comprising an amino acid sequence set forth in SEQ ID NO:37 and a TCR beta chain comprising an amino acid sequence set forth in SEQ ID NO:48;
d. a TCR alpha chain comprising an amino acid sequence set forth in SEQ ID NO:38 and a TCR beta chain comprising an amino acid sequence set forth in SEQ ID NO:49;
e. a TCR alpha chain comprising an amino acid sequence set forth in SEQ ID NO:39 and a TCR beta chain comprising an amino acid sequence set forth in SEQ ID NO:50;
f. a TCR alpha chain comprising an amino acid sequence set forth in SEQ ID NO:40 and a TCR beta chain comprising an amino acid sequence set forth in SEQ ID NO:51;
g. a TCR alpha chain comprising an amino acid sequence set forth in SEQ ID NO:41 and a TCR beta chain comprising an amino acid sequence set forth in SEQ ID NO:52;
h. a TCR alpha chain comprising an amino acid sequence set forth in SEQ ID NO:42 and a TCR beta chain comprising an amino acid sequence set forth in SEQ ID NO:53;
i. a TCR alpha chain comprising an amino acid sequence set forth in SEQ ID NO:43 and a TCR beta chain comprising an amino acid sequence set forth in SEQ ID NO:54;
j. a TCR alpha chain comprising an amino acid sequence set forth in SEQ ID NO:44 and a TCR beta chain comprising an amino acid sequence set forth in SEQ ID NO:55; or
k. a TCR alpha chain comprising an amino acid sequence set forth in SEQ ID NO:45 and a TCR beta chain comprising an amino acid sequence set forth in SEQ ID NO:56.

The cell of the second aspect further may express a recombinant IL-12 or functional variant thereof.

In certain embodiments of the second aspect, the cell comprises one or more expression vectors of the first aspect.

The cell may be a T cell and, when the TCR binds the peptide of SEQ ID NO:1 or SEQ ID NO:2 in the context of HLA-A*02:01, the binding leads to activation of IFNγ, TNFα, IL-12, or granzyme B production by the cell.

In a third aspect of the invention, a pharmaceutical composition comprises a therapeutically effective amount of a cell of the second aspect or an expression vector of the first aspect.

In a fourth aspect, the invention provides a method of making a cell of the second aspect or a pharmaceutical composition of the third aspect, comprising introducing into a cell an expression vector comprising a nucleic acid sequence encoding a TCR alpha chain and a TCR beta chain, wherein the TCR alpha chain and TCR beta chain are selected from the group consisting of:
a. a TCR alpha chain comprising a CDR3 region having an amino acid sequence set forth in SEQ ID NO:13 and a TCR beta chain comprising a CDR3 region having an amino acid sequence set forth in SEQ ID NO:24;
b. a TCR alpha chain comprising a CDR3 region having an amino acid sequence set forth in SEQ ID NO:14 and a TCR beta chain comprising a CDR3 region having an amino acid sequence set forth in SEQ ID NO:25;
c. a TCR alpha chain comprising a CDR3 region having an amino acid sequence set forth in SEQ ID NO:15 and a TCR beta chain comprising a CDR3 region having an amino acid sequence set forth in SEQ ID NO:26;
d. a TCR alpha chain comprising a CDR3 region having an amino acid sequence set forth in SEQ ID NO:16 and a TCR beta chain comprising a CDR3 region having an amino acid sequence set forth in SEQ ID NO:27;
e. a TCR alpha chain comprising a CDR3 region having an amino acid sequence set forth in SEQ ID NO:17 and a TCR beta chain comprising a CDR3 region having an amino acid sequence set forth in SEQ ID NO:28;
f. a TCR alpha chain comprising a CDR3 region having an amino acid sequence set forth in SEQ ID NO:18 and a TCR beta chain comprising a CDR3 region having an amino acid sequence set forth in SEQ ID NO:29;
g. a TCR alpha chain comprising a CDR3 region having an amino acid sequence set forth in SEQ ID NO:19 and a TCR beta chain comprising a CDR3 region having an amino acid sequence set forth in SEQ ID NO:30;
h. a TCR alpha chain comprising a CDR3 region having an amino acid sequence set forth in SEQ ID NO:20 and a TCR beta chain comprising a CDR3 region having an amino acid sequence set forth in SEQ ID NO:31;
i. a TCR alpha chain comprising a CDR3 region having an amino acid sequence set forth in SEQ ID NO:21 and a TCR beta chain comprising a CDR3 region having an amino acid sequence set forth in SEQ ID NO:32;
j. a TCR alpha chain comprising a CDR3 region having an amino acid sequence set forth in SEQ ID NO:22 and a TCR beta chain comprising a CDR3 region having an amino acid sequence set forth in SEQ ID NO:33; or
k. a TCR alpha chain comprising a CDR3 region having an amino acid sequence set forth in SEQ ID NO:23 and a TCR beta chain comprising a CDR3 region having an amino acid sequence set forth in SEQ ID NO:34.

In preferred embodiments of the fourth aspect, the TCR alpha chain and TCR beta chain are selected from the group consisting of:
a. a TCR alpha chain comprising an amino acid sequence set forth in SEQ ID NO:35 and a TCR beta chain comprising an amino acid sequence set forth in SEQ ID NO:46;
b. a TCR alpha chain comprising an amino acid sequence set forth in SEQ ID NO:36 and a TCR beta chain comprising an amino acid sequence set forth in SEQ ID NO:47;
c. a TCR alpha chain comprising an amino acid sequence set forth in SEQ ID NO:37 and a TCR beta chain comprising an amino acid sequence set forth in SEQ ID NO:48;
d. a TCR alpha chain comprising an amino acid sequence set forth in SEQ ID NO:38 and a TCR beta chain comprising an amino acid sequence set forth in SEQ ID NO:49;
e. a TCR alpha chain comprising an amino acid sequence set forth in SEQ ID NO:39 and a TCR beta chain comprising an amino acid sequence set forth in SEQ ID NO:50;
f. a TCR alpha chain comprising an amino acid sequence set forth in SEQ ID NO:40 and a TCR beta chain comprising an amino acid sequence set forth in SEQ ID NO:51;
g. a TCR alpha chain comprising an amino acid sequence set forth in SEQ ID NO:41 and a TCR beta chain comprising an amino acid sequence set forth in SEQ ID NO:52;
h. a TCR alpha chain comprising an amino acid sequence set forth in SEQ ID NO:42 and a TCR beta chain comprising an amino acid sequence set forth in SEQ ID NO:53;
i. a TCR alpha chain comprising an amino acid sequence set forth in SEQ ID NO:43 and a TCR beta chain comprising an amino acid sequence set forth in SEQ ID NO:54;
j. a TCR alpha chain comprising an amino acid sequence set forth in SEQ ID NO:44 and a TCR beta chain comprising an amino acid sequence set forth in SEQ ID NO:55; and
k. a TCR alpha chain comprising an amino acid sequence set forth in SEQ ID NO:45 and a TCR beta chain comprising an amino acid sequence set forth in SEQ ID NO:56.

In certain embodiments of the fourth aspect, a nucleic acid sequence encoding IL-12 or a functional variant thereof is also introduced into the cell and may be on an expression vector encoding the alpha chain and/or beta chain or may be encoded on a separate vector.

The cell made by a method of the fourth aspect may be a primary T cell isolated from a cancer patient.

In a fifth aspect, the invention provides methods of treating a MAGE-B2 or MAGE-A4 expressing cancer, said method comprising administering to a cancer patient a therapeutically effective amount of a cell of the second aspect, a pharmaceutical composition of the third aspect, or of a cell made by the method of the fourth aspect. In certain embodiments of the fifth aspect, the patient is tested prior to administration to determine the presence of a cancer expressing MAGE-B2 or MAGE-A4. The test may detect a MAGE-B2- or MAGE-A4-encoding nucleic acid, a MAGE-B2 or MAGE-A4 protein, or a MAGE-B2-derived or MAGE-A4-derived peptide. In preferred embodiments, the patient is identified as carrying the HLA-A*02:01 allele.

### BRIEF DESCRIPTION OF THE DRAWINGS

FIG. 1. (A) MAGE-A4 and MAGE-B2 mRNA expression in a variety of cancers (TCGA and internal RNA-seq data). (B) MAGE-A4 and MAGE-B2 mRNA expression in human normal tissues (Amgen Body map RNA-seq data). (C) MAGE-A4 immunohistochemistry (IHC) by OT11F9 monoclonal Ab shows that within a tumor of NSCLC-squamous, MAGE-A4 protein is expressed in the majority of tumor cells. The representative IHC stains of NSCLC-squamous tumors show 100% MAGE-A4 positive tumor cells and 3+ intense staining.

FIG. 2. (A) Mass spectrometry (MS) data (Immatics) demonstrates MAGE-B2 peptide-HLA-A*02:01 is expressed in tumors and not in normal tissues. (B) The MAGE-B2 pMHC frequencies in representative tumors measured by MS are shown in the table.

FIG. 3. The patient populations in specified cancer indications were estimated based on pMHC target frequency multiplied by new cases (new patient number) per year in U.S. populations. The pMHC target frequency in each cancer indication was calculated by MAGE-B2 and/or MAGE-A4 mRNA expression frequency (TCGA) multiplied by the HLA-A*02:01 carrier frequency in U.S. populations (0.41). MAGE-B2/A4 indicates MAGE-B2 and/or MAGE-A4 positive cancer patients.

FIG. 4. Identification of MAGE-B2 pMHC-specific TCRs from healthy human PBMCs. (A) A schematic illustrates the procedure of identifying MAGE-B2 pMHC-specific TCRs from rare T cell clones isolated from healthy HLA-A*02:01+ donor PBMCs. (B) Flow cytometric identification of MAGE-B2 pMHC-specific T cells by pMHC dextramers (Dex) labelled with two fluorochromes (PE and APC) following multiple rounds of enrichment through stimulation with MAGE-B2 peptide-loaded autologous antigen presenting cells. Representative screen results demonstrate that a positive donor A showed the enriched MAGE-B2 pMHC-specific T cells after multiple ex vivo stimulation, whereas a negative donor B did not have Dex+ T cells. (C) IFNγ ELISPOT analysis of sorted CD8+Dex+ T cells that were stimulated with T2 cells pulsed with a MAGE-B2 peptide or an irrelevant AFP peptide as a negative control.

FIG. 5. MAGE-B2 TCR screen using Jurkat-luciferase activation assay. The activities of individual TCRs were expressed as the average fold change of the luciferase activity (luminescence) in the presence of T2 cells loaded with MAGE-B2 peptide compared to T2 cells with vehicle only. Error bars represent the standard errors.

FIG. 6. Selection of top four MAGE-B2 TCR-Ts by various functional assays. (A) Cytotoxicity summary of MAGE-B2 TCR-Ts (EC90 average of peptide concentration (M) or E:T from 3 donors) in T2/MAGE-B2 peptide cytotoxicity assays including peptide titration and E:T titration studies. (B) Cytotoxicity study using T2/peptide assay with MAGE-B2 peptide concentration titration at E:T=1:1. (C) Cytotoxicity study using T2/peptide assay with E:T titration was carried out at 10⁻⁸M of the MAGE-B2 peptide concentration. (For B and C: TCR1 solid circle, TCR2 solid square, TCR3 solid triangle, TCR4 solid inverted triangle, TCR6 solid diamond, TCR7 star, TCR8 open square, TCR11 open diamond, TCR 12 small solid circle. (D) Cytolytic activity of TCR-Ts against SK-Mel-5 cancer cell line that has MAGE-B2 expression of 27.5 FPKM. TCR1 solid circle, TCR2 solid square, TCR8 star. (E) Representative data from cross-reactivity screen against homology-based similar peptides (T2/peptide cytotoxicity assay). MAGE-B2 solid circle, Peptide 9 solid square, Peptide 25 solid triangle, Peptide 46, open triangle, Peptide 75 solid inverted triangle.

FIG. 7. Schematic diagram of the TCR-T-IL12 lentiviral construct containing TCRα and TCRβ chains with a linker of furin cleavage site-SGSG-T2A under EF1α promoter, and IL12 payload under a composite promoter containing six NFAT (nuclear factor of activated T cells) response elements linked to a minimal IL-2 promoter.

FIG. 8. Potency validation of TCR-T-IL12 using the T2/MAGE-B2 peptide cytotoxicity assay. EC90s of peptide concentration (M) from T2/peptide titration studies using 4 TCR-T-IL12 of 3 HLA-A*02:01 donors are listed in the table. E:T ratio (Dextramer+ T cells:T2) was 1:1. TCR1-IL12 solid circle, TCR2-IL12 solid square, TCR3-IL12 open triangle, TCR4-IL12 solid inverted triangle, IL12 RFP open diamond, Mock T cells solid circle.

FIG. 9. TCR4-IL12 cells from 3 donors showed potent cytotoxicity against both MAGE-B2 peptide- and MAGE-A4 peptide-loaded T2 cells in peptide titration studies. MAGE-B2 open diamond/dashed line, MAGE-A4 solid square.

FIG. 10. Potency summary of four TCR-T-IL12 cells against MAGE-B2+ MAGE-A4− cancer cell lines. All 4 TCR-T-IL12s displayed potent cytotoxicity against cancer cell lines. IL12-RFP T cells (NFAT.IL-12.RFP transduced T cells without transgenic TCR) and mock (untransduced) T cells were used as a negative control. Higher than 50% of max specific killing are highlighted in grey.

FIG. 11. Potency summary of TCR4-IL12 against MAGE-A4+ MAGE-B2− cancer cell lines. Higher than 50% of max specific killing are highlighted as grey.

FIG. 12. Potency summary of four TCR-T-IL12 cells against MAGE-B2+ MAGE-A4+ cancer cell lines. TCR4-IL12 and TCR2-IL12 showed potent cytotoxicity against MAGE-B2+ MAGE-A4+ cancer cell lines. Higher than 50% of max specific killing are highlighted as grey.

FIG. 13. Representative potency for four TCR-T-IL12 cells against MAGE-B2+ and/or MAGE-A4+ cancer cell lines. For potency validation, about 40 cancer cell lines have been tested with 4 TCR-T-IL12 cells generated from 2-3 donors. MAGE-B2 and/or MAGE-A4 mRNA expression levels (FPKM, RNAseq) were listed for each cancer cell line. TCR1-IL12 solid circle, TCR2-IL12 solid square, TCR3-IL12 open triangle, TCR4-IL12 solid inverted triangle, IL12 RFP open diamond.

FIG. 14. Peptide-MHC target-specific cytotoxicity of TCR-T-IL12 was validated by MAGE-B2 KO or B2M KO cancer cell lines. (A) DAN-G derived cancer cell lines (WT, MAGE-B2 KO, and B2M KO) were tested with TCR2-IL12 and TCR4-IL12 for cytotoxicity assays with E:T titration. DAN-G WT solid circle, DAN-G MAGE B2 KI (2E9) solid triangle, DAN-G B2M KO solid inverted triangle. (B) 8505C derived cancer cell lines (WT, MAGE-B2 KO, and B2M KO) were tested with TCR2-IL12 and TCR4-IL12 for cytotoxicity assays with E:T titration. Multiple donors confirmed the same results. MAGE-B2 KO efficiency was validated by sequencing. B2M KO efficiency was verified by flow cytometry. 8505C WT solid circle, 8505C neg gRNAs solid square, 8505C MAGE B2 KO solid triangle, 8505C B2M KO solid inverted triangle.

FIG. 15. IL12 payload increased TCR-T cell potency against low target-expressing cells and enhanced the efficacy of CAR-T cells in vivo. (A) Comparison of TCR-T and TCR-T-IL12 cell potency in vitro. The average of max killing for TCR-T or TCR-T-IL12 cells was derived from specific killing activities of TCR-T cells and TCR-T-IL12 cells generated from 3 different donors. (B) Comparison of CAR-T and CAR-T-IL12 cell efficacy in vivo. The efficacies of huEpCAM CAR-T cells with or without IL12 payload were assessed in B16F10-huEpCAM syngeneic mouse tumor model.

FIG. 16. Summary of cross-reactivity screen with full panel similar peptides. SLC16A10 and KLHDC3 were identified based on X-scan-derived motifs, whereas NRXN1 and MAGE-B1 were identified based on sequence homology to target peptide. MAGE-B1 is another cancer testis antigen with extremely restricted normal tissue expression (only in testis). Based on the cytotoxicity assays with cancer cell lines over-expressing a full-length protein or an endogenous protein, there was no similar peptide identified with off-target concern.

FIG. 17. The SLC16A10 putative cross-reactive peptide was further de-risked by TCDD assays with HLA-A*02:01+ cancer cell lines (NCI-H441 and IGR-1) over-expressing SLA16A10 full length-protein (A) and cancer cell lines (LOUCY and MFE-280) expressing the SLC16A10 endogenous protein (B). MAGE-B2 full length protein-overexpressing (OE) cancer cell lines were used as a positive control target cell line (A). IL12-RFP T cells were used as negative control T cells (B).

FIG. 18. Summary of human normal cell reactivity assessment. No increased IFNγ and granzyme B production by TCR2-IL12 and TCR4-IL12 cells was observed against HLA-A*02:01+ human primary normal cells. Representative data from four normal cell types are shown, including human bronchial epithelial cells (hBEpC), human tracheal epithelial cells (hTEpC), human dermal microvascular endothelial cells (HDMEC), and human keratinocytes (Ker.). Fold changes in IFNγ and granzyme B production compared to the control IL12-RFP T cells are shown in the table. Comparable results were obtained from all nine normal cell types tested and for IL-12p70 and TNFα. B-CPAP cancer cell line (MAGE-B2 65.9 FPKM) was used as a positive control of MAGE-B2+ HLA-A*02:01+ cells. Mock (untransduced) T cells or T cells expressing an IL12-RFP construct (with no transgenic TCR) from the same donor were included as negative control effector cells. Additionally, target cells without T cells (labeled as target only) were used as a negative control for the cytotoxicity assays.

FIG. 19. Summary of alloreactivity assessment. No increases greater than or equal to 4-fold in cytokine or granzyme B responses (compared to IL12-RFP control T cells) against the 34 BLCLs tested were observed for any of the four TCRT-T-IL12 cells. Some low-level responses (greater than or equal to 3-fold, but lower than 4-fold, compared to IL12-RFP control cells) were observed for TCR1-IL12 and TCR2-IL12. Comparable results were obtained for IL-12p70 and TNFα production. All 4 TCR-T-IL12 cells demonstrated robust cytokine and granzyme B responses against positive control U266B1 cells (HLA-A*02:01+ MAGE-B2+ MAGE-A4+) pulsed with MAGE-B2 peptide.

### DETAILED DESCRIPTION

The section headings used herein are for organizational purposes only and are not to be construed as limiting the subject matter described. All references cited within the body of this specification are expressly incorporated by reference in their entirety.

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

Provided herein are T-cell receptor (TCR) alpha and beta chain pairs that bind the MAGE-B2 derived peptide GVYDGEEHSV (SEQ ID NO:1) when presented by an HLA class I molecule, preferably HLA-A*02:01. “TCR alpha and beta chain pair” may also be referred to herein as “TCR,” “a TCR,” or “the TCR.” When expressed recombinantly in a cell, e.g., a T cell, the TCR binds to the MAGE-B2 peptide-HLA complex on a cell, e.g., a cancer cell, and such binding leads to activation of the recombinant cell. Activation of the T cell leads to the death or destruction of the cancer cell. Methods of determining T-cell activation are known in the art and provided with the Examples herein.

In preferred embodiments, the potency or cytolytic activity (cytotoxicity) of a recombinant cell of the present invention is defined by (1) 80-100% lysis of HLA-A*02:01 target cells loaded with peptide at ˜100 copies (˜10⁻⁸ M) per cell in a T cell dependent cellular cytotoxicity (TDCC) assay, T2/peptide loading assay or (2) 80-100% lysis of natural pMHC target-positive cancer cell lines.

In certain embodiments, the TCR further binds the MAGE-A4 derived peptide GVYDGREHTV when presented by an HLA class I molecule, preferably HLA-A*02:01. Such TCRs include TCR3, TCR4, TCR6, TCR7, and TCR11.

Each TCR alpha and beta chain comprises variable and constant domains. Within the variable domain (Vα or Vβ) are three CDRs (complementarity determining regions): CDR1, CDR2, and CDR3. The various alpha and beta chains variable domains are distinguishable by their framework along with their CDR1, CDR2, and part of their CDR3 sequences.

In preferred embodiments, the TCR comprises an alpha chain having a CDR3 set forth in SEQ ID Nos:13-23 and a beta chain having a CDR3 set forth in SEQ ID Nos:24-34. The CDR3 region may be determined by commercially available software (e.g. Cellranger; 10× Genomics). The TCR alpha chain may comprise a sequence at least 95%, at least 96%, at least 97%, at least 98%, at least 99%, or 100% identical to the sequence set forth in any of SEQ ID Nos:35-45. The TCR beta chain may comprise a sequence at least 95%, at least 96%, at least 97%, at least 98%, at least 99%, or 100% identical to the sequence set forth in any of SEQ ID Nos:46-56. Methods of determining the identity between two sequences are well-known in the art, e.g., BLAST or Geneious. In certain embodiments, the C-terminal or N-terminal 1, 2, 3, 4, 5, 6, 7, 8, 9, or 10 residues of any of the sequences set forth is any of SEQ ID Nos:35-45 or any of the sequences set forth in any of SEQ ID Nos:46-56 may be truncated or removed. Exemplary TCRs and the corresponding alpha and beta chain CDR3 and full-length SEQ ID Nos. are provided in Table 1A and Table 1B, SEQ ID NOs: 13-56.

TABLE 1A

| | Alpha | Beta | Alpha mature | Beta mature |
|---|---|---|---|---|
| TCR | CDR3 | CDR3 | full-length | full-length |
| 1 | SEQ ID NO: 13 | SEQ ID NO: 24 | SEQ ID NO: 35 | SEQ ID NO: 46 |
| 2 | SEQ ID NO: 14 | SEQ ID NO: 25 | SEQ ID NO: 36 | SEQ ID NO: 47 |
| 3 | SEQ ID NO: 15 | SEQ ID NO: 26 | SEQ ID NO: 37 | SEQ ID NO: 48 |
| 4 | SEQ ID NO: 16 | SEQ ID NO: 27 | SEQ ID NO: 38 | SEQ ID NO: 49 |
| 5 | SEQ ID NO: 17 | SEQ ID NO: 28 | SEQ ID NO: 39 | SEQ ID NO: 50 |
| 6 | SEQ ID NO: 18 | SEQ ID NO: 29 | SEQ ID NO: 40 | SEQ ID NO: 51 |
| 7 | SEQ ID NO: 19 | SEQ ID NO: 30 | SEQ ID NO: 41 | SEQ ID NO: 52 |
| 8 | SEQ ID NO: 20 | SEQ ID NO: 31 | SEQ ID NO: 42 | SEQ ID NO: 53 |
| 9 | SEQ ID NO: 21 | SEQ ID NO: 32 | SEQ ID NO: 43 | SEQ ID NO: 54 |
| 10 | SEQ ID NO: 22 | SEQ ID NO: 33 | SEQ ID NO: 44 | SEQ ID NO: 55 |
| 11 | SEQ ID NO: 23 | SEQ ID NO: 34 | SEQ ID NO: 45 | SEQ ID NO: 56 |

TABLE 1B

| SEQ | | |
|---|---|---|
| ID | | |
| NO: | Description | Sequence |
| 1 | MAGE-B2- | GVYDGEEHSV |
| | derived | |
| | peptide | |
| 2 | MAGE-A4- | GVYDGREHTV |
| | derived | |
| | peptide | |
| 3 | MAGE-A8- | GLYDGREHSV |
| | derived | |
| | peptide | |
| 4 | KEAP1- | GVIDGHIYAV |
| | derived | |
| | peptide | |
| 5 | MB-derived | GLSDGEWQLV |
| | peptide | |
| 6 | ADF-derived | GVMAGDIYSV |
| | peptide | |
| 7 | DPYSL4- | GLYDGPVHEV |
| | derived | |
| | peptide | |
| 8 | CNPD2- | GVYGGSVHEA |
| | derived | |
| | peptide | |
| 9 | MYOF- | FVYDEPGHAV |
| | derived | |
| | peptide | |
| 10 | COX14- | TVYGGYLCSV |
| | derived | |
| | peptide | |
| 11 | STXBP5- | YTYDEAIHSV |
| | derived | |
| | peptide | |
| 12 | SLK-derived | FIVDGVEVSV |
| | peptide | |
| 13 | TCR1 alpha | CAAMKTSYDKVIF |
| | chain CDR3 | |
| 14 | TCR2 alpha | CAVNIPFSNSGGYQKVTF |
| | chain CDR3 | |
| 15 | TCR3 alpha | CALSVLRMDSSYKLIF |
| | chain CDR3 | |
| 16 | TCR4 alpha | CVVSLGTDKLIF |
| | chain CDR3 | |
| 17 | TCR5 alpha | CAPGGNQFYF |
| | chain CDR3 | |
| 18 | TCR6 alpha | CAFFNAGKSTF |
| | chain CDR3 | |
| 19 | TCR7 alpha | CAVRRLGGYQKVTF |
| | chain CDR3 | |
| 20 | TCR8 alpha | CAMRGPTSYGKLTF |
| | chain CDR3 | |
| 21 | TCR9 alpha | CVVSSDMRF |
| | chain CDR3 | |
| 22 | TCR10 alpha | CAVRDNARLMF |
| | chain CDR3 | |
| 23 | TCR11 alpha | CAEKSITSYDKVIF |
| | chain CDR3 | |
| 24 | TCR1 beta | CASSQGQGGYGYTF |
| | chain CDR3 | |
| 25 | TCR2 beta | CASRHPGQYNQPQHF |
| | chain CDR3 | |
| 26 | TCR3 beta | CASSLQGAGQPQHF |
| | chain CDR3 | |
| 27 | TCR4 beta | CATSAQGNYNEQFF |
| | chain CDR3 | |
| 28 | TCR5 beta | CASSGSNQPQHF |
| | chain CDR3 | |
| 29 | TCR6 beta | CASTVGGGPYGYTF |
| | chain CDR3 | |
| 30 | TCR7 beta | CASSLVTGSSYNEQFF |
| | chain CDR3 | |
| 31 | TCR8 beta | CATSPTTDNQPQHF |
| | chain CDR3 | |
| 32 | TCR9 beta | CASSYGGDEQYF |
| | chain CDR3 | |
| 33 | TCR10 beta | CSVGPSGHTGYTF |
| | chain CDR3 | |
| 34 | TCR11 beta | CASTRRGTYGYTF |
| | chain CDR3 | |
| 35 | TCR1 alpha | KQEVTQIPAALSVPEGENLVLNCSFTDSAIYNLQWFRQDPG |
| | chain mature | KGLTSLLLIQSSQREQTSGRLNASLDKSSGRSTLYIAASQPGD |
| | | SATYLCAAMKTSYDKVIFGPGTSLSVIPNIQNPDPAVYQLRD |
| | | SKSSDKSVCLFTDFDSQTNVSQSKDSDVYITDKTVLDMRSM |
| | | DFKSNSAVAWSNKSDFACANAFNNSIIPEDTFFPSPESSCDV |
| | | KLVEKSFETDTNLNFQNLSVIGFRILLLKVAGFNLLMTLRLW |
| | | SS |
| 36 | TCR2 alpha | QKEVEQNSGPLSVPEGAIASLNCTYSDRGSQSFFWYRQYSG |
| | chain mature | KSPELIMSIYSNGDKEDGRFTAQLNKASQYVSLLIRDSQPSD |
| | | SATYLCAVNIPFSNSGGYQKVTFGTGTKLQVIPNIQNPDPAV |
| | | YQLRDSKSSDKSVCLFTDFDSQTNVSQSKDSDVYITDKTVL |
| | | DMRSMDFKSNSAVAWSNKSDFACANAFNNSIIPEDTFFPSPE |
| | | SSCDVKLVEKSFETDTNLNFQNLSVIGFRILLLKVAGFNLLM |
| | | TLRLWSS |
| 37 | TCR3 alpha | AQKVTQAQTEISVVEKEDVTLDCVYETRDTTYYLFWYKQP |
| | chain mature | PSGELVFLIRRNSFDEQNEISGRYSWNFQKSTSSFNFTITASQ |
| | | VVDSAVYFCALSVLRMDSSYKLIFGSGTRLLVRPDIQNPDPA |
| | | VYQLRDSKSSDKSVCLFTDFDSQTNVSQSKDSDVYITDKTV |
| | | LDMRSMDFKSNSAVAWSNKSDFACANAFNNSIIPEDTFFPSP |
| | | ESSCDVKLVEKSFETDTNLNFQNLSVIGFRILLLKVAGENLL |
| | | MTLRLWSS |
| 38 | TCR4 alpha | KNQVEQSPQSLIILEGKNCTLQCNYTVSPFSNLRWYKQDTG |
| | chain mature | RGPVSLTIMTFSENTKSNGRYTATLDADTKQSSLHITASQLS |
| | | DSASYICVVSLGTDKLIFGTGTRLQVFPNIQNPDPAVYQLRD |
| | | SKSSDKSVCLFTDFDSQTNVSQSKDSDVYITDKTVLDMRSM |
| | | DFKSNSAVAWSNKSDFACANAFNNSIIPEDTFFPSPESSCDV |
| | | KLVEKSFETDTNLNFQNLSVIGFRILLLKVAGFNLLMTLRLW |
| | | SS |
| 39 | TCR5 alpha | KNEVEQSPQNLTAQEGEFITINCSYSVGISALHWLQQHPGGG |
| | chain mature | IVSLFMLSSGKKKHGRLIATINIQEKHSSLHITASHPRDSAVY |
| | | ICAPGGNQFYFGTGTSLTVIPNIQNPDPAVYQLRDSKSSDKS |
| | | VCLFTDFDSQTNVSQSKDSDVYITDKTVLDMRSMDFKSNSA |
| | | VAWSNKSDFACANAFNNSIIPEDTFFPSPESSCDVKLVEKSF |
| | | ETDTNLNFQNLSVIGFRILLLKVAGFNLLMTLRLWSS |
| 40 | TCR6 alpha | AQTVTQSQPEMSVQEAETVTLSCTYDTSENNYYLFWYKQP |
| | chain mature | PSRQMILVIRQEAYKQQNATENRFSVNFQKAAKSFSLKISDS |
| | | QLGDTAMYFCAFFNAGKSTFGDGTTLTVKPNIQNPDPAVY |
| | | QLRDSKSSDKSVCLFTDFDSQTNVSQSKDSDVYITDKTVLD |
| | | MRSMDFKSNSAVAWSNKSDFACANAFNNSIIPEDTFFPSPES |
| | | SCDVKLVEKSFETDTNLNFQNLSVIGFRILLLKVAGFNLLMT |
| | | LRLWSS |
| 41 | TCR7 alpha | GQNIDQPTEMTATEGAIVQINCTYQTSGFNGLFWYQQHAGE |
| | chain mature | APTFLSYNVLDGLEEKGRFSSFLSRSKGYSYLLLKELQMKD |
| | | SASYLCAVRRLGGYQKVTFGTGTKLQVIPNIQNPDPAVYQL |
| | | RDSKSSDKSVCLFTDFDSQTNVSQSKDSDVYITDKTVLDMR |
| | | SMDFKSNSAVAWSNKSDFACANAFNNSIIPEDTFFPSPESSC |
| | | DVKLVEKSFETDTNLNFQNLSVIGFRILLLKVAGFNLLMTLR |
| | | LWSS |
| 42 | TCR8 alpha | AQKITQTQPGMFVQEKEAVTLDCTYDTSDQSYGLFWYKQP |
| | chain mature | SSGEMIFLIYQGSYDEQNATEGRYSLNFQKARKSANLVISAS |
| | | QLGDSAMYFCAMRGPTSYGKLTFGQGTILTVHPNIQNPDPA |
| | | VYQLRDSKSSDKSVCLFTDFDSQTNVSQSKDSDVYITDKTV |
| | | LDMRSMDFKSNSAVAWSNKSDFACANAFNNSIIPEDTFFPSP |
| | | ESSCDVKLVEKSFETDTNLNFQNLSVIGFRILLLKVAGFNLL |
| | | MTLRLWSS |
| 43 | TCR9 alpha | KNQVEQSPQSLIILEGKNCTLQCNYTVSPFSNLRWYKQDTG |
| | chain mature | RGPVSLTIMTFSENTKSNGRYTATLDADTKQSSLHITASQLS |
| | | DSASYICVVSSDMRFGAGTRLTVKPNIQNPDPAVYQLRDSK |
| | | SSDKSVCLFTDFDSQTNVSQSKDSDVYITDKTVLDMRSMDF |
| | | KSNSAVAWSNKSDFACANAFNNSIIPEDTFFPSPESSCDVKL |
| | | VEKSFETDTNLNFQNLSVIGFRILLLKVAGFNLLMTLRLWSS |
| 44 | TCR10 alpha | AQSVAQPEDQVNVAEGNPLTVKCTYSVSGNPYLFWYVQYP |
| | chain mature | NRGLQFLLKYITGDNLVKGSYGFEAEFNKSQTSFHLKKPSA |
| | | LVSDSALYFCAVRDNARLMFGDGTQLVVKPNIQNPDPAVY |
| | | QLRDSKSSDKSVCLFTDFDSQTNVSQSKDSDVYITDKTVLD |
| | | MRSMDFKSNSAVAWSNKSDFACANAFNNSIIPEDTFFPSPES |
| | | SCDVKLVEKSFETDTNLNFQNLSVIGFRILLLKVAGFNLLMT |
| | | LRLWSS |
| 45 | TCR11 alpha | GEDVEQSLFLSVREGDSSVINCTYTDSSSTYLYWYKQEPGA |
| | chain mature | GLQLLTYIFSNMDMKQDQRLTVLLNKKDKHLSLRIADTQT |
| | | GDSAIYFCAEKSITSYDKVIFGPGTSLSVIPNIQNPDPAVYQL |
| | | RDSKSSDKSVCLFTDFDSQTNVSQSKDSDVYITDKTVLDMR |
| | | SMDFKSNSAVAWSNKSDFACANAFNNSIIPEDTFFPSPESSC |
| | | DVKLVEKSFETDTNLNFQNLSVIGFRILLLKVAGFNLLMTLR |
| | | LWSS |
| 46 | TCR1 beta | GEEVAQTPKHLVRGEGQKAKLYCAPIKGHSYVFWYQQVLK |
| | chain mature | NEFKFLISFQNENVFDETGMPKERFSAKCLPNSPCSLEIQATK |
| | | LEDSAVYFCASSQGQGGYGYTFGSGTRLTVVEDLNKVFPPE |
| | | VAVFEPSEAEISHTQKATLVCLATGFFPDHVELSWWVNGKE |
| | | VHSGVSTDPQPLKEQPALNDSRYCLSSRLRVSATFWQNPRN |
| | | HFRCQVQFYGLSENDEWTQDRAKPVTQIVSAEAWGRADCG |
| | | FTSVSYQQGVLSATILYEILLGKATLYAVLVSALVLMAMVK |
| | | RKDF |
| 47 | TCR2 beta | NAGVTQTPKFRVLKTGQSMTLLCAQDMNHEYMYWYRQDP |
| | chain mature | GMGLRLIHYSVGEGTTAKGEVPDGYNVSRLKKQNFLLGLE |
| | | SAAPSQTSVYFCASRHPGQYNQPQHFGDGTRLSILEDLNKV |
| | | FPPEVAVFEPSEAEISHTQKATLVCLATGFFPDHVELSWWV |
| | | NGKEVHSGVSTDPQPLKEQPALNDSRYCLSSRLRVSATFWQ |
| | | NPRNHFRCQVQFYGLSENDEWTQDRAKPVTQIVSAEAWGR |
| | | ADCGFTSVSYQQGVLSATILYEILLGKATLYAVLVSALVLM |
| | | AMVKRKDF |
| 48 | TCR3 beta | NAGVTQTPKFRVLKTGQSMTLLCAQDMNHEYMYWYRQDP |
| | chain mature | GMGLRLIHYSVGEGTTAKGEVPDGYNVSRLKKQNFLLGLE |
| | | SAAPSQTSVYFCASSLQGAGQPQHFGDGTRLSILEDLNKVFP |
| | | PEVAVFEPSEAEISHTQKATLVCLATGFFPDHVELSWWVNG |
| | | KEVHSGVSTDPQPLKEQPALNDSRYCLSSRLRVSATFWQNP |
| | | RNHFRCQVQFYGLSENDEWTQDRAKPVTQIVSAEAWGRAD |
| | | CGFTSVSYQQGVLSATILYEILLGKATLYAVLVSALVLMAM |
| | | VKRKDF |
| 49 | TCR4 beta | DADVTQTPRNRITKTGKRIMLECSQTKGHDRMYWYRQDPG |
| | chain mature | LGLRLIYYSFDVKDINKGEISDGYSVSRQAQAKFSLSLESAIP |
| | | NQTALYFCATSAQGNYNEQFFGPGTRLTVLEDLKNVFPPEV |
| | | AVFEPSEAEISHTQKATLVCLATGFYPDHVELSWWVNGKE |
| | | VHSGVSTDPQPLKEQPALNDSRYCLSSRLRVSATFWQNPRN |
| | | HFRCQVQFYGLSENDEWTQDRAKPVTQIVSAEAWGRADCG |
| | | FTSESYQQGVLSATILYEILLGKATLYAVLVSALVLMAMVK |
| | | RKDSRG |
| 50 | TCR5 beta | DAGITQSPRYKITETGRQVTLMCHQTWSHSYMFWYRQDLG |
| | chain mature | HGLRLIYYSAAADITDKGEVPDGYVVSRSKTENFPLTLESAT |
| | | RSQTSVYFCASSGSNQPQHFGDGTRLSILEDLNKVFPPEVAV |
| | | FEPSEAEISHTQKATLVCLATGFFPDHVELSWWVNGKEVHS |
| | | GVSTDPQPLKEQPALNDSRYCLSSRLRVSATFWQNPRNHFR |
| | | CQVQFYGLSENDEWTQDRAKPVTQIVSAEAWGRADCGFTS |
| | | VSYQQGVLSATILYEILLGKATLYAVLVSALVLMAMVKRK |
| | | DF |
| 51 | TCR6 beta | EAGVAQSPRYKIIEKRQSVAFWCNPISGHATLYWYQQILGQ |
| | chain mature | GPKLLIQFQNNGVVDDSQLPKDRFSAERLKGVDSTLKIQPA |
| | | KLEDSAVYLCASTVGGGPYGYTFGSGTRLTVVEDLNKVFPP |
| | | EVAVFEPSEAEISHTQKATLVCLATGFFPDHVELSWWVNGK |
| | | EVHSGVSTDPQPLKEQPALNDSRYCLSSRLRVSATFWQNPR |
| | | NHFRCQVQFYGLSENDEWTQDRAKPVTQIVSAEAWGRADC |
| | | GFTSVSYQQGVLSATILYEILLGKATLYAVLVSALVLMAMV |
| | | KRKDF |
| 52 | TCR7 beta | GAGVSQSPSNKVTEKGKDVELRCDPISGHTALYWYRQRLG |
| | chain mature | QGLEFLIYFQGNSAPDKSGLPSDRFSAERTGESVSTLTIQRTQ |
| | | QEDSAVYLCASSLVTGSSYNEQFFGPGTRLTVLEDLKNVFPP |
| | | EVAVFEPSEAEISHTQKATLVCLATGFYPDHVELSWWVNG |
| | | KEVHSGVSTDPQPLKEQPALNDSRYCLSSRLRVSATFWQNP |
| | | RNHFRCQVQFYGLSENDEWTQDRAKPVTQIVSAEAWGRAD |
| | | CGFTSESYQQGVLSATILYEILLGKATLYAVLVSALVLMAM |
| | | VKRKDSRG |
| 53 | TCR8 beta | DADVTQTPRNRITKTGKRIMLECSQTKGHDRMYWYRQDPG |
| | chain mature | LGLRLIYYSFDVKDINKGEISDGYSVSRQAQAKFSLSLESAIP |
| | | NQTALYFCATSPTTDNQPQHFGDGTRLSILEDLNKVFPPEVA |
| | | VFEPSEAEISHTQKATLVCLATGFFPDHVELSWWVNGKEVH |
| | | SGVSTDPQPLKEQPALNDSRYCLSSRLRVSATFWQNPRNHF |
| | | RCQVQFYGLSENDEWTQDRAKPVTQIVSAEAWGRADCGFT |
| | | SVSYQQGVLSATILYEILLGKATLYAVLVSALVLMAMVKR |
| | | KDF |
| 54 | TCR9 beta | NAGVTQTPKFRILKIGQSMTLQCAQDMNHNYMYWYRQDP |
| | chain mature | GMGLKLIYYSVGAGITDKGEVPNGYNVSRSTTEDFPLRLEL |
| | | AAPSQTSVYFCASSYGGDEQYFGPGTRLTVTEDLKNVFPPE |
| | | VAVFEPSEAEISHTQKATLVCLATGFYPDHVELSWWVNGK |
| | | EVHSGVSTDPQPLKEQPALNDSRYCLSSRLRVSATFWQNPR |
| | | NHFRCQVQFYGLSENDEWTQDRAKPVTQIVSAEAWGRADC |
| | | GFTSESYQQGVLSATILYEILLGKATLYAVLVSALVLMAMV |
| | | KRKDSRG |
| 55 | TCR10 beta | SAVISQKPSRDICQRGTSLTIQCQVDSQVTMMFWYRQQPGQ |
| | chain mature | SLTLIATANQGSEATYESGFVIDKFPISRPNLTFSTLTVSNMS |
| | | PEDSSIYLCSVGPSGHTGYTFGSGTRLTVVEDLNKVFPPEVA |
| | | VFEPSEAEISHTQKATLVCLATGFFPDHVELSWWVNGKEVH |
| | | SGVSTDPQPLKEQPALNDSRYCLSSRLRVSATFWQNPRNHF |
| | | RCQVQFYGLSENDEWTQDRAKPVTQIVSAEAWGRADCGFT |
| | | SVSYQQGVLSATILYEILLGKATLYAVLVSALVLMAMVKR |
| | | KDF |
| 56 | TCR11 beta | DVKVTQSSRYLVKRTGEKVFLECVQDMDHENMFWYRQDP |
| | chain mature | GLGLRLIYFSYDVKMKEKGDIPEGYSVSREKKERFSLILESA |
| | | STNQTSMYLCASTRRGTYGYTFGSGTRLTVVEDLNKVFPPE |
| | | VAVFEPSEAEISHTQKATLVCLATGFFPDHVELSWWVNGKE |
| | | VHSGVSTDPQPLKEQPALNDSRYCLSSRLRVSATFWQNPRN |
| | | HFRCQVQFYGLSENDEWTQDRAKPVTQIVSAEAWGRADCG |
| | | FTSVSYQQGVLSATILYEILLGKATLYAVLVSALVLMAMVK |
| | | RKDF |
| 57 | TCR1 alpha | METLLGLLILWLQLQWVSSKQEVTQIPAALSVPEGENLVLN |
| | chain with | CSFTDSAIYNLQWFRQDPGKGLTSLLLIQSSQREQTSGRLNA |
| | signal peptide | SLDKSSGRSTLYIAASQPGDSATYLCAAMKTSYDKVIFGPGT |
| | | SLSVIPNIQNPDPAVYQLRDSKSSDKSVCLFTDFDSQTNVSQ |
| | | SKDSDVYITDKTVLDMRSMDFKSNSAVAWSNKSDFACANA |
| | | FNNSIIPEDTFFPSPESSCDVKLVEKSFETDTNLNFQNLSVIGF |
| | | RILLLKVAGFNLLMTLRLWSS |
| 58 | TCR2 alpha | MKSLRVLLVILWLQLSWVWSQQKEVEQNSGPLSVPEGAIAS |
| | chain with | LNCTYSDRGSQSFFWYRQYSGKSPELIMSIYSNGDKEDGRF |
| | signal peptide | TAQLNKASQYVSLLIRDSQPSDSATYLCAVNIPFSNSGGYQK |
| | | VTFGTGTKLQVIPNIQNPDPAVYQLRDSKSSDKSVCLFTDFD |
| | | SQTNVSQSKDSDVYITDKTVLDMRSMDFKSNSAVAWSNKS |
| | | DFACANAFNNSIIPEDTFFPSPESSCDVKLVEKSFETDTNLNF |
| | | QNLSVIGFRILLLKVAGFNLLMTLRLWSS |
| 59 | TCR3 alpha | MLTASLLRAVIASICVVSSMAQKVTQAQTEISVVEKEDVTL |
| | chain with | DCVYETRDTTYYLFWYKQPPSGELVFLIRRNSFDEQNEISGR |
| | signal peptide | YSWNFQKSTSSFNFTITASQVVDSAVYFCALSVLRMDSSYK |
| | | LIFGSGTRLLVRPDIQNPDPAVYQLRDSKSSDKSVCLFTDFD |
| | | SQTNVSQSKDSDVYITDKTVLDMRSMDFKSNSAVAWSNKS |
| | | DFACANAFNNSIIPEDTFFPSPESSCDVKLVEKSFETDTNLNF |
| | | QNLSVIGFRILLLKVAGFNLLMTLRLWSS |
| 60 | TCR4 alpha | MKKHLTTFLVILWLYFYRGNGKNQVEQSPQSLIILEGKNCT |
| | chain with | LQCNYTVSPFSNLRWYKQDTGRGPVSLTIMTFSENTKSNGR |
| | signal peptide | YTATLDADTKQSSLHITASQLSDSASYICVVSLGTDKLIFGT |
| | r | GTRLQVFPNIQNPDPAVYQLRDSKSSDKSVCLFTDFDSQTN |
| | | VSQSKDSDVYITDKTVLDMRSMDFKSNSAVAWSNKSDFAC |
| | | ANAFNNSIIPEDTFFPSPESSCDVKLVEKSFETDTNLNFQNLS |
| | | VIGFRILLLKVAGFNLLMTLRLWSS |
| 61 | TCR5 alpha | MVKIRQFLLAILWLQLSCVSAAKNEVEQSPQNLTAQEGEFIT |
| | chain with | INCSYSVGISALHWLQQHPGGGIVSLFMLSSGKKKHGRLIAT |
| | signal peptide | INIQEKHSSLHITASHPRDSAVYICAPGGNQFYFGTGTSLTVI |
| | | PNIQNPDPAVYQLRDSKSSDKSVCLFTDFDSQTNVSQSKDS |
| | | DVYITDKTVLDMRSMDFKSNSAVAWSNKSDFACANAFNNS |
| | | IIPEDTFFPSPESSCDVKLVEKSFETDTNLNFQNLSVIGFRILL |
| | | LKVAGFNLLMTLRLWSS |
| 62 | TCR6 alpha | MTRVSLLWAVVVSTCLESGMAQTVTQSQPEMSVQEAETVT |
| | chain with | LSCTYDTSENNYYLFWYKQPPSRQMILVIRQEAYKQQNATE |
| | signal peptide | NRFSVNFQKAAKSFSLKISDSQLGDTAMYFCAFFNAGKSTF |
| | | GDGTTLTVKPNIQNPDPAVYQLRDSKSSDKSVCLFTDFDSQ |
| | | TNVSQSKDSDVYITDKTVLDMRSMDFKSNSAVAWSNKSDF |
| | | ACANAFNNSIIPEDTFFPSPESSCDVKLVEKSFETDTNLNFQN |
| | | LSVIGFRILLLKVAGFNLLMTLRLWSS |
| 63 | TCR7 alpha | MWGVFLLYVSMKMGGTTGQNIDQPTEMTATEGAIVQINCT |
| | chain with | YQTSGFNGLFWYQQHAGEAPTFLSYNVLDGLEEKGRFSSFL |
| | signal peptide | SRSKGYSYLLLKELQMKDSASYLCAVRRLGGYQKVTFGTG |
| | | TKLQVIPNIQNPDPAVYQLRDSKSSDKSVCLFTDFDSQTNVS |
| | | QSKDSDVYITDKTVLDMRSMDFKSNSAVAWSNKSDFACAN |
| | | AFNNSIIPEDTFFPSPESSCDVKLVEKSFETDTNLNFQNLSVIG |
| | | FRILLLKVAGFNLLMTLRLWSS |
| 64 | TCR8 alpha | MSLSSLLKVVTASLWLGPGIAQKITQTQPGMFVQEKEAVTL |
| | chain with | DCTYDTSDQSYGLFWYKQPSSGEMIFLIYQGSYDEQNATEG |
| | signal peptide | RYSLNFQKARKSANLVISASQLGDSAMYFCAMRGPTSYGK |
| | | LTFGQGTILTVHPNIQNPDPAVYQLRDSKSSDKSVCLFTDFD |
| | | SQTNVSQSKDSDVYITDKTVLDMRSMDFKSNSAVAWSNKS |
| | | DFACANAFNNSIIPEDTFFPSPESSCDVKLVEKSFETDTNLNF |
| | | QNLSVIGFRILLLKVAGFNLLMTLRLWSS |
| 65 | TCR9 alpha | MKKHLTTFLVILWLYFYRGNGKNQVEQSPQSLIILEGKNCT |
| | chain with | LQCNYTVSPFSNLRWYKQDTGRGPVSLTIMTFSENTKSNGR |
| | signal peptide | YTATLDADTKQSSLHITASQLSDSASYICVVSSDMRFGAGTR |
| | | LTVKPNIQNPDPAVYQLRDSKSSDKSVCLFTDFDSQTNVSQS |
| | | KDSDVYITDKTVLDMRSMDFKSNSAVAWSNKSDFACANAF |
| | | NNSIIPEDTFFPSPESSCDVKLVEKSFETDTNLNFQNLSVIGFR |
| | | ILLLKVAGFNLLMTLRLWSS |
| 66 | TCR10 alpha | MASAPISMLAMLFTLSGLRAQSVAQPEDQVNVAEGNPLTV |
| | chain with | KCTYSVSGNPYLFWYVQYPNRGLQFLLKYITGDNLVKGSY |
| | signal peptide | GFEAEFNKSQTSFHLKKPSALVSDSALYFCAVRDNARLMFG |
| | | DGTQLVVKPNIQNPDPAVYQLRDSKSSDKSVCLFTDFDSQT |
| | | NVSQSKDSDVYITDKTVLDMRSMDFKSNSAVAWSNKSDFA |
| | | CANAFNNSIIPEDTFFPSPESSCDVKLVEKSFETDTNLNFQNL |
| | | SVIGFRILLLKVAGFNLLMTLRLWSS |
| 67 | TCR11 alpha | MKTFAGFSFLFLWLQLDCMSRGEDVEQSLFLSVREGDSSVI |
| | chain with | NCTYTDSSSTYLYWYKQEPGAGLQLLTYIFSNMDMKQDQR |
| | signal peptide | LTVLLNKKDKHLSLRIADTQTGDSAIYFCAEKSITSYDKVIF |
| | | GPGTSLSVIPNIQNPDPAVYQLRDSKSSDKSVCLFTDFDSQT |
| | | NVSQSKDSDVYITDKTVLDMRSMDFKSNSAVAWSNKSDFA |
| | | CANAFNNSIIPEDTFFPSPESSCDVKLVEKSFETDTNLNFQNL |
| | | SVIGFRILLLKVAGFNLLMTLRLWSS |
| 68 | TCR1 beta | MSPIFTCITILCLLAAGSPGEEVAQTPKHLVRGEGQKAKLYC |
| | chain with | APIKGHSYVFWYQQVLKNEFKFLISFQNENVFDETGMPKER |
| | signal peptide | FSAKCLPNSPCSLEIQATKLEDSAVYFCASSQGQGGYGYTFG |
| | | SGTRLTVVEDLNKVFPPEVAVFEPSEAEISHTQKATLVCLAT |
| | | GFFPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSVSYQQGVLSATILYEILLGK |
| | | ATLYAVLVSALVLMAMVKRKDF |
| 69 | TCR2 beta | MSLGLLCCGAFSLLWAGPVNAGVTQTPKFRVLKTGQSMTL |
| | chain with | LCAQDMNHEYMYWYRQDPGMGLRLIHYSVGEGTTAKGEV |
| | signal peptide | PDGYNVSRLKKQNFLLGLESAAPSQTSVYFCASRHPGQYNQ |
| | | PQHFGDGTRLSILEDLNKVFPPEVAVFEPSEAEISHTQKATL |
| | | VCLATGFFPDHVELSWWVNGKEVHSGVSTDPQPLKEQPAL |
| | | NDSRYCLSSRLRVSATFWQNPRNHFRCQVQFYGLSENDEW |
| | | TQDRAKPVTQIVSAEAWGRADCGFTSVSYQQGVLSATILYE |
| | | ILLGKATLYAVLVSALVLMAMVKRKDF |
| 70 | TCR3 beta | MSLGLLCCAAFSLLWAGPVNAGVTQTPKFRVLKTGQSMTL |
| | chain with | LCAQDMNHEYMYWYRQDPGMGLRLIHYSVGEGTTAKGEV |
| | signal peptide | PDGYNVSRLKKQNFLLGLESAAPSQTSVYFCASSLQGAGQP |
| | | QHFGDGTRLSILEDLNKVFPPEVAVFEPSEAEISHTQKATLV |
| | | CLATGFFPDHVELSWWVNGKEVHSGVSTDPQPLKEQPALN |
| | | DSRYCLSSRLRVSATFWQNPRNHFRCQVQFYGLSENDEWT |
| | | QDRAKPVTQIVSAEAWGRADCGFTSVSYQQGVLSATILYEI |
| | | LLGKATLYAVLVSALVLMAMVKRKDF |
| 71 | TCR4 beta | MASLLFFCGAFYLLGTGSMDADVTQTPRNRITKTGKRIMLE |
| | chain with | CSQTKGHDRMYWYRQDPGLGLRLIYYSFDVKDINKGEISD |
| | signal peptide | GYSVSRQAQAKFSLSLESAIPNQTALYFCATSAQGNYNEQF |
| | | FGPGTRLTVLEDLKNVFPPEVAVFEPSEAEISHTQKATLVCL |
| | | ATGFYPDHVELSWWVNGKEVHSGVSTDPQPLKEQPALNDS |
| | | RYCLSSRLRVSATFWQNPRNHFRCQVQFYGLSENDEWTQD |
| | | RAKPVTQIVSAEAWGRADCGFTSESYQQGVLSATILYEILLG |
| | | KATLYAVLVSALVLMAMVKRKDSRG |
| 72 | TCR5 beta | MGTRLFFYVALCLLWAGHRDAGITQSPRYKITETGRQVTL |
| | chain with | MCHQTWSHSYMFWYRQDLGHGLRLIYYSAAADITDKGEV |
| | signal peptide | PDGYVVSRSKTENFPLTLESATRSQTSVYFCASSGSNQPQHF |
| | | GDGTRLSILEDLNKVFPPEVAVFEPSEAEISHTQKATLVCLA |
| | | TGFFPDHVELSWWVNGKEVHSGVSTDPQPLKEQPALNDSR |
| | | YCLSSRLRVSATFWQNPRNHFRCQVQFYGLSENDEWTQDR |
| | | AKPVTQIVSAEAWGRADCGFTSVSYQQGVLSATILYEILLG |
| | | KATLYAVLVSALVLMAMVKRKDF |
| 73 | TCR6 beta | MGTRLLCWAALCLLGAELTEAGVAQSPRYKIIEKRQSVAF |
| | chain with | WCNPISGHATLYWYQQILGQGPKLLIQFQNNGVVDDSQLPK |
| | signal peptide | DRFSAERLKGVDSTLKIQPAKLEDSAVYLCASTVGGGPYGY |
| | | TFGSGTRLTVVEDLNKVFPPEVAVFEPSEAEISHTQKATLVC |
| | | LATGFFPDHVELSWWVNGKEVHSGVSTDPQPLKEQPALND |
| | | SRYCLSSRLRVSATFWQNPRNHFRCQVQFYGLSENDEWTQ |
| | | DRAKPVTQIVSAEAWGRADCGFTSVSYQQGVLSATILYEIL |
| | | LGKATLYAVLVSALVLMAMVKRKDF |
| 74 | TCR7 beta | MGTRLLFWVAFCLLGAYHTGAGVSQSPSNKVTEKGKDVEL |
| | chain with | RCDPISGHTALYWYRQRLGQGLEFLIYFQGNSAPDKSGLPS |
| | signal peptide | DRFSAERTGESVSTLTIQRTQQEDSAVYLCASSLVTGSSYNE |
| | | QFFGPGTRLTVLEDLKNVFPPEVAVFEPSEAEISHTQKATLV |
| | | CLATGFYPDHVELSWWVNGKEVHSGVSTDPQPLKEQPALN |
| | | DSRYCLSSRLRVSATFWQNPRNHFRCQVQFYGLSENDEWT |
| | | QDRAKPVTQIVSAEAWGRADCGFTSESYQQGVLSATILYEI |
| | | LLGKATLYAVLVSALVLMAMVKRKDSRG |
| 75 | TCR8 beta | MASLLFFCGAFYLLGTGSMDADVTQTPRNRITKTGKRIMLE |
| | chain with | CSQTKGHDRMYWYRQDPGLGLRLIYYSFDVKDINKGEISD |
| | signal peptide | GYSVSRQAQAKFSLSLESAIPNQTALYFCATSPTTDNQPQHF |
| | | GDGTRLSILEDLNKVFPPEVAVFEPSEAEISHTQKATLVCLA |
| | | TGFFPDHVELSWWVNGKEVHSGVSTDPQPLKEQPALNDSR |
| | | YCLSSRLRVSATFWQNPRNHFRCQVQFYGLSENDEWTQDR |
| | | AKPVTQIVSAEAWGRADCGFTSVSYQQGVLSATILYEILLG |
| | | KATLYAVLVSALVLMAMVKRKDF |
| 76 | TCR9 beta | MSISLLCCAAFPLLWAGPVNAGVTQTPKFRILKIGQSMTLQC |
| | chain with | AQDMNHNYMYWYRQDPGMGLKLIYYSVGAGITDKGEVPN |
| | signal peptide | GYNVSRSTTEDFPLRLELAAPSQTSVYFCASSYGGDEQYFGP |
| | | GTRLTVTEDLKNVFPPEVAVFEPSEAEISHTQKATLVCLATG |
| | | FYPDHVELSWWVNGKEVHSGVSTDPQPLKEQPALNDSRYC |
| | | LSSRLRVSATFWQNPRNHFRCQVQFYGLSENDEWTQDRAK |
| | | PVTQIVSAEAWGRADCGFTSESYQQGVLSATILYEILLGKAT |
| | | LYAVLVSALVLMAMVKRKDSRG |
| 77 | TCR10 beta | MLSLLLLLLGLGSVFSAVISQKPSRDICQRGTSLTIQCQVDSQ |
| | chain with | VTMMFWYRQQPGQSLTLIATANQGSEATYESGFVIDKFPIS |
| | signal peptide | RPNLTFSTLTVSNMSPEDSSIYLCSVGPSGHTGYTFGSGTRLT |
| | | VVEDLNKVFPPEVAVFEPSEAEISHTQKATLVCLATGFFPDH |
| | | VELSWWVNGKEVHSGVSTDPQPLKEQPALNDSRYCLSSRL |
| | | RVSATFWQNPRNHFRCQVQFYGLSENDEWTQDRAKPVTQI |
| | | VSAEAWGRADCGFTSVSYQQGVLSATILYEILLGKATLYAV |
| | | LVSALVLMAMVKRKDF |
| 78 | TCR11 beta | MGIRLLCRVAFCFLAVGLVDVKVTQSSRYLVKRTGEKVFLE |
| | chain with | CVQDMDHENMFWYRQDPGLGLRLIYFSYDVKMKEKGDIPE |
| | signal peptide | GYSVSREKKERFSLILESASTNQTSMYLCASTRRGTYGYTFG |
| | | SGTRLTVVEDLNKVFPPEVAVFEPSEAEISHTQKATLVCLAT |
| | | GFFPDHVELSWWVNGKEVHSGVSTDPQPLKEQPALNDSRY |
| | | CLSSRLRVSATFWQNPRNHFRCQVQFYGLSENDEWTQDRA |
| | | KPVTQIVSAEAWGRADCGFTSVSYQQGVLSATILYEILLGK |
| | | ATLYAVLVSALVLMAMVKRKDF |

In certain embodiments, the variable domain of a TCR alpha or beta chain may be fused to a non-TCR polypeptide. The exemplary alpha and beta chain variable domains may be used to create a soluble TCR capable of binding the MAGE-B2 (and in some instances MAGE-A4) derived peptide in the context of an HLA molecule. The soluble TCRs may be in single chain format wherein the alpha and beta variable domains are connected by a linker. A disulfide bond may be introduced between the alpha and beta chains to increase stability. The soluble TCRs may be fused or connected to a therapeutic or imaging agent.

Exemplary TCRs and the corresponding alpha and beta variable regions are provided in Table 2.

TABLE 2

| TCR | Alpha variable domain | Beta variable domain |
|---|---|---|
| 1 | Amino acids 1-113 SEQ ID NO: 35 | Amino acids 1-114 SEQ ID NO: 46 |
| 2 | Amino acids 1-117 SEQ ID NO: 36 | Amino acids 1-114 SEQ ID NO: 47 |
| 3 | Amino acids 1-118 SEQ ID NO: 37 | Amino acids 1-113 SEQ ID NO: 48 |
| 4 | Amino acids 1-112 SEQ ID NO: 38 | Amino acids 1-113 SEQ ID NO: 49 |
| 5 | Amino acids 1-107 SEQ ID NO: 39 | Amino acids 1-111 SEQ ID NO: 50 |
| 6 | Amino acids 1-113 SEQ ID NO: 40 | Amino acids 1-114 SEQ ID NO: 51 |
| 7 | Amino acids 1-112 SEQ ID NO: 41 | Amino acids 1-116 SEQ ID NO: 52 |
| 8 | Amino acids 1-116 SEQ ID NO: 42 | Amino acids 1-113 SEQ ID NO: 53 |
| 9 | Amino acids 1-109 SEQ ID NO: 43 | Amino acids 1-111 SEQ ID NO: 54 |
| 10 | Amino acids 1-112 SEQ ID NO: 44 | Amino acids 1-114 SEQ ID NO: 55 |
| 11 | Amino acids 1-113 SEQ ID NO: 45 | Amino acids 1-112 SEQ ID NO: 56 |

The TCR alpha or beta variable domain may comprise a sequence at least 95%, at least 96%, at least 97%, at least 98%, at least 99%, or 100% identical to any of the sequences specified in Table 2. The TCR beta chain may comprise a sequence at least 80%, at least 85%, at least 90%, at least 95%, at least 96%, at least 97%, at least 98%, at least 99%, or 100% identical to the sequence set forth is any of SEQ ID Nos:46-56. In certain embodiments, the C-terminal or N-terminal 1, 2, 3, 4, 5, 6, 7, 8, 9, or 10 residues of any of the sequences specified in Table 2 and Table 1B SEQ ID NOs: 35-56 may be truncated or removed.

Although recognition of the target peptide in the context of HLA is required for efficacy, for safety purposes, in some embodiments it is preferred that the TCR lacks cross-reactivity with structurally similar peptides when presented by HLA-A*02:01 or with HLA molecules of other allotypes. The cross-reactivity and alloreactivity of the exemplary TCRs described herein are provided in the Examples. Thus, the exemplary TCRs not only are able to recognize the MAGE-B2 peptide in the context of HLA-A*02:01 as expressed on tumor cells and activate a T cell recombinantly expressing the TCR against the tumor cell but also fail to activate or have minimal activation when the recombinant T cell is presented with peptides in the context of HLA-A*02:01 or other HLA molecules that are expressed on normal tissue.

Further embodiments of the present invention include nucleic acids encoding a TCR alpha variable domain, a TCR beta variable domain, or a TCR alpha variable domain and a TCR beta variable domain described herein. In particular embodiments, the nucleic acid encodes one or more of the alpha or beta variable domains set forth in Table 2. In certain embodiments, the nucleic acid encodes both alpha and beta variable domains of TCR1, TCR2, TCR3, TCR4, TCR5, TCR6, TCR7, TCR8, TCR9, TCR10, or TCR11. In preferred embodiments, the nucleic acid encoding the TCR alpha chain variable domain, TCR beta chain variable domain, or TCR alpha chain variable domain and beta chain variable domain is an expression vector wherein the TCR alpha chain variable domain, TCR beta chain variable domain, or TCR alpha chain variable domain and beta chain variable domain is operably linked to a promoter.

The TCR alpha variable domain and beta variable domain may be co-transcribed from the same promoter. For embodiments wherein the alpha variable domain and beta variable domain are linked within a fusion protein, the domains may be co-translated within a single polypeptide as well. In embodiments wherein the alpha domain and beta domain are within separate polypeptides, it is useful to include an internal ribosome entry site (IRES) between the alpha variable domain and beta variable domain coding regions within the expression vector.

Also provided herein are nucleic acids encoding a TCR alpha chain, a TCR beta chain, or a TCR alpha and TCR beta chain described herein. In particular embodiments, the nucleic acid encodes one or more of the alpha or beta chains set forth in Table 1. The encoded alpha or beta chain may be full-length or mature. When mature, i.e., lacking the nature leader sequence associated with that alpha or beta chain, it is preferred that a nucleic acid encoding a signal or leader sequence is operably connected to the nucleic acid encoding the alpha chain or beta chain such that, when translated, the leader sequence directs the alpha or beta chain to the endoplasmic reticulum.

In certain embodiments, the nucleic acid encodes both alpha and beta chains of TCR1, TCR2, TCR3, TCR4, TCR5, TCR6, TCR7, TCR8, TCR9, TCR10, or TCR11. In preferred embodiments, the nucleic acid encoding the TCR alpha chain, TCR beta chain, or TCR alpha chain and beta chain is an expression vector wherein the TCR alpha chain, TCR beta chain, or TCR alpha chain and beta chain is operably linked to a promoter.

The TCR alpha chain and beta chain may be co-transcribed from the same promoter. In such embodiments, it is useful to include an internal ribosome entry site (IRES) between the alpha chain and beta chain coding regions within the expression vector.

The expression vectors of the present invention include, but are not limited to, retroviral or lentiviral vectors. The expression vector may further encode one or more additional proteins besides the TCR alpha chain and/or beta chain. In certain embodiments, the expression vector encodes one or more cytokines. In preferred embodiments, the cytokine is a T cell growth factor such as IL-2, IL-7, IL-12, IL-15, IL-18, or IL-21, along with combinations thereof. Because cytokines can have systemic effects, when the expression vector encoding the cytokine is used to produce a cell for adoptive cell therapy, it is preferred that the cytokine expression is controlled by an inducible promoter. In certain embodiments, the promoter is a composite promoter containing six NFAT (nuclear factor of activated T cells) response elements linked to a minimal IL-2 promoter and the cytokine is IL-12 or a variant thereof. Use of a composite promoter containing six NFAT (nuclear factor of activated T cells) response elements linked to a minimal IL-2 promoter to express IL-12 is described in U.S. Pat. No. 8,556,882.

Provided herein are cells recombinantly expressing an exemplary TCR described herein. Said recombinant cells may comprise one or more expression vectors encoding and expressing a TCR alpha chain, a TCR beta chain, a TCR alpha and beta chain, a TCR alpha variable domain, a TCR beta variable domain, or TCR alpha and beta variable domains. In preferred embodiments, the cell recombinantly expresses TCR1, TCR2, TCR3, TCR4, TCR5, TCR6, TCR7, TCR8, TCR9, TCR10, or TCR11. In certain embodiments, the cell further expresses one or more recombinant cytokines. In preferred embodiments, the cytokine is IL-12 or a variant thereof and said expression is controlled by an inducible promoter, e.g., an NFAT driven promoter.

In certain embodiments, the cells are derived from a sample taken from a cancer patient. Cells, such as T cells, NKT or NK cells, are isolated from the sample and expanded. In certain embodiments, progenitor cells are isolated and matured to the desired cell type. The cells are transfected/transformed with one or more vectors, e.g., lentiviral vectors, encoding the components of the TCR along with any additional polypeptides, e.g., IL-12 or a variant thereof. Such cells may be used for adoptive cell therapy for the cancer patient from whom they were derived.

In other embodiments, a cell line recombinantly expresses a soluble TCR. The soluble TCR may be a fusion protein with an anti-CD3 antigen binding protein such as an scFv.

Provided herein are methods of treating a disease or disorder wherein cells associated with the disease or disorder express MAGE-B2 and/or MAGE-A4. In preferred embodiments, the cells present the MAGE-B2 derived peptide GVYDGEEHSV and/or the MAGE-A4 peptide GVYDGREHTV in the context of an HLA class I molecule, preferably HLA-A2, particularly HLA-A*02:01. Exemplary diseases or disorders that may be treated with the soluble TCRs or recombinant cells of the present invention include hematological or solid tumors. Such diseases and disorders include, but are not limited to, lung cancer, ovarian cancer, squamous cell lung cancer, melanoma, breast cancer, gastric cancer, testicular cancer, head and neck cancer, uterine cancer, esophageal cancer, bladder cancer, and cervical cancer. Preferred diseases and disorders include non-small cell lung cancer (NSCLC), head and neck squamous cell carcinoma (HNSCC), bladder cancer, esophageal cancer, or ovarian cancer.

For certain treatments, a biopsy of the tumor is tested for expression of MAGE-B2 or MAGE-A4. The tumor may also be tested for expression of an appropriate HLA molecule that is recognized by a TCR of the present invention when presenting the MAGE-B2- or MAGE-A4-derived peptide. Patients whose tumors express MAGE-B2 or MAGE-A4 and are of the appropriate HLA haplotype may be administered a soluble TCR or recombinant cell of the present invention.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. As used herein, “can be” or “can” indicates something envisaged by the inventors that is functional and available as part of the subject matter provided.

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. All documents, or portions of documents, cited in this application, including but not limited to patents, patent applications, articles, books, and treatises, are hereby expressly incorporated by reference.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

The following examples, both actual and prophetic, are provided for the purpose of illustrating specific embodiments or features of the present invention and are not intended to limit its scope.

### Example 1—MAGE-A4 and MAGE-B2 are Expressed Across a Broad Range of Solid Tumors with Highly Restricted Normal Tissue Expression

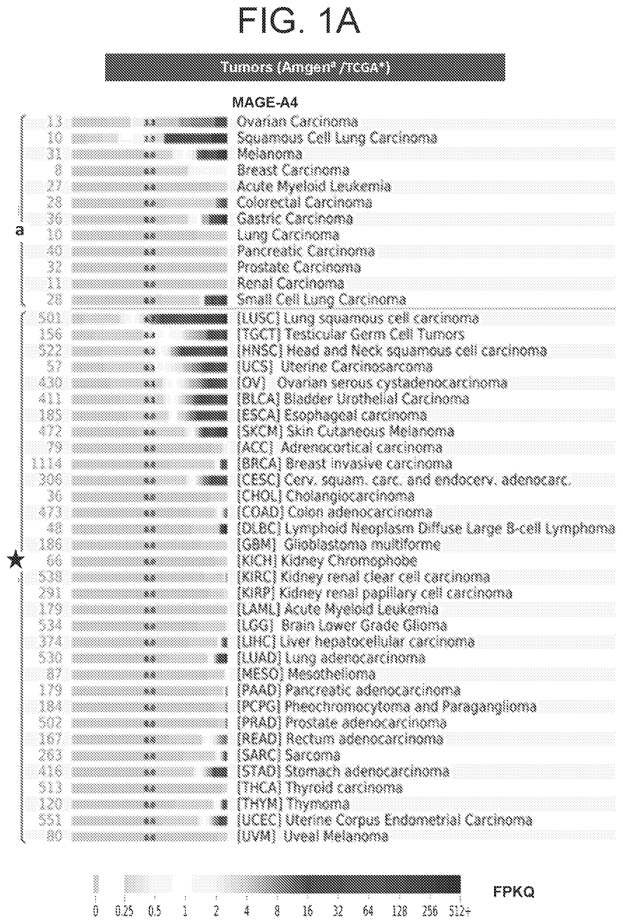

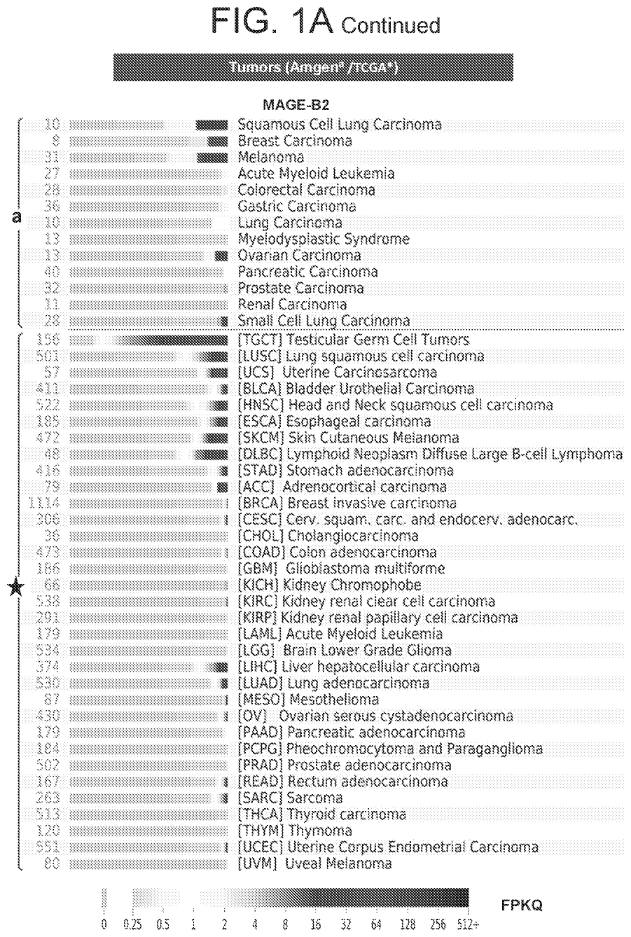

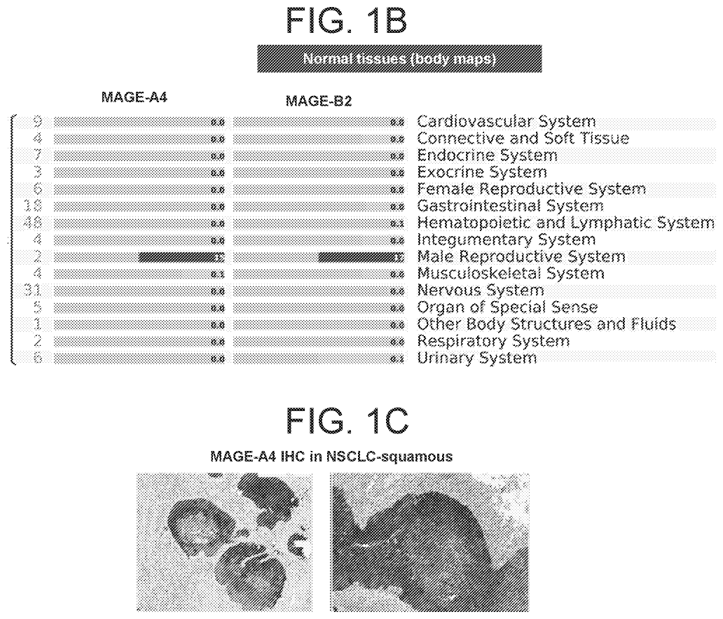

The Cancer Genome Atlas (TCGA) and Applicant's data demonstrate that MAGE-A4 and MAGE-B2 mRNA have high prevalence across a broad range of solid tumors (FIG. 1A). Importantly, Applicant's internal body map data show extremely restricted normal tissue expression of MAGE-A4 and MAGE-B2 mRNA, except testis, which is an immune privileged site (FIG. 1B). The MAGE-A4 IHC data in NSCLC-squamous (squamous non-small cell lung cancer or lung squamous cell carcinoma) shows within a tumor, MAGE-A4 protein is expressed in the majority of tumor cells (60-100%), and not in stromal cells (FIG. 1C). Similarly, the MAGE-B2 ISH data shows that within a tumor, MAGE-B2 mRNA is expressed in the majority of NSCLC tumor cells (>50%), and not in stromal cells (data not shown).

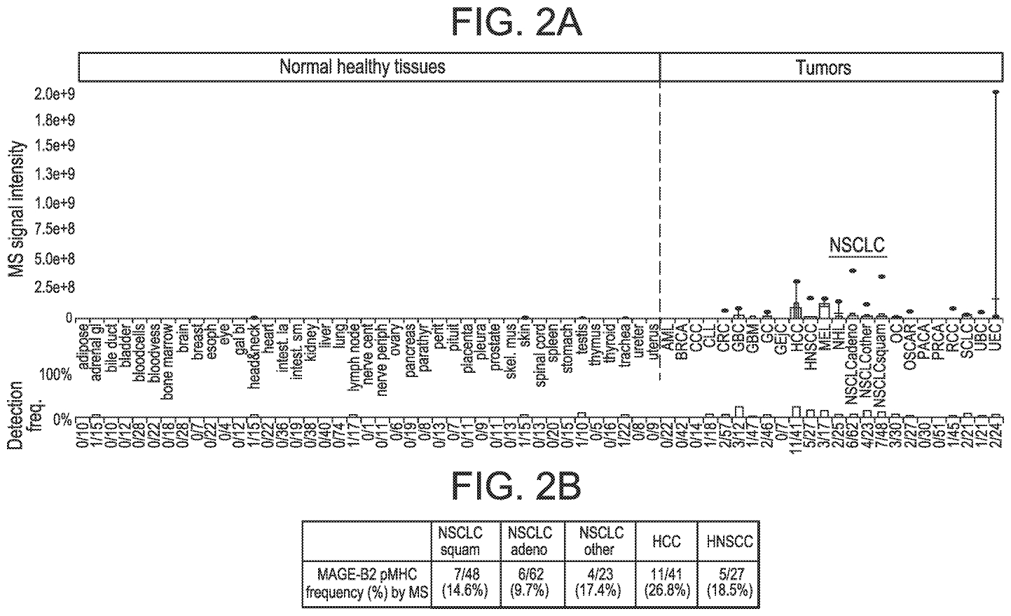

Furthermore, as pMHC targets, MAGE-B2 and MAGE-A4 peptide presentation on HLA-A*02:01 were validated by mass spectrometry (MS). The MS data using various tumors and normal tissues (Immatics, Tuebingen, Germany) demonstrated that MAGE-B2 peptide-MHC (GVYDGEEHSV/HLA-A*02:01) expression is very specific for tumors, not detected in normal healthy tissues (FIG. 2A). The MAGE-B2 pMHC frequencies in representative cancer types measured by MS are shown in the table (FIG. 2B). The MAGE-B2 peptide GVYDGEEHSV (SEQ ID NO:1) corresponds to amino acid residues 231-240 of MAGE-B2 protein. In addition, in-house MS data confirms MAGE-A4 pMHC expression in squamous NSCLC tumors (data not shown). The MAGE-A4 peptide GVYDGREHTV (SEQ ID NO:2) corresponds to amino acid residues 230-239 of MAGE-A4 protein.

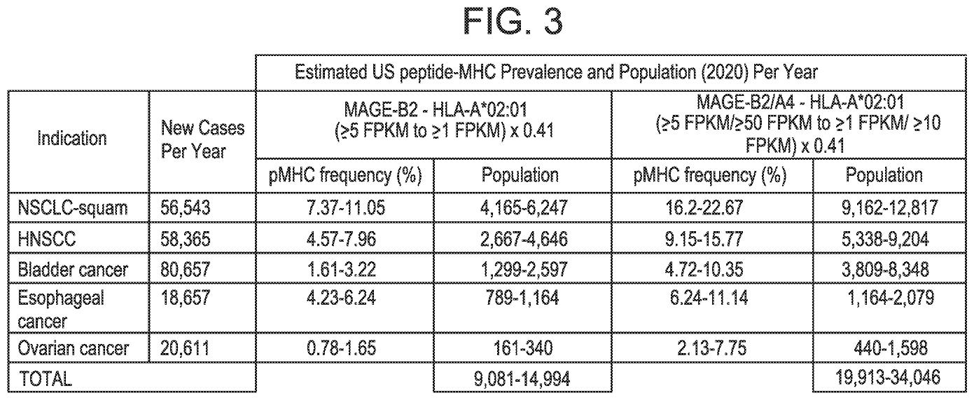

MAGE-A4 and MAGE-B2 are expressed in a wide range of cancer types. The solid tumor indications with MAGE-B2 and/or MAGE-A4 pMHC expression (MAGE-B2/A4-HLA-A*02:01) include, but are not limited to, 16.2-22.7% of lung squamous cell carcinoma (NSCLC-squamous, LUSC), 9.2-15.8% of head and neck squamous cell carcinoma (HNSCC), 6.2-11.1% of esophageal carcinoma, 4.7-10.4% of bladder cancer, and 2.1-7.8% of ovarian cancer (FIG. 3). The patient population in specified cancer indication was estimated based on pMHC target frequency (%) multiplied by new cases (new patient number) per year in U.S. populations. The pMHC target frequency (%) was calculated by MAGE-B2 and/or MAGE-A4 mRNA expression frequency multiplied by HLA-A*02:01 carrier frequency in the U.S (0.41). The TCGA public datasets of RNAseq from tumors of interest were used to estimate MAGE-B2 and/or MAGE-A4 mRNA expression frequency in each tumor indication at a threshold of (1) MAGE-B2>=1 FPKM and/or MAGE-A4>=10 FPKM or (2) MAGE-B2>=5 FPKM and/or MAGE-A4>=50 FPKM (FIG. 3). Patients positive for both MAGE-B2 and MAGE-A4 targets were not counted twice. SEER, EPIC Oncology New Patients, or Epiphany/Epic in 2020 was used to estimate disease incidence (new cases per year) in selected tumor indications and hence derive estimated treatable patient population ranges (FIG. 3). HLA-A*02:01 is one of the most common MHC class I alleles in U.S. The HLA-A*02:01 haplotype (carrier) frequency estimate in U.S. populations is 0.41 (www.allelefrequencies.net). The US patient populations double when both MAGE-A4 and MAGE-B2 are covered, compared to MAGE-B2 alone. The largest patient population is in NSCLC-squamous, followed by HNSCC, bladder cancer, esophagus cancer, and ovarian cancer (FIG. 3).

### Example 2—Identification of MAGE-B2 pMHC-Specific TCRs

The process to identify and select lead clinical TCR candidates is outlined in below. First, using a TCR discovery platform based on ex vivo stimulation and scRNAseq, 40 dominant MAGE-B2 pMHC-specific TCRs were identified using 52 healthy HLA-A*02:01+ donors. Using Jurkat activation assays, 11 TCR candidates were selected from 40 TCRs. Based on these 11 TCR sequences, 11 TCR-T cells per donor were generated by transduction of primary pan-T cells isolated from 3 donors with lentivirus carrying individual TCRs. Those TCR-T cells were further evaluated by various functional assays including potency (cytotoxicity) tests with T2 cell line that were pulsed with target peptides and multiple (˜20) cancer cell lines, cross-reactivity screen with similar peptides, and initial alloreactivity screen. Based on the functional data, we narrowed down to top 4 TCR candidates out of 11 TCRs. To further enhance the in vivo efficacy and decrease clinical doses, the top 4 TCRs were manufactured in a TCR-T-IL12 lentiviral construct, where the IL12 payload expression is induced upon by TCR activation under a NFAT response-driven promoter. Therefore, only when TCR-T cells bind to the pMHC targets (MAGE-B2 and/or MAGE-A4) in tumors, the IL12 can be produced. The TCR-T-IL12 cells generated from 3 donors were further evaluated by various functional assays, including potency tests with T2 cell line pulsed with target peptides and multiple (˜40) cancer cell lines, cross-reactivity with full panel similar peptides, normal cell cytotoxicity screen, and full alloreactivity screen. Based on all the data from these evaluations, we selected one lead clinical TCR candidate.

MAGE-B2 pMHC-Specific TCRs can be Identified from Rare T Cell Clones Isolated from Healthy Donor PBMCs

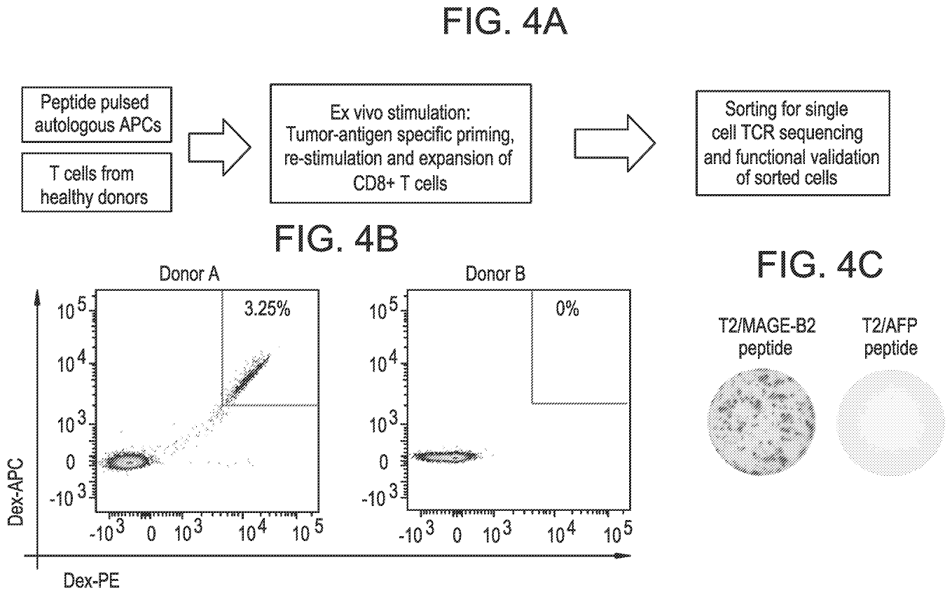

Difficulties in identifying tumor antigen-specific TCRs have hampered the development of TCR-mediated immunotherapies. Despite these challenges, we have successfully developed a TCR discovery platform by which the tumor antigen pMHC-specific TCRs can be identified from rare T cell clones isolated from healthy donors PBMCs (FIG. 4A). The frequencies of MAGE-B2 pMHC-reactive T cells in PBMCs from healthy HLA-A*02:01+ donors were extremely low, which were typically ˜0% dextramer+ T cells. DEXTRAMER® (Dex) is a multimer of peptide-MHC complexes that can specifically bind to TCRs, and therefore can be used to isolate antigen (pMHC)-specific T cells. First, in order to expand the rare tumor antigen-specific T clones, we used 52 healthy HLA-A*02:01+ donor's PBMCs to isolate T cells and autologous antigen-presenting cells (APCs) such as monocyte-derived dendritic cells and activated B cells. Upon co-culture of T cells with the autologous APCs pulsed with target peptides, these T cells went through multiple steps of ex vivo stimulations where tumor antigen pMHC-specific priming, restimulation, and expansion of pMHC-specific T cells occur. After multiple antigen restimulations, a population of MAGE-B2 pMHC dextramer+ (Dex+) T cells (MAGE-B2 pMHC-reactive T cells) were detected. After 2-4 rounds of antigen restimulations, the MAGE-B2 pMHC-specific T cell population was more enriched and validated by both Dextramer-PE and dextramer-APC stains (FIG. 4B). The Dex+CD8+ T cells were then sorted for single cell RNAseq to identify the sequences of TCRα and TCRβ chains. The SEQ ID Numbers corresponding to the TCRα and TCRβ sequences of representative TCRs identified are listed in Table 1. Furthermore, those sorted Dex+CD8+ T cells were validated for MAGE-B2 antigen-specific activation by an IFNγ ELISPOT assay using peptide-loaded T2 cells (FIG. 4C). This TCR discovery platform led to the identification of 40 dominant MAGE-B2 pMHC-specific TCRs from 52 healthy HLA-A*02:01+ donors. Importantly, the TCRs identified from healthy donor blood have been through thymic natural selection in the human body (in the medulla of the thymus) to eliminate self-reactive TCRs, unlike affinity-enhanced TCRs or bispecific antibodies. Therefore, it is contemplated that the risk of off-targets for the TCRs is fairly low, which was confirmed by safety assessment assays (described below).

Selection of Top MAGE-B2 pMHC-Specific TCR-T Cells

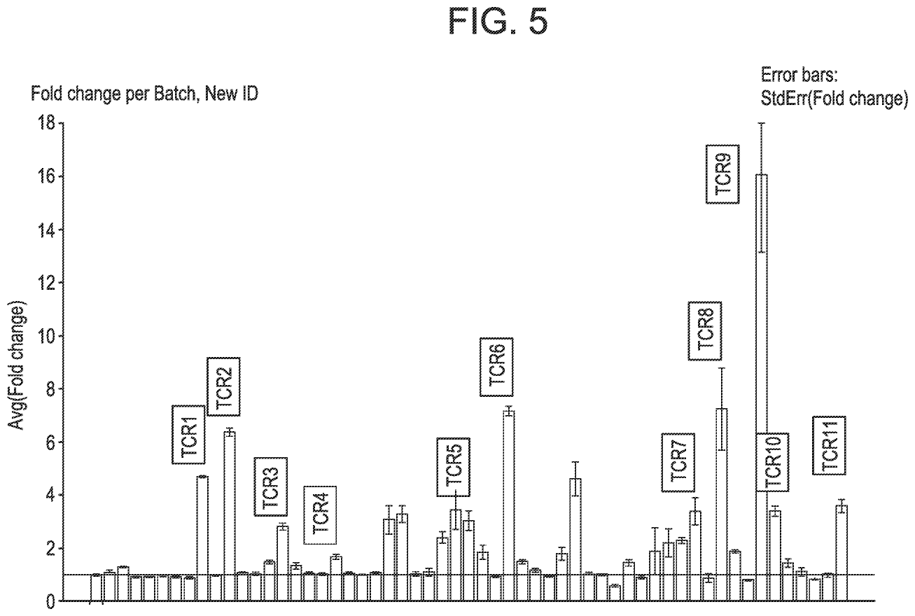

Out of 40 dominant MAGE-B2 pMHC-specific TCRs identified from a screen of 52 healthy HLA-A*02:01+ donors, 11 TCR candidates were selected by a Jurkat activation assay (FIG. 5). Lentivirus carrying individual TCRs were transduced into a Jurkat TCR KO reporter cell line expressing CD8a constitutively and Renilla luciferase that is regulated by TCR activation under a NFAT response element driven promoter. The activity of individual TCR was measured as the fold change of the luciferase activity in the presence of T2 cells loaded with the MAGE-B2 peptide compared to T2 cells with vehicle only (FIG. 5).

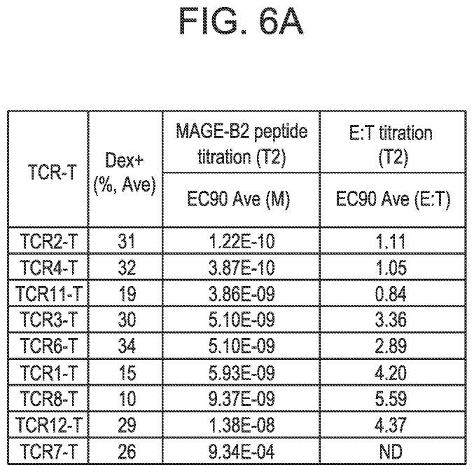

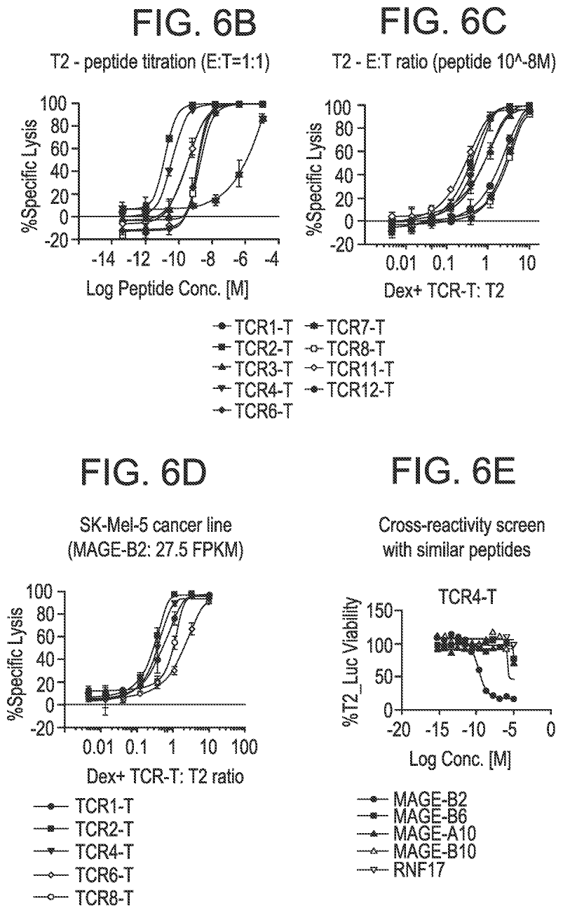

Based on these eleven selected TCR sequences, eleven TCR-T cell lines per donor were generated by transducing human primary pan-T cells isolated from three donors with lentivirus carrying individual TCRs. Those TCR-T cells were further evaluated by various functional assays. First, the potency of each TCR-T was assessed by using T2/peptide cytotoxicity assays (MAGE-B2 peptide) including peptide titration and E:T (effector:target cell ratio) titration assays (FIG. 6A-6C). As T2 is a cell line deficient in the transporter associated with antigen processing (TAP) and expresses HLA-A*02, MHC class I-restricted endogenous peptides are unable to enter the ER and the T2 cell line presents mainly exogeneous peptides. Therefore, the T2/peptide cytotoxicity assay (cytolytic activity measurement using T2 cell line loaded by a peptide of interest) was used to study the specific recognition of peptides (e.g. HLA-A*02:01-restricted) by TCRs of T cells. The potencies of two TCRs, TCR2-T and TCR4-T, against T2/MAGE-B2 peptide were very similar (FIG. 6A-6C). Importantly, TCR3-T and TCR4-T were also cross-reactive to MAGE-A4 peptide, which is also a cancer testis antigen with high prevalence in a broad spectrum of solid tumors as described before. TCR4-T showed much higher potency to MAGE-A4 peptide compared to TCR3-T. In addition, cytotoxicity against multiple (˜20) MAGE-B2+ and/or MAGE-A4+ cancer cell lines were evaluated. Representative cytotoxicity against SK-MEL-5 line is shown in FIG. 6D. Exemplary TCR-Ts displayed potent killing activities against cancer cell lines with MAGE-B2 expression as low as ˜1.4 FPKM or E:T EC50 as low as ˜0.25.

To assess off-target selectivity, TCR-T cells were examined by the T2/peptide cytotoxicity assay using 131 homology-based similar peptides and target negative cancer lines. Representative data are shown in FIG. 6E. The details of off-target strategy and identification of similar peptides are described below.

For an initial alloreactivity, TCR-Ts were tested in co-culture with 5 B lymphophoblastoid cell lines (BLCLs) representing the top 5 most frequent non-HLA-A*02:01 alleles in the US population (e.g. HLA-A*01:01, HLA-A*03:01, HLA-A*11:01, HLA-A*24:02, HLA-A*02:07). IFNγ and granzyme B production were used as readouts for initial alloreactivity. The details of alloreactivity are described below.

Top four TCRs (TCR1, TCR2, TCR3, TCR4) were selected out of the eleven TCRs, based on various functional studies including (1) potent cytotoxicity on MAGE-B2 and/or MAGE-A4 pMHC targets, using T2/MAGE-B2 peptide, T2/MAGE-A4 peptide and ˜20 MAGE-B2+ and/or MAGE-A4+ cancer cell lines, (2) off-target selectivity showing no cross-reactivity against 131 homology-based similar peptides and target negative cancer cell lines, (3) no initial alloreactivity, and (4) manufacturability (e.g. good TCR transduction efficiency).

### Example 3—Potency Validation of TCR-T-IL12 Cells

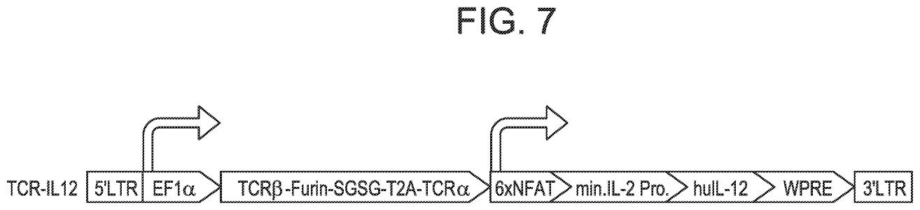

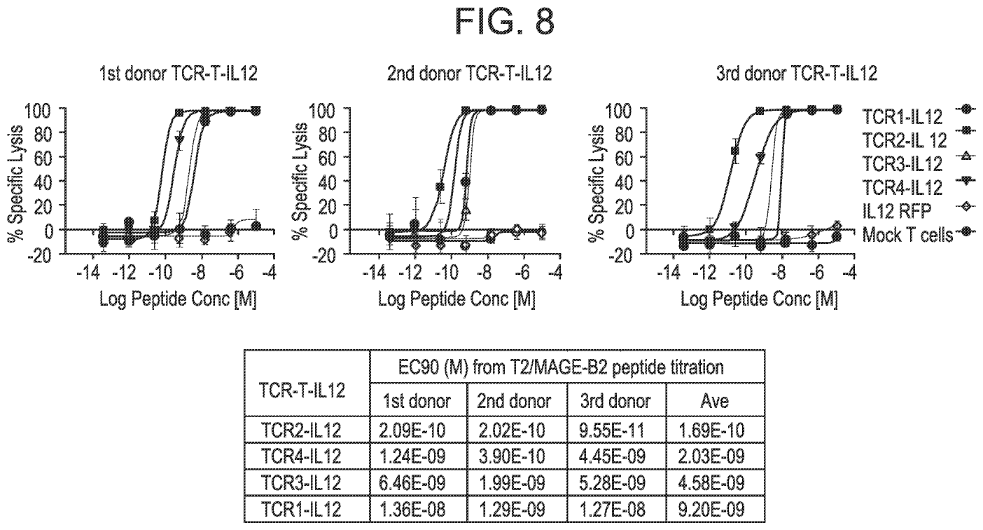

The top four TCRs selected by various functional assays described above were further manufactured in a TCR-T-IL12 lentiviral construct, where the IL12 payload expression is regulated by TCR activation under an NFAT response element driven promoter (FIG. 7). Therefore, when TCR-T-IL12 cells bind to the pMHC targets (MAGE-B2 and/or MAGE-A4) in tumors, the IL12 is produced upon TCR signaling. The TCR-T-IL12 cells generated using three donors were further evaluated by various functional assays. First, potency validation was conducted using the T2/MAGE-B2 peptide cytotoxicity assay (FIG. 8). The potency ranking of the four TCR-T-IL12s remains the same as that of parental TCR-Ts (without IL12). TCR2-IL12 showed the highest potency followed by TCR4-IL12, TCR3-IL12, and then TCR1-IL12 as the lowest. All four TCR-IL12 cells met a potency criterion with EC90 of 10⁻⁸M (peptide concentration) by T2/peptide cytotoxicity assay.

### Example 4—Potent Cytotoxicity of TCR4-IL12 Against Both MAGE-B2 Peptide- and MAGE-A4 Peptide-Loaded T2 Cells

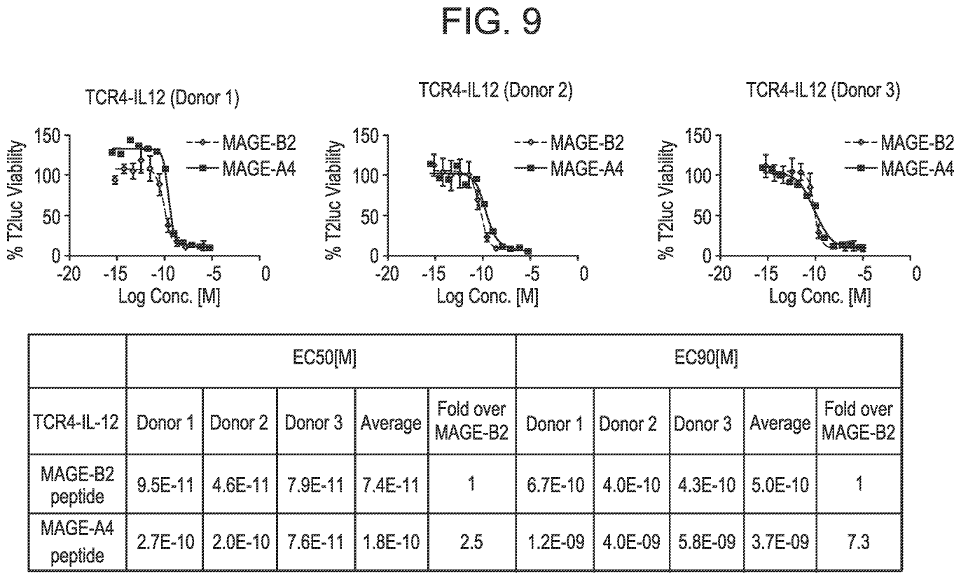

Notably, TCR4-IL12 can also recognize MAGE-A4 peptide MHC with high potency in T2/peptide cytotoxicity assay (FIG. 9). Potency gaps between MAGE-A4 and MAGE-B2 peptides for this TCR-T-IL12 were only 2.5-fold for EC50 and about 7-fold in EC90. The potency data from three different donors showed that the potencies against MAGE-B2 and MAGE-A4 peptides are quite similar (graphs in FIG. 9). Importantly, TCR4-IL12 met a potency criterion with EC90 of 10⁻⁸M (peptide concentration) for both MAGE-B2 and MAGE-A4 peptides.

### Example 5—Cytotoxicity Against MAGE-B2+ and/or MAGE-A4+ Cancer Cell Lines

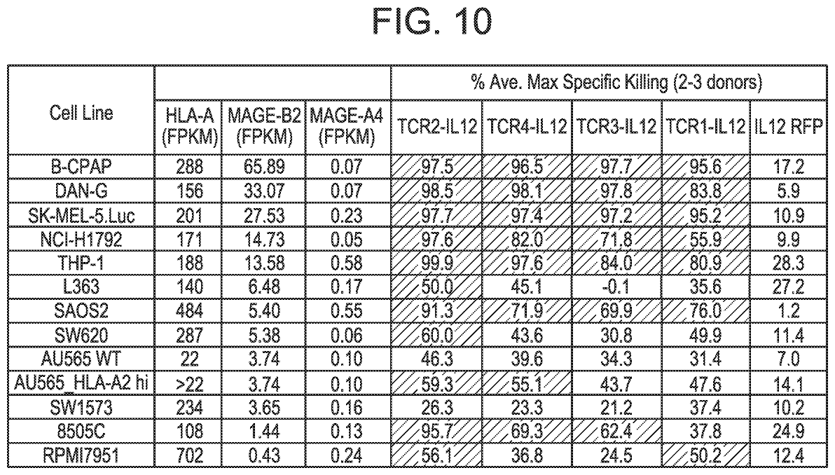

The potencies (cytotoxicity) of the four TCR-T-IL12 were validated using three different categories of cancer cell lines, including MAGE-B2+ MAGE-A4−, MAGE-B2− MAGE-A4+, and MAGE-B2+ MAGE-A4+ cancer cell lines. First, the potency of TCR-T-IL12 was assessed by using MAGE-B2+ MAGE-A4− cancer cell lines (FIG. 10). All four TCR-T-IL12s displayed potent cytotoxicity against cancer cell lines with MAGE-B2 expression as low as ˜1.4 FPKM. In potency ranking assays against MAGE-B2+ MAGE-A4− cancer cell lines, TCR2 was the most potent TCR, followed by TCR4, and then TCR1 and TCR3 were similar. TCR2-IL12 and TCR4-IL12 displayed cytotoxicity at E:T EC50 as low as ˜0.07 and 0.21, respectively. The TCR-T-IL12 showed the high potency against even MAGE-B2 low cancer cell lines such as 8505C (˜1.4 FPKM) and AU565 HLA-A2hi (˜3.7 FPKM). The target-specific killing against these MAGE-B2-low cancer cell lines was verified by MAGE-B2 KO cell lines generated from these low cancer cell lines (described below).

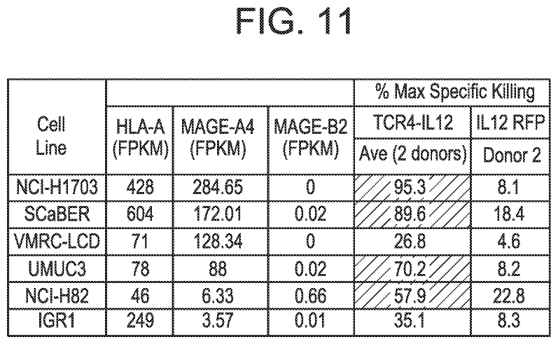

Second, the potency of TCR4-IL12 against MAGE-A4+ MAGE-B2− cancer cell lines were accessed given the cross-reactivity of this TCR to MAGE-A4 peptide from T2/peptide assay (FIG. 11). TCR4-IL12 showed cytotoxicity against cancer cell lines with MAGE-A4 expression as low as ˜6.3 FPKM or E:T EC50 as low as ˜0.46.

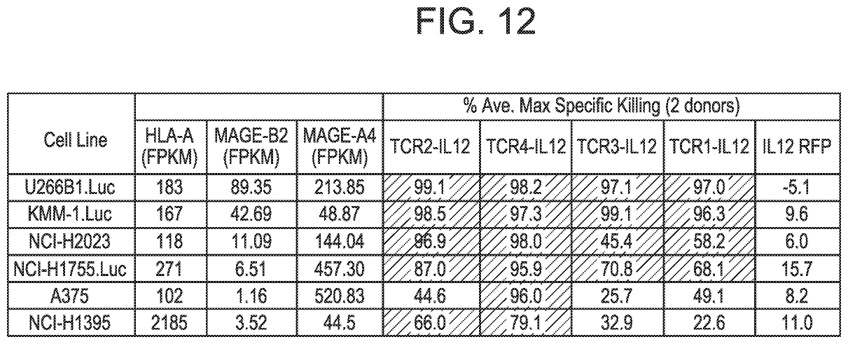

Third, the potency against double-positive, MAGE-B2+ MAGE-A4+ cancer cell lines was evaluated (FIG. 12). TCR4-IL12 and TCR2-IL12 showed potent cytotoxicity against MAGE-B2+ MAGE-A4+ cancer cell lines. In potency ranking against MAGE-B2+ MAGE-A4+ cancer cell lines, TCR4 was the most potent TCR, followed by TCR2, and then TCR3 and TCR1 were similar. Notably, TCR4-IL12 showed the highest potency against the double-positive MAGE-B2+ MAGE-A4+ cancer cell lines due to high potency to both MAGE-B2 and MAGE-A4 peptides. Particularly, some MAGE-B2-low MAGE-A4-hi cancer cell lines (e.g. A375) can differentiate potency between TCR4 and TCR2 as TCR2 has only MAGE-B2 specificity without MAGE-A4 cross-reactivity. TCR4-IL12 demonstrated cytotoxicity against the double positive cancer cell lines with MAGE-B2 expression as low as ˜1.2 FPKM or MAGE-A4 expression as low as ˜44 FPKM, or E:T EC50 as low as 0.04. TCR2-IL12 displayed cytotoxicity against cell lines with MAGE-B2 expression as low as ˜3.5 FPKM or E:T EC50 as low as 0.01.

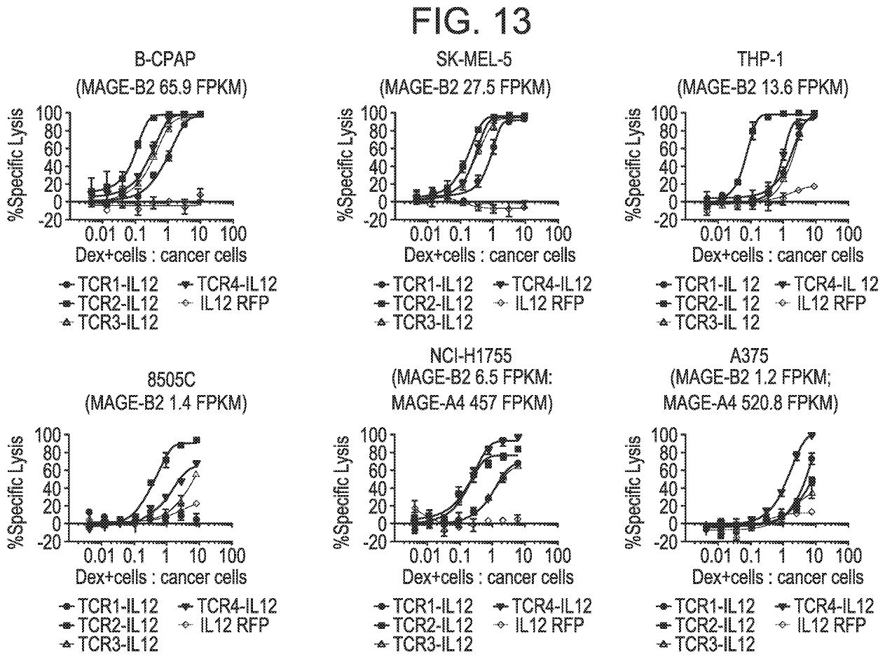

Representative cancer cell line potency data of the four TCR-T-IL12 cells are shown in FIG. 13. About 40 cancer cell lines were tested with four TCR-T-IL12 cells generated from 2-3 donors. TCR-T-IL12 cells demonstrated potent cytotoxicity against some cancer cell lines with low E:T EC50. For example, E:T EC50 of TCR4-IL12 against cancer cell lines were 0.21 for B-CPAP, 0.25 for SK-MEL-5, 0.98 for THP-1 and 0.25 for NCI-H1755.

### Example 6—Peptide-MHC Target-Specific Cytotoxicity Validation by MAGE-B2 KO and B2M KO Cancer Cell Lines

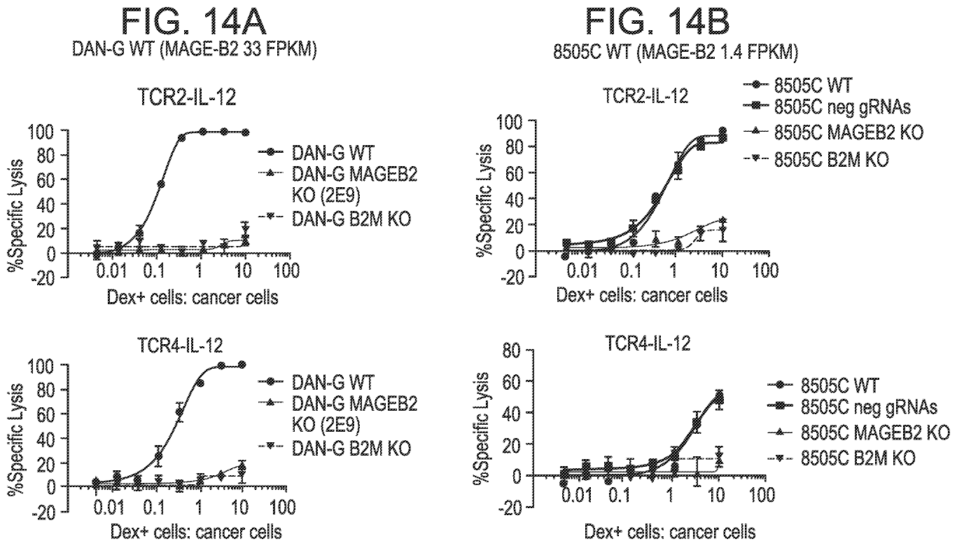

As potent cytotoxicity of TCR-T-IL12 against multiple MAGE-B2+ cancer lines with very low expression of MAGE-B2 was observed, it was determined if this cytotoxicity depends on the pMHC target expression. Hence, we generated MAGE-B2 KO (knockout) cell lines and B2M KO cell lines to eliminate the expression MAGE-B2 and B2M respectively (FIGS. 14A and 14B). B2M (B2 microglobulin) is a critical subunit of MHC class I molecules. Both MAGE-B2 KO and B2M KO resulted in the loss of killing activity by either TCR2-IL12 or TCR4-IL12. Remarkably, upon KO of MAGE-B2 in 8505C cancer cell line that has very low MAGE-B2 mRNA expression (1.4 FPKM), both TCR-T-IL12 cells lost the killing ability, indicating that the cytolytic activity of TCR-T-IL12 cells depends on the MAGE-B2 target expression and TCR-T-IL12 can truly recognize such a low expression of the target (FIG. 14B). Similarly, loss of cytotoxicity was seen in B2M KO lines, demonstrating that TCR-T-IL12 activities reply on HLA expression.

### Example 7—Effect of IL12 Payload on TCR-T Potency

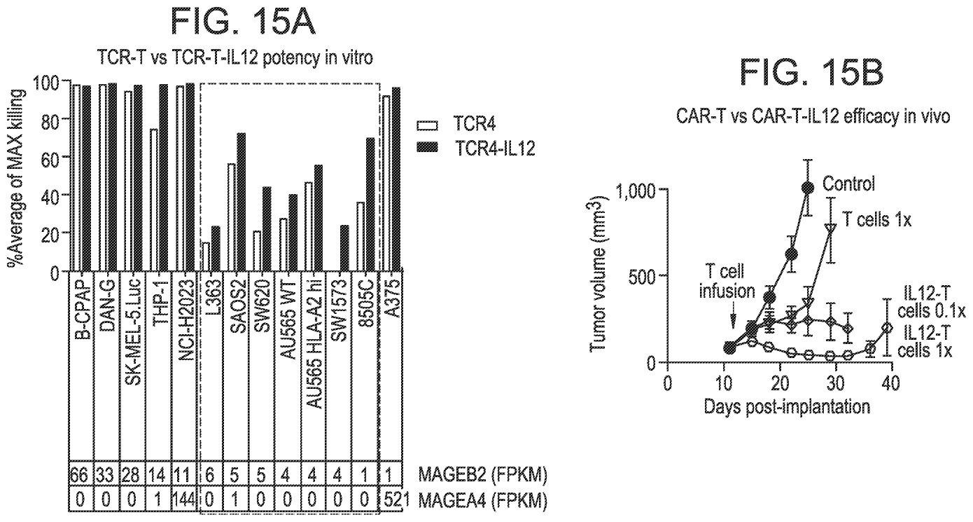

HuEpCAM CAR-T cells with or without IL12 payload were assessed in a B16F10-huEpCAM syngeneic mouse tumor model. This mouse study demonstrates that IL12 payload enhances T cell efficacy in vivo and could decrease potential clinical dose (FIG. 15B).

Next, we assessed the effect of IL12 payload in a human TCR-T system with multiple cancer cell lines. Particularly for MAGE-B2-low cancer cell lines (shown inside the dotted line box), the IL12 payload can increase TCR-T cell potency, compared to parental TCR-Ts without IL12 (FIG. 15A). For MAGE-B2-high or MAGE-A4-high cancer cell lines (shown outside the dotted line box), because the potency was already maxed out by parental TCR-T, there was not much effect of IL12 for those cancer cell lines.

### Example 8—Overview of Nonclinical Safety Assessment

An extensive in vitro and ex vivo safety assessment for TCR-T-IL12 cells was performed, as the human-specific HLA target precludes the use of animal models. First, the target expression was assessed by various assays including RNASeq, IHC, and mass spectrometry using normal human tissues as well as tumor tissues, which were described above. As MAGE-B2 and MAGE-A4 are cancer testis antigens, the studies displayed extremely restricted normal tissue expression (only expressed in testis). Second, off-target reactivity was assessed which were assessed using two different strategies. The first strategy involved evaluating cytotoxicity against various normal human primary cell types representative of major organs. The second strategy involved identifying a panel of similar peptides based on sequence homology match to the MAGE-B2 target peptide along with a positional scanning (X-scan)-based strategy to identify putative cross-reactive peptides unique to each TCR. To assess potential cross-reactivity to this panel of similar peptides T2/peptide TECC assays were conducted. The third safety assessment was alloreactivity, which was assessed using 34 BLCLs representing highly frequent HLA class I alleles in US populations, including 38 HLA-A, 40 HLA-B and 24 HLA-C alleles.

### Identification of Similar Peptides Based on Homology

To assess off-target reactivity, a full panel of similar peptides to MAGE-B2 target peptide were identified using two different strategies, based on either sequence homology to target peptide or X-scan-derived motifs.

A homology-based strategy was designed using an in-silico approach to identify a list of peptides that could potentially cross-react with the candidate TCR-Ts. To accomplish this, a protein database (UniProtKB/Swiss-Prot, June 2019) query was first performed to generate a list of all possible decameric peptides, based on amino acid identity match to the target MAGE-B2 peptide (GVYDGEEHSV). This in silico query was performed using a Python script and resulted in the identification of 170,082 peptides based on 30% homology (identity) match to the target peptide. To refine this list further, criteria such as high homology match, and software such as NetMHCpan software and IEDB (The Immune Epitope Database) were utilized. NetMHCpan3.0 was used to consider a peptide's predicted binding affinity to HLA-A*02:01. IEDB database (June 2019), which is a manually curated database of experimentally characterized immune epitopes, was used to consider a peptide's chance of being processed and presented by the HLA-A*02:01 allele. Specific criteria used for peptide selection were as follows, (1) all peptides with greater than or equal to 60% homology match (identity) to the target peptide (65 peptides), (2) all peptides with greater than or equal to 50% homology match and predicted binding affinity (IC50) less than or equal to 50 nM, (35 peptides), and (3) all peptides with greater than or equal to 40% homology match to target peptide that are reported in IEDB (presented by HLAOA*02:01 allele) (45 peptides). As a result, this homology-based in silico search of human proteome database let us to the identification of 131 unique peptides.

### Identification of the TCR Binding Motif Using Positional Scanning (X-Scan) and Similar Peptides Based on X-Scan-Derived Motifs

As an orthogonal approach to identify similar peptides, we used a positional scanning method, known as X-scan. The X-scan assay uses a peptide library that is generated by sequentially mutating each residue of the MAGE-B2 peptide to one of other 19 naturally occurring amino acids, resulting in a total of 190 peptides. These 190 peptides were synthesized and tested in the T2/peptide TDCC assay to identify an X-scan derived motif that is specific to each individual TCR (Table 3). Briefly, T2 cells were pulsed with each of these peptides at a 10p M or 1p M concentration, followed by addition of TCR-T cells at an E:T ratio of 1:1. Cell viability was determined using a T2/peptide TDCC assay. An amino acid substitution was defined as essential for TCR engagement where the viability observed was less than 20%. A corresponding search motif was constructed to express which amino acids were tolerated at each position in the peptide sequence (Table 3). Underlined amino acids represent the native residue at the corresponding position in the peptide.

Using a python script, an in-silico search of the UniProtKB/Swiss-Prot database with splice variants (June 2019) was performed to identify all decameric sequences that comply with the derived motif From this motif-based blast search, unique human peptide matches, that conform to the consensus motif of the specific TCR-T, were identified.

In the case of two TCRs (TCR3 and TCR2), where the resulting motif search-based peptides were considerably large in number, further anchor residue restriction (at residues 2 and 10) was applied to the derived motif to limit final cross-reactive peptide selection (Table 3). Specifically, sequences of 2583 decameric HLA-A*02:01 positive peptides, obtained from IEDB database were analyzed to calculate the amino acid frequency at the anchor residue positions. A 3% amino acid frequency cut-off was applied to both the anchor residues (residue 2 and residue 10) of the motif, which restricted position 2 to amino acids T, M, E, I, V, L and position 10 to amino acids Y, I, A, L, V.

TABLE 3

| | | Unique peptide |
|---|---|---|
| | | matches, which |
| | | conform to the |
| TCR | Motif obtained through Positional Scanning | consensus motif |
| TCR1 | [GACDEFHILMNPQSTVWY][VACGILMST][YFW][DCENPW] | 87 |
| | [GCLMRS][EV][ECDHKLMNPQY][HFNWY][SACDEGINPQTVW] | |
| | [VACFILMT] | |
| TCR4 | [G][VIQ][YF][DCN][G][EAFHIKLMNQRSTVWY][EACDFHIL | 13 |
| | MNPQRSWY][HACDEFGIKLMNPQRSVWY][SACDEFGHIK | |
| | LMNPQRTVWY][VACFGHIKLMNSTWY] | |
| TCR3 | [G][VILMT][YF][DCN][GAS][EACFIKLMPQRSTVY][EACDF | 78 |
| | GHIKLMNPQRSTWY][HACDEFGIKLMNPQRSTVWY][SAC | |
| | DEFGHIKLMNPQRTVWY][VAILY] | |
| TCR2 | [GA][VILMT][YFW][DCNP][GP][ECDMNQSTV][EACDFHIL | 63 |
| | MNQSTWY][HACDEFGIKLMNPQRSTVWY][SACDEFGHIK | |
| | LMNPQRTVWY][VAIL] | |

Cross-Reactivity Screen with Full Panel Similar Peptides

Full panel similar peptides (including the X-scan motif-based set and homology-based set) were synthesized and examined in T2/peptide TDCC assays to investigate the likelihood of off-target reactivity.

To identify potential cross-reactive peptides for each TCR-T-IL12, the full panel of similar peptides was tested using a T2/peptide TDCC screen with a high peptide concentration (10 μM or 1 μM). Peptides that showed less than or equal to 25% viability in at least one of three donors were considered as putative cross-reactive peptides and were selected for a further potency test. All three different donors showed good agreement with peptide responses.

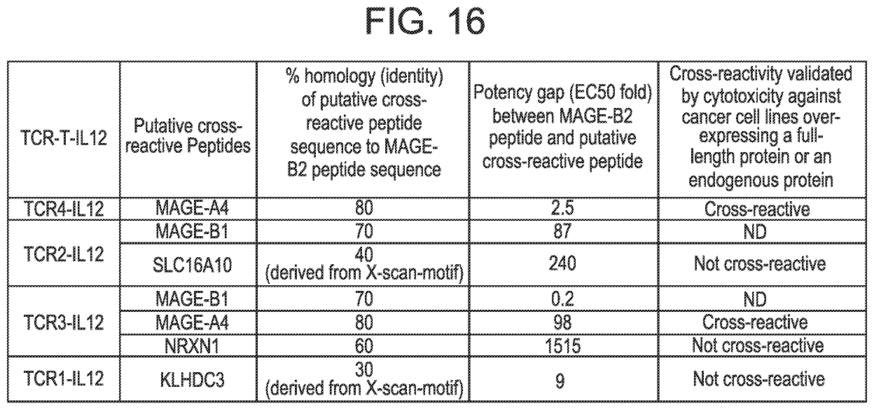

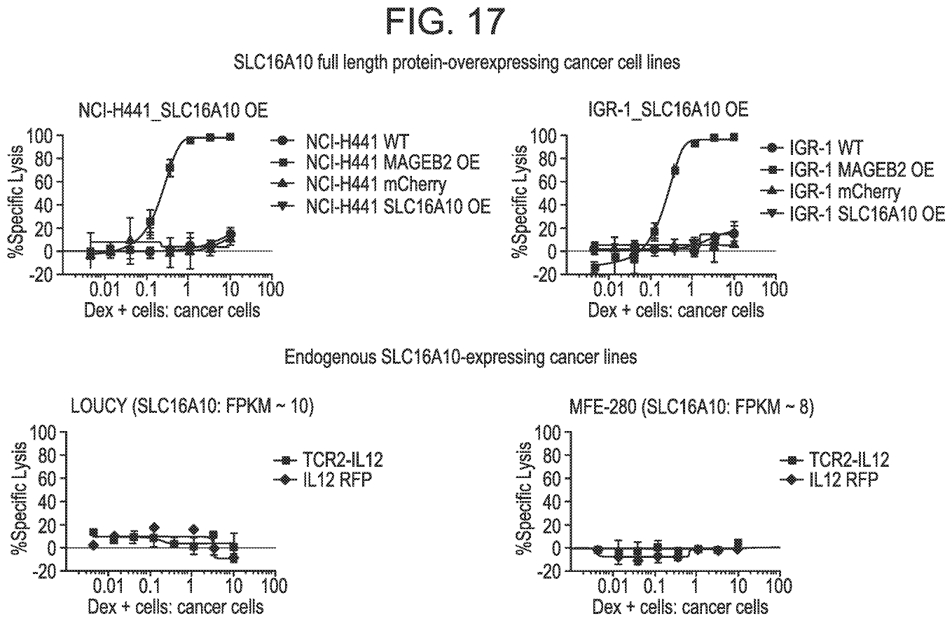

Next, a potency screen (dose dependent screen) was performed using T2/peptide titration TDCC assays for the putative cross-reactive peptides identified from the above screen. Most putative cross-reactive peptides were de-risked by this potency screen. A potency gap of less than 10³-fold in EC50 between target peptide and putative cross-reactive peptides was considered as a cutoff for further risk assessment. Results from the cross-reactivity screen with full panel similar peptides for the top four TCR-T-IL12 cells are summarized in FIG. 16, where all peptides showing less than 10³-fold potency gap to target MAGE-B2 peptide are listed. TCR4-IL12 cell did not yield any putative cross-reactive peptide (besides MAGE-A4). Each of the other three TCR-T-IL12 cells had one putative cross-reactive peptide identified from this full panel peptide screen, besides MAGE-B1, which is a cancer testis antigen. All the three putative cross-reactive peptides (arising from proteins SLC16A10, KLHDC3, and NRXN1) were further de-risked by TDCC assays with HLA-A*02:01+ cancer cell lines over-expressing the respective full length-proteins or cancer cell lines expressing the endogenous proteins (FIG. 17). No cytotoxicity against the cancer cell lines overexpressing those putative cross-reactive proteins or endogenous proteins was observed by any of those TCR-T-IL12s (TCR1, TCR2, and TCR3), suggesting that these peptides are unlikely to be naturally processed and presented from the proteins (FIGS. 16 and 17). In conclusion, none of the four TCR-T-IL12 cells demonstrated any significant cross-reactivity across the full panel of similar peptides identified by sequence homology and X-scan-derived TCR motifs.

### Assessment of Cytotoxicity Against Human Normal Cells

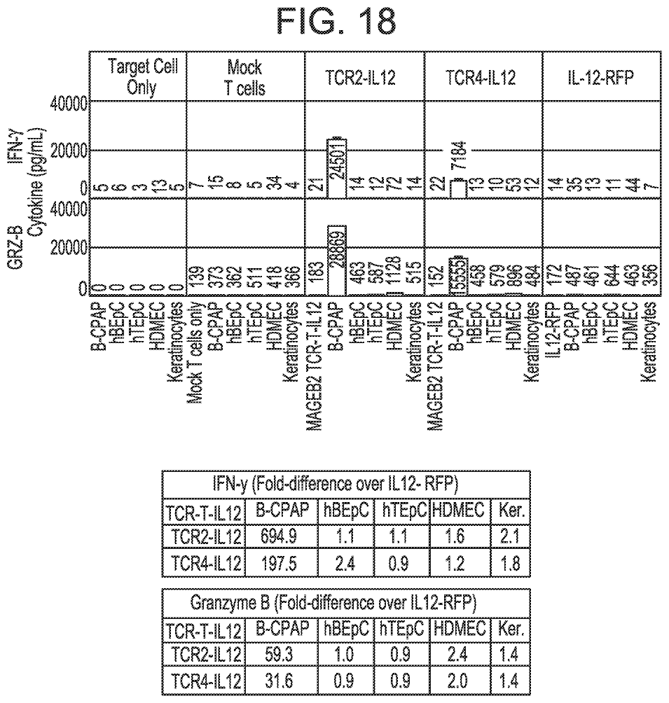

Next, the cytotoxicity of four MAGE-B2 TCR-T-IL12 cells (TCR1-IL12, TCR2-IL12, TCR3-IL12, and TCR4-IL12) was evaluated against a panel of nine normal human primary or iPSC-derived cell types representative of major organs (with no MAGE-B2 or MAGE-A4 expression) serving as target cells, in a T-cell mediated cytotoxicity assay. The panel of nine normal human cells included bronchial epithelial cells (hBEpC), tracheal epithelial cells (hTEpC), dermal microvascular endothelial cells (HDMEC), keratinocytes, hepatocytes, renal proximal tubule epithelial cells (RPTEC), iPSC-derived astrocytes, cardiomyocytes, and GABA neurons (FIG. 18). All normal cells were obtained from HLA-A*02:01-positive donors (HLA-A*02:01 expression was confirmed by RNASeq). Importantly, as these normal cells can present highly diverse peptides on HLA-A*02:01, this serves as an assay system to assess a broad range of off-target effects. The B-CPAP cancer cell line with MAGE-B2 and HLA-A*02:01 expression was used as a positive control target cell. Mock (untransduced) T cells or T cells expressing an IL12-RFP construct (with no transgenic TCR) from the same donor were included as negative control effector cells. Production of cytokines (IFNγ, IL-12p70, TNFα) and granzyme B, as well as target cell cytotoxicity (measured by caspase 3/7 cleavage) was assessed in co-culture with TCR-T-TL12 cells (FIG. 18). All 4 TCR-T-IL12 cells induced cytokine production and target cell cytotoxicity when cocultured with the positive control B-CPAP cells (MAGE-B2+ HLA-A*02:01+). Importantly, TCR2-IL12 and TCR4-IL12 did not mediate the production of cytokines or enhance caspase 3/7 cleavage when co-cultured with any of the normal human primary or iPSC-derived cells tested, indicating no off-target reactivity against any of the normal cells tested.

### Assessment of Alloreactivity Potential Using 34 BLCL Lines

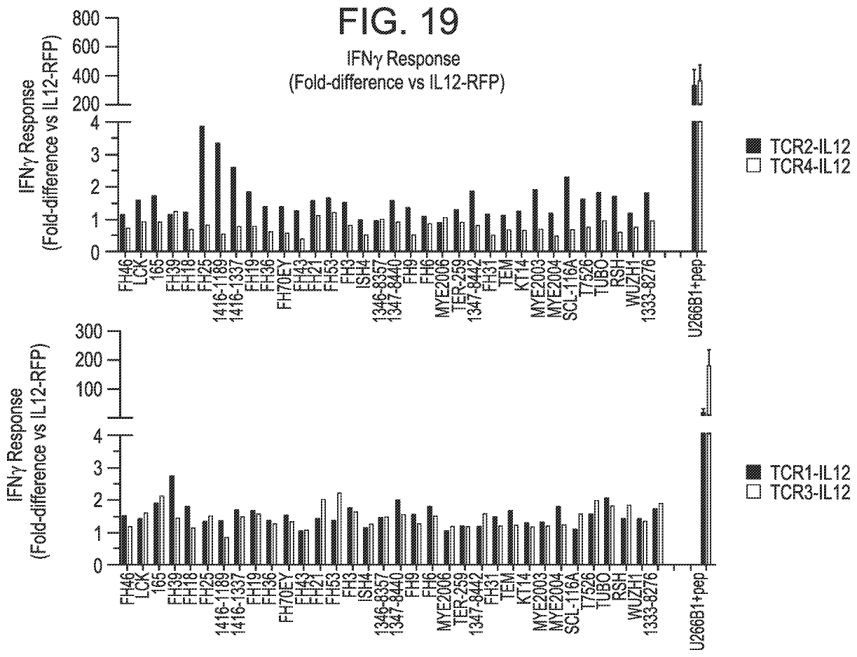

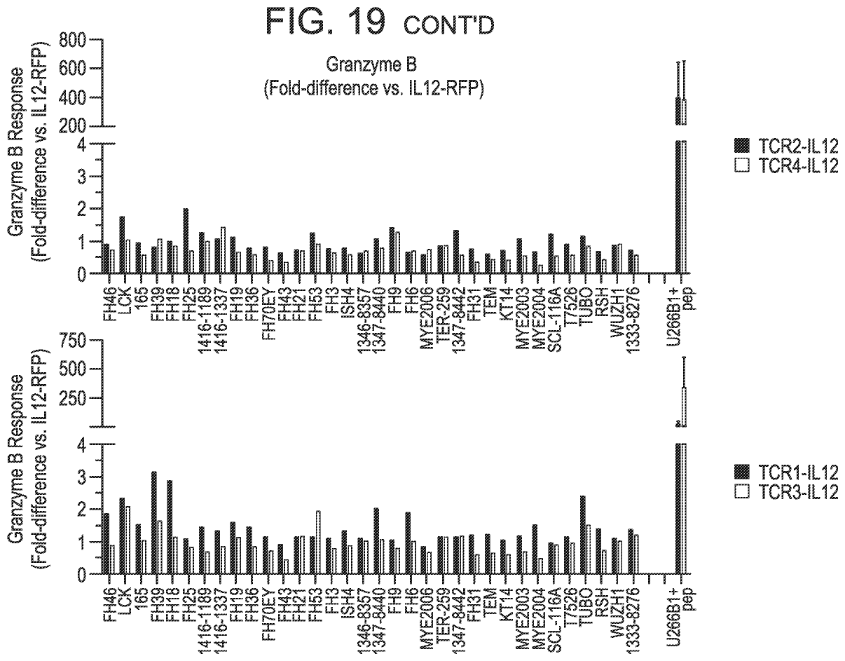

As a part of safety assessment, alloreactivity potential was evaluated by using a panel of 34 BLCLs (B lymphoblastoid cell lines) representing highly frequent (>11%) MHC Class I alleles in major US ethnic groups, including 38 HLA-A, 40 HLA-B, and 24 HLA-C alleles. Alloreactivity potential was evaluated by the production of cytokines (IFNγ, TNFα, and IL-12p70) and granzyme B when TCR-T-IL12 cells were co-cultured with each of the BLCLs. No significant increases in cytokine or granzyme B responses (greater than or equal to 4-fold compared to IL12-RFP control T cells) against the 34 BLCLs tested were observed for any of the four TCR-T-IL12 cells (FIG. 19). Some low-level responses (greater than or equal to 3-fold, but lower than 4-fold, compared to IL12-RFP control cells) were observed for TCRT-IL12 and TCR2-IL12. All four TCR-T-IL12 cells demonstrated robust cytokine and granzyme B responses against a positive control U266B1 cells (HLA-A*02:01+ MAGE-B2+ MAGE-A4+) pulsed with MAGE-B2 peptide.

Overall, the four exemplary TCR-T-IL12 candidates did not show significant safety concerns based on the normal and alloreactivity potential safety assessments performed.

### Methods and Materials Used in the Above Examples

MAGE-B2 pMHC-Specific TCR Identification by Healthy Donor Screen

### Generation of Autologous Antigen Presenting Cells (APCs)

Fresh or frozen HLA-A*02:01 positive healthy donor peripheral blood mononuclear cells (PBMCs) were used. Monocytes were positively selected by using human CD14-microbeads (Miltenyi Biotec, San Diego, CA, 130-050-201) from PBMCs. Mature dendritic cells were obtained by using CellXVivo™ Human Monocyte-derived Dendritic Cell (DC) Differentiation Kit (R&D, Minneapolis, MN, CDK004). Antigen presenting B cells were generated by using CD40L and IL-4 stimulation method. B cells were positively selected by using human CD19-microbeads (Miltenyi Biotec, 130-050-301) from PBMCs. CD19+ cells were then stimulated by 0.125 ug/ml recombinant huCD40L in B cell media and seeded in 24-well plate at 2×10⁵ cells/ml and 1 ml/well. B-cell media comprised of IMDM, GlutaMax™ supplement media (Gibco, 31980030) supplemented with 10% heat inactivated human serum (MilliporeSigma H3667-100ML), 100 U/ml penicillin and 100 ug/ml streptomycin (Gibco, 15140-122), 10 μg/ml gentamicin (Gibco, 15750-060) and 200 IU/ml IL-4 (Peprotech, Rock Hill, NJ, 20004100UG). Fresh B cell media with 400 IU/ml IL-4 was added to the B cell culture at 1 ml/well on day 3 post B cell activation without disturbing the cells. Activated B cells were ready to use for antigen-reactive T cell stimulation on day 6 post B cell activation.

### Ex Vivo Stimulation and Expansion of Antigen-Specific T Cells

MAGE-B2 peptide (Anaspec customized peptide, Freemont, CA) was added to the immature dendritic cells at 1p M along with recombinant human TNF-α on day 7 post CD14+ cell isolation. On day 9 post CD14+ cell isolation, MAGE-B2 peptide-pulsed mature dendritic cells were collected, washed, and mixed with CD14− PBMCs at ratio 1 to 10 in human T cell media with 10 μM MAGE-B2 peptide, 10 IU/ml IL-2 (Miltenyi Biotec, 130-097-745) and 10 ng/ml IL-7 (Peprotech, AF20007100UG). Human T cell complete media consists of a 1 to 1 mixture of CM and AIM-V™ (ThermoFisher, 12055083). CM consists of RPMI 1640 supplemented with GlutaMAX™ (Gibco, 61870-036, ThermoFisher), 10% human serum (MilliporeSigma, H3667), 25 mM HEPES (Gibco, 15630-080, ThermoFisher) and 10 μg/ml gentamicin (Gibco, 15750-060, ThermoFisher). MAGE-B2 specific T cells were further expanded by one to three rounds of weekly peptide-pulsed B cell activation (total up to four T cell antigen specific stimulations). HuCD40L activated B cells were collected, washed, and seeded in 6-well plate at 1×10⁶ cells/ml and 4 ml/well, 1 μM MAGE-B2 peptide was added to the B cells and incubated at 37° C. for 2 hours in the incubator. The peptide-pulsed B cells were then mixed with the T cells at a ratio of 1:10 in human T cell media with 10 IU/ml IL-2 and 10 ng/ml IL-7. MAGE-B2 dextramer positive cells were confirmed by flow cytometry and then sorted for TCR identification by single cell RNAseq.

### Sorting of Activated Antigen-Specific T Cells

MAGE-B2 peptide activated antigen-specific T cells were stained with MAGE-B2 dextramer-APC and -PE at room temperature in dark for 10 min and then stained by CD3-FITC (Biolegend, San Diego, CA, 300440) and CD8-BV605 (BD Biosciences, San Jose, CA, 564116). The dead cell exclusion stain (Sytox blue) was purchased from ThermoFisher (Invitrogen, S34857). Cells were sorted using an Aria™ Fusion cell sorter (BD Biosciences, San Jose, CA). Data were analyzed using Flowjo post-sort.

### Elispot

The sorted CD3+CD8+Dex+ T cells were validated for the antigen-specific IFNγ production by BD® ELISPOT assay (BD Bioscience, San Jose, CA, 551849) using peptide-loaded T2 cells. T2 cells were loaded with 10 μM MAGE-B2 peptide in human T cell complete media at 2×10⁶ cells/ml and 1 ml/well in 24 well plate for 1-2 hours. 150 ul of human T cell complete media and 50 μl of peptide-loaded T2 cells were added to each well in the pre-coated ELISPOT plate. The CD3+CD8+Dex+ T cells (500 or 1000 cells) were directly sorted into each well in the ELISPOPT plate. The ELISPOT was detected after 24-hour incubation in 37° C. incubator. The ELISPOT plates were scanned and counted by IMMUNOSPOT® (Cellular Technology Limited, Cleveland, OH).

### Single Cell RNAseq

Samples were processed using a Chromium™ Controller (10× Genomics, Pleasanton, CA) with the V(D)J single-cell Human T Cell enrichment kit (PN-1000006, PN-1000005, PN-120236, PN-120262) according to manufacturer's instructions for direct target enrichment, skipping cDNA amplification step for the full transcriptome. Briefly, cells and beads with barcoded oligonucleotides were encapsulated in nanoliter droplets where the cells were lysed, and mRNA reverse transcribed with poly-T primers and barcoded template-switch oligos. Nested PCR was then performed with primers in the constant region of the human TCR and template-switch oligo. The second target enrichment PCR was performed using 13-17 cycles depending on estimated cell input number according to manufacturer's suggestions. The final sequencing library was generated from fragmented PCR product ligated to Illumina sequencing adapters. Libraries were sequenced with 151 paired end reads (151×8×0×151) on NextSeq™ 550 or MiSeq™ (Illumina, Inc., San Diego, CA) at a depth of at least 5,000 reads per cell. Data was demultiplexed and analyzed with cellranger vdj (2.2.0) to obtain full-length paired TCR sequences assigned to individual cells.

Cloning and Transduction of TCRs into Jurkat Cells

Candidate TCRs were generated as gene fragments. Each fragment was cloned into a lentiviral expression vector consisting of a MSCV promoter and an IRES-driven eGFP for monitoring transfection or transduction. Successful transformants were screened by Sanger sequencing and verified clones were maxi-prepped for downstream applications. In those cases where transduction was used to screen a candidate TCR, the lentiviral vector was packaged into VSV-G pseudotyped virions (Alstem, Richmond, CA). Lentivirus carrying TCRs were transduced into a Jurkat TCR KO reporter cell line expressing CD8a constitutively and Renilla luciferase under a NFAT inducible promoter. Briefly, 20 μL of lentivirus particles were added to between 1000K and 1 million cells in complete media containing 5 ug/mL Polybrene (MilliporeSigma, TR1003G) in a 50 mL conical tube such that the multiplicity of infection (MOI) was 10. After the addition of virus, cells were spun at 1200×g for 45 min at 32° C. After the spin, the media was aspirated and replaced with sufficient fresh media to adjust the cells to a concentration of 500K cells/ml before being placed in a 37° C. incubator. Approximately 72 hours post-transduction, cells were analyzed by flow cytometry. 50 μl of cells were transferred to a 96-well U-bottom plate and 150 ul FACS buffer (PBS w/o CaCl₂) & MgCl₂ (Corning, Corning, NY, 21-040-CV)+5% FBS (Gibco, 10082-147)) added before being centrifuged at 300×g for 3 min. Supernatant was removed and cells were resuspended in 50 μl of 1× Fc block in FACS buffer which was incubated at 4° C. for 20 min. Fluorescent dextramer specific to MAGE-B2 peptide-MHC (GVYDGEEHSV/HLA-A*02:01, Immudex customized, Fairfax, VA) was incubated with transduced cells at room temperature for 10 min in the dark using the manufacturer's recommended concentration. Afterward, a 2× antibody cocktail containing anti-CD3 (BD Biosciences) in 50 ul volume was added before another incubation at 4° C. for 20 min. Cells were washed three times after staining by centrifugation at 300×g for 3 min followed by aspiration and resuspension. Prior to analysis, cells were fixed in 100 μl of fresh 2% formaldehyde solution at 4° C. for 20 min. Cells were washed twice to remove the formaldehyde before final suspension in 200 μl of PBS with EDTA. Fixed, labeled cells were run on either LSRII or Symphony™ cytometers (BD Biosciences) using recommended acquisition settings.

### Jurkat Activation Assay

Antigen-presenting T2 cells (ATCC) were loaded with peptides (Anaspec customized) or vehicle only at a range of concentrations in serum-free media for two hours. After incubation, loaded T2 cells were washed three times before being resuspended in complete media, counted and seeded at 15,000 cells/well in a half area 96 well plate (Corning). Successfully transduced Jurkat cells were added at 30,000 cells/well to a total volume of 100 μL. The TCR-expressing Jurkat cells were co-cultured at 37° C. in the presence of the T2 cells for 24 hours. At the end of this incubation, the plate was briefly centrifuged at 300×g before half the volume was harvested and stored for characterization of cytokine secretion. To the remaining volume was added an equal volume of RENILLAGLO® (Promega) and the plate was incubated for 20 min at room temperature with shaking before luminescence was detected on an ENVISION® (Perkin Elmer, Waltham, MA). The activities of individual TCRs were expressed as the fold change of the luminescence in the presence of T2 cells loaded with peptide compared to co-cultures with vehicle-only T2 cells.

### MAGE-B2 TCR-T and TCR-T-IL12 Cell Production Using Human Primary T Cells

PBMCs from three healthy donors (HLA-A*02:01) were isolated from leukopak (Allcells, Alameda, CA) using Ficoll-Paque gradient centrifugation, with additional T cell isolation by using CD3 negative selection kit (Miltenyi Biotec, 130-096-535) and associated manufacturer's protocol. One day before TCR transduction, frozen pan-T cells were thawed and resuspended in Human T cell complete media at 1×10⁶ cells/ml, and were stimulated by CD3/CD28 Dynabeads™ (Thermo Fisher, 11131D) with T cells to beads ratio (2:1) in the presence of 30 IU/ml IL-2 (Miltenyi Biotec, 130-097-745), 10 ng/ml IL-7 (Peprotech, AF20007100UG) and 25 ng/ml IL-15 (Peprotech, AF20015100UG). The T cells were then seeded at 1 ml per well in 24-well plates. On the day of TCR transduction, activated T cells (300K) were seeded in Human T cell complete media per well in 48-well plate and transduced with lentivirus in the presence of 8 μg/ml polybrene, 100 IU/ml IL-2, 10 ng/ml IL-7 and 25 ng/ml IL-15. The T cells were then spin-inoculated at 1500×g for 1.5 hours at 32° C. After spin-inoculation, 380 ul of media with 8 μg/ml polybrene, 100 IU/ml IL-2, 10 ng/ml IL-7, and 25 ng/ml IL-15 was added to the cells to make a total volume of 600 μl per well. At 17-18 hours post transduction, ˜400 μl of media was removed without touching the cells at the bottom of the wells. The cells from each well of 48-well plate were transferred to one well of G-REX® 24-well plate (WilsonWolf, St Paul, MN, P/N 80192M) in 3 ml of Human T cell complete media containing 100 IU/ml IL-2, 10 ng/ml IL-7 and 25 ng/ml IL-15. On day 4 post transduction, the dynabeads were removed according to manufacturer's protocol. The TCR-T cells were seeded to G-REX® 6-well plate (WilsonWolf, P/N 80240M) at ˜10×10⁶ cells in 30 ml media per well in the presence of 100 IU/ml IL-2, 10 ng/ml IL-7, and 25 ng/ml IL-15. On day 7 post transduction, the TCR-Ts were harvested, frozen down and stored in liquid nitrogen vapor phase. TCR transduction efficiency was validated by dextramer binding. The TCR-T-IL12 cells were produced by the process described in the patent application (PCT published application number: WO 2021/211104).

### Flow Cytometry

The following antibodies were used for T cell phenotyping: CD3-FITC (Biolengend: 300440), CD8-BV605 (BD: 564116), CD4-PE (Biolegend: 317410). The following antibodies were used for dendritic cell phenotyping: CD14-PerCP/Cy5.5 (Biolegend: 301824), CD11c-PE (Biolegend: 337206), CD1a-APC-cy7 (Biolegend: 300125), CD86-APC (BD: 555660). The following antibodies were used for B cell phenotyping: MHC class I (Biolegend: 311414), MHC class II (Biolengend: 361706), CD83-PE (BD 556855), CD86-APC (BD: 555660), CD20-FITC (BD: 556632). Dextramers-APC or -PE were purchased from Immudex (customized dextramers). 50 nM PKI dasatinib (Axon Medchem: 1392) was used to prevent TCR internalization. The TCR expressing T cells were incubated with 50 nM PKI dasatinib at 37° C. for 30 min and then followed by dextramer staining on ice for 30 min and cell surface marker staining at 4° C. for 15 min. The dead cell exclusion stain (Sytox blue, ThermoFisher/Invitrogen, 534857) was used. Flow cytometry data were analyzed using Flowjo.

### T Cell-Mediated T2-Luc/Peptide Cytotoxicity Assay (T2/Peptide TDCC Assay)

Functionality and killing specificity of MAGE-B2 TCR-T was determined by T2-luc (T2 cell line expressing firefly luciferase) killing assays. T2-Luc cells were collected, washed and resuspended at 2×10⁶ cells/ml in T2-Luc killing assay media (RPMI 1640-GlutaMAX™, 1× Non-Essential Amino Acids Solution (Gibco, 11140-050, ThermoFisher, Waltham, MA), 10 mM HEPES (Gibco, 15630-080), 50 μM 2-β-mercaptoethanol (Gibco, 21985-023), 1 mM sodium pyruvate (Gibco, 11360-070), 100 U/ml Penicillin-Streptomycin (Gibco, 15140-122), 5% heat-inactivated FBS (Gibco, 10082-147), and then seeded at 1 ml per well in 24-well plate. T2-Luc cells were pulsed with the indicated peptide concentrations for two to four hours at 37° C. T2-Luc cells were then washed and resuspended at 1×10⁵ cells/ml and were seeded at 25 μl per well in 384-well plates (Corning, 3570). T2-Luc cells were incubated with 25 μl of TCR-T cells with the indicated dextramer+ TCR-T to T2-luc cells ratio for 48 hours. The luminescent signal was measured by addition of 30 μl of Bio-Glo™ (Promega, Madison, WI, G7940) followed by measurement of luminescent signals by using Biostack™ neo system (BioTek, Winooski, VT). For parental TCR-T, prior to the killing assays, all of the TCR-T-IL12 cells were not normalized by adding mock T cells. different TCR-Ts were normalized to the same amount of MAGE-B2 dextramer+ cells (e.g. 10%) by adding mock (untransduced) T cells. Specific lysis (specific killing %) was calculated through normalization of TCR-T+T2/target peptide killing either by mock T cells+T2/target peptide killing or by TCR-T+T2/no peptide killing. Specific lysis formulas are described below.

Formula for Specific Lysis (%)

Peptide Titration (MAGE-B2/A4 Peptides and Similar Peptides):

{1−(TCRT+T2-luc/test peptide RLU)/(TCRT+T2-luc/no peptide RLU)}×100

E:T Titration (MAGE-B2/A4 Peptides):

{1−(TCRT+T2-luc/MAGE-B2 peptide RLU)/(MockT+T2-luc/MAGE-B2 peptide RLU)}×100

Cancer Cell Line Killing:

{1−(TCRT+cancer cell line RLU)/(MockT+cancer cell line RLU)}×100

### T Cell-Mediated Cancer Cell Cytotoxicity Assay (Cancer Cell TCDD Assay)

Cytotoxicity of TCR-T cells against MAGE-B2 positive and negative cancer cell lines was determined by cancer cell killing assay. Cancer cells were collected, washed and resuspended at 1×10⁵ cells/ml in cancer cell killing assay media (RPMI 1640-GlutaMAX™, 1× Non-Essential Amino Acids Solution (Gibco, 11140-050, ThermoFisher), 10 mM HEPES (Gibco, 15630-080, ThermoFisher), 50 μM 2-β-mercaptoethanol (Gibco, 21985-023, ThermoFisher), 1 mM sodium pyruvate (Gibco, 11360-070, ThermoFisher), 100 U/ml Penicillin-Streptomycin (Gibco, 15140-122, ThermoFisher), 10% heat-inactivated FBS (Gibco, 10082-147, ThermoFisher). Cancer cells were then seeded at 25 μl per well in 384-well plates and incubated with 25 μl of TCR-T cells with the indicated dextramer+ TCR-T to T2-Luc cells ratio for 48 hours. Following incubation, for adherent cancer cells, the suspension T cells were removed, and wells were washed with DPBS with Ca²⁺Mg²⁺ (Corning, 21-031-CM) using a plate washer. The luminescent signal was measured by addition of 30 μl of Celltiter Glo (Promega, G7573). For suspension luciferase labeled cancer cells, the luminescent signal was measured by the addition of 30 μl of Bio-Glom (Promega, G7940). Biostackm neo system was used for luminescence measurement. For suspension cancer cells without luciferase labeling, cancer cells were labeled by Celltrace far red (Invitrogen, C34572, Carlsbad, CA, USA). Cancer cells were resuspended in serum free RPMI media containing Celltracem far red (1:4000 dilution) at 1×10⁶ cells/ml and were incubated at 37° C. for 10 min. The reaction was stopped by adding 30 ml killing assay media and incubating at room temperature for 10 min. Live cancer cells were detected by flow cytometry. The dead cell exclusion stain (Sytox™ blue, ThermoFisher/Invitrogen, S34857) was used. Specific lysis (specific killing %) was calculated through normalization of TCR-T killing against a cancer cell line by mock T cell killing or IL 12-RFP T cell killing against a cancer cell line. Specific lysis formula is described above.

### Similar Peptide Screen

Functional specificity of MAGE-B2 TCR-T was determined using T2-Luc/peptide directed killing assays. Peptides including target and similar peptides were synthesized by JPT (Berlin, Germany) or AnaSpec (Fremont, CA). T2-Luc cells were incubated with reactive similar peptides, target specific peptide or DMSO control in T2-Luc killing media at a final peptide concentration range of 1.0E-05M to 6.0E-16M (potency) or 1.0E-05M (single point) for 2 hours at 37° C./5% CO₂. Frozen MAGE-B2 TCR-T and mock T cells were thawed, washed, and rested in human T cell media for 3 hrs prior to assay set-up. MAGE-B2 TCR-T cells were washed 3× in assay media and re-suspended at 2.5E06 cells/mL. Peptide loaded T2-Luc cells were added to white-clear bottom 384-well assay plates (Costar) at 2,000 cells/25 μL using Bravo liquid handling system (Agilent, Santa Clara, CA). MAGE-B2 TCR-T cells were prepared by diluting MAGE-B2 dextramer positive cells with mock T-cells to obtain a 10:1 target: effector ratio; 20,000 cells/25 μL (final 1:1 Dex+ T cell: T2-Luc). T2-Luc pulsed cells and TCR-T cells were incubated for 48 hours at 37° C./5% CO2. T2-Luc cell viability was determined using Bio-Glo™ Luciferase Assay System (Promega, G7940) according to the manufacturer's recommendation. Luminescence was detected using ENVISION® Multilabel Plate Reader (Perkin Elmer, Santa Clara, CA). Percent viability was calculated using the following formula: % Viability=(Sample raw RLU value/Average DMSO control RLU)×100. EC50 was determined using GraphPad Prism (non-linear regression curve fit analysis).

### Human Primary Normal Cell Culture

Sources of human primary normal cells and iPSC-derived cells are summarized in Table 4. Culture conditions for those cells are summarized in Table 5. Primary cells were thawed and cultured according to the supplier's instructions with the following exceptions: cardiomyocytes, astrocytes, GABA neurons, and RPTEC which were converted into RPMI 1640 culture medium just prior to the initiation of coculture. Prior optimization studies demonstrated a tolerability of RPMI 1640 and improvement in cell viability for these cell types. All cells were counted and assessed for viability prior to assay.

TABLE 4
Source of human normal primary and iPSC-derived cells

| Cells | Cell Type | Source | Donor | Catalog # |
|---|---|---|---|---|
| Bronchial | Primary | PromoCell, | 424Z015.3 | C-12640 |
| Epithelial | | Heidelberg, | | |
| Cells (hBEpC) | | Germany | | |
| Renal Proximal | Primary | Lonza, Basel, | 617045 | CC-2553 |
| Tubule Epithelial | | Switzerland | | |
| Cells (RPTEC) | | | | |
| Tracheal | Primary | PromoCell | 446Z036.8 | C-12212 |
| Epithelial | | | | |
| Cells (hTEpC) | | | | |
| Keratinocytes | Primary | PromoCell | 425Z026.2 | C-12003 |
| Dermal | Primary | PromoCell | 435Z034.2 | C-12212 |
| Microvascular | | | | |
| Endothelial | | | | |
| Cells (HDMEC) | | | | |
| Hepatocytes | Primary | Lonza | HUM17299A, | HUCPG |
| | | | HUM173531 | |
| GABA Neurons | iPSC | Cellular | 01434 | R1013 |
| | | Dynamics, | | |
| | | Madison, WI | | |
| Astrocytes | iPSC | Cellular | 01434 | R1092 |
| | | Dynamics | | |
| Cardiomyocytes | iPSC | Cellular | 01434 | R1007 |
| | | Dynamics | | |
| B-CPAP | Thyroid carcinoma | DSMZ, | N/A | N/A |
| | cell line | Braunschweig, | | |
| | (MAGE-B2⁺) | Germany | | |

TABLE 5
Culture media and methods for human normal cells

| | | | | Plating |
|---|---|---|---|---|
| | Assay | | Specific | Density |
| Cells | Medium | Supplements | Methods | (Cells/Well) |
| Bronchial | Airway | Required | Plated cells directly | 20,000 |
| Epithelial | Epithelial | supplements | into 96-well | |
| Cells | Cell | contained in kit | ViewPlates | |
| (hBEpC) | Medium | (hydrocortisone | | |
| | | omitted) | | |
| Renal | RPMI with | 10% HI FBS, | Thawed and | 20,000 |
| Proximal | supplements | Pen/Strep | maintained cells in | |
| Tubule | | | REGM. Plated cells | |
| Epithelial | | | directly into 96-well | |
| Cells | | | ViewPlates | |
| (RPTEC) | | | | |
| Tracheal | Airway | Required | Plated cells directly | 20,000 |
| Epithelial | Epithelial | supplements | into 96-well | |
| Cells | Cell | contained in kit | ViewPlates | |
| (hTEpC) | Medium | (hydrocortisone | | |
| | | omitted) | | |
| Keratinocytes | Keratinocyte | Required | Plated cells directly | 20,000 |
| | Growth | supplements | into 96-well | |
| | Medium | contained in kit | ViewPlates | |
| | | (hydrocortisone | | |
| | | omitted) | | |
| Dermal | Endothelial | Required | Plated cells directly | 20,000 |
| Microvascular | Cell Growth | supplements | into 96-well | |
| Endothelial | Medium | contained in kit | ViewPlates | |
| Cells | | (hydrocortisone | | |
| (HDMEC) | | omitted) | | |
| Hepatocytes | Hepatocyte | Required | Thawed in | 30,000 |
| | Maintenance | supplements | Hepatocyte Thaw | |
| | Medium | contained in kit | Medium; plated in | |
| | | (hydrocortisone | William's Medium E | |
| | | omitted) | with Hepatocyte | |
| | | | Plating Supplements | |
| | | | into collagen-coated | |
| | | | 96-well ViewPlates; | |
| | | | after 24 hr incubation, | |
| | | | cells washed and | |
| | | | assayed in Hepatocyte | |
| | | | Maintenance Medium | |
| GABA Neurons | RPMI with | 10% HI FBS, | Plated directly in | 20,000 |
| | supplements | Pen/Strep | iCell Neural Base | |
| | | | Medium with Neural | |
| | | | Supplement A into | |
| | | | 96-well PDL-coated | |
| | | | ViewPlates coated | |
| | | | with 3.33 ug/mL | |
| | | | Laminin. After 24 hr | |
| | | | incubation, cells were | |
| | | | washed and assayed | |
| | | | in RPMI | |
| Astrocytes | RPMI with | 10% HI FBS, | Plated directly in | 20,000 |
| | supplements | Pen/Strep | DMEM with N-2 | |
| | | | Supplement A into | |
| | | | 96-well ViewPlates. | |
| | | | After 24 hr | |
| | | | incubation, cells were | |
| | | | washed and assayed | |
| | | | in RPMI | |
| Cardiomyocytes | RPMI with | 10% HI FBS, | Plated directly in | 20,000 |
| | supplements | Pen/Strep | iCell Cardiomyocyte | |
| | | | Plating media into 96- | |
| | | | well ViewPlates | |
| | | | coated with 0.1% | |
| | | | gelatin. After 24 hr | |
| | | | incubation, cells were | |
| | | | washed with iCell | |
| | | | Cardiomyocyte | |
| | | | Maintenance | |
| | | | Medium. Media | |
| | | | replaced every other | |
| | | | day until spontaneous | |
| | | | beating is observed. | |
| | | | Cells were washed | |
| | | | again in Maintenance | |
| | | | Media and assayed in | |
| | | | RPMI. | |
| B-CPAP | RPMI with | 10% HI FBS, | Plated cells directly | 20,000 |
| | supplements | Pen/Strep | into 96-well | |
| | | | ViewPlates | |

Cytotoxicity Assays with Human Primary Normal Cells

Target cell cytotoxicity was assessed using a phase contrast/fluorescence kinetic imaging assay. Fluorescent caspase 3/7 cleavage was measured over time with an INCUCYTE® live imaging device (Sartorium, Gottingen, Germany) and overlaid onto phase contrast images that captured cell confluence. Prior to implementing the cytotoxicity assay, different plating densities and tolerability to various culture media were assessed to achieve suitable confluence without significant cell overlap in 96-well plates. Target cells (100 μl) were added at the densities listed in Table 3 to black 96-well ViewPlates containing 50 μl of MAGE-B2 TCR-T-IL12 cells, IL-12 RFP T cells, or mock T cells at a dextramer-normalized effector: target (E:T) ratio of 1:1, by taking into consideration the dextramer positivity of each TCR-T construct. CellEvents caspase 3/7 reagent (50 μl) was added according to the manufacturer's instructions (ThermoFisher, C10423). Assay plates were placed in a 37° C., 5% CO₂ incubator equipped with an INCUCYTE© S3. Phase contrast and fluorescent images (5 fields) with the 10× objective were collected every 4 hours starting at 0 hour for 44 or 48 hours and analyzed for Caspase 3/7 total integrated intensity using INCUCYTE® 2019B software. After 44 or 48 hours, plates were removed from the incubator and 50 μL of cell culture medium was removed from the wells for cytokine analysis.

Cytokine Assay with Human Primary Normal Cells

Cell culture supernatants (50 μL) were collected from cytotoxicity assays at 44 or 48 hours into 96-well plates. Plates were sealed and stored at −80° C. for cytokine analysis on subsequent days. Supernatants were thawed according to manufacturer's instructions. IFNγ and IL-12p70 plates were blocked with blocking buffer from the MSD kit (1% w/v in PBS) for 1 hour at room temperature with shaking. After washing the plates three times with PBS/0.05% Tween-20, calibrators and samples (25 μL undiluted) were added according to plate layouts. Detection antibody was added (25 μL) and plates were incubated at room temperature for 2 hours with shaking, followed by 3 washes with PBS/0.05% Tween-20. Read Buffer (2×, 150 μL) was added to each well and plates were analyzed on the MSD MESOSECTOR® S600 instrument (Meso Scale Diagnostics, Rockville, MD). Standard curves were generated from calibrators and used to quantitate cytokines in samples using MSD DISCOVERY WORKBENCH® software 4.0.

### Alloreactivity Screen

Alloreactivity potential was assessed by co-culturing each of the 4 TCR-T-IL12 cells with each of 34 BLCL lines (B lymphoblastoid cell lines) representing 39 HLA-A, 40 HLA-B and 23 HLA-C alleles. BLCLs were purchased from Fred Hutchinson Cancer Research Institute (Seattle, WA) and Cellero (Bothell, WA) as listed in Table 6. BLCLs were cultured in 15% FBS complete RPMI containing: RPMI-1640 with L-Glutamine, 15% (v/v) HI-FBS, and 1 mM Sodium Pyruvate.

U266B1 cells (ATCC; 10⁵ cells/ml in media) as a MAGE-B2+MAGE-A4+HLA-A*02:01+positive control cell line were pulsed with 50 μM MAGE-B2 peptide by incubation at 37° C. for 2 hours. TCR-T cells from donor D160780 were thawed by addition of media, centrifuged at 400×g for 5 min at 4° C., resuspended in 10 ml of media and counted. 1.923×10⁵ TCR-T cells were co-cultured with either 1×10⁴ BLCLs or peptide-pulsed U266B1 cells in 200 μl volume. The dextramer-normalized effector:target ratios for the 4 TCR-T cells ranged from 3:1 to ˜8:1, depending upon the respective dextramer-positivity. All co-cultures were conducted in 96-well flat-bottom tissue culture plates at 37° C., 5% CO2 for 48 hours. Following incubation, the 96-well plates were centrifuged at 887×g for 1 min at 4° C. and the supernatant was collected into 96-well V-bottom plates for cytokine analysis. Cytokines and Granzyme B were evaluated by LUMINEX® assay using a custom MILLIPLEX© Human Cytokine/Chemokine Kit (Millipore, ST Louis, MO, SRP1885), including the analytes of IFNγ, granzyme B, TNFα and IL-12p70, as per manufacturer instructions. Serial dilutions of analyte standards were run in replicates on each assay plate. The LUMINEX® plate was read on a FLEXMAP 3D® instrument (XMAP® technologies, Luminex). Data was exported by XPONENT® Software (Luminex), and analyzed directly by EMD Millipore's MILLIPLEX® Analyst software (Burlington, MA), generating standard curves using a 5-parameter logistic non-linear regression fitting curve. The limits of detection (Min and Max) were calculated by the MILLIPLEX® Analyst software (Millipore) as the result of the average of appropriate replicate standard curve values obtained from each assay plate and indicate the range within which an analyte can be interpolated from the standards. Samples were run at appropriate dilutions to ensure measurements of sample analyte levels were within assay standard curve limits. Cytokine and granzyme B levels are reported in pg/mL or as fold-differences over IL12 T cells (control) and graphed in GraphPad Prism software (GraphPad, San Diego, CA).

TABLE 6
BLCLs for alloreactivity screen

| Cell Line Name | IHW Reference | Vendor |
|---|---|---|
| 1346-8357 | IHW01080 | Fred Hutch |
| 1347-8440 | IHW01103 | Fred Hutch |
| 1347-8442 | IHW01105 | Fred Hutch |
| 1416-1189 | IHW01176 | Fred Hutch |
| 1416-1337 | IHW01185 | Fred Hutch |
| FH19 | IHW09400 | Fred Hutch |
| FH31 | IHW09413 | Fred Hutch |
| FH39 | IHW09427 | Fred Hutch |
| FH46 | IHW09434 | Fred Hutch |
| FH70EY | IHW09458 | Fred Hutch |
| LCK | IHW09367 | Fred Hutch |
| TEM | IHW09057 | Fred Hutch |
| 165 | — | Cellero |
| FH18 | IHW09398 | Fred Hutch |
| FH21 | IHW09403 | Fred Hutch |
| FH25 | IHW09407 | Fred Hutch |
| FH3 | IHW09375 | Fred Hutch |
| FH36 | IHW09423 | Fred Hutch |
| FH43 | IHW09431 | Fred Hutch |
| FH53 | IHW09441 | Fred Hutch |
| FH6 | IHW09380 | Fred Hutch |
| FH9 | IHW09383 | Fred Hutch |
| ISH4 | IHW09371 | Fred Hutch |
| KT14 | IHW09103 | Fred Hutch |
| MYE 2003 | IHW09419 | Fred Hutch |
| MYE 2004 | IHW09420 | Fred Hutch |
| MYE 2006 | IHW09422 | Fred Hutch |
| SCL-116A | IHW09465 | Fred Hutch |
| T7526 | IHW09076 | Fred Hutch |
| TER-259 | IHW09401 | Fred Hutch |
| TUBO | IHW09045 | Fred Hutch |
| RSH | IHW09021 | Fred Hutch |
| WUZHI | IHW09459 | Fred Hutch |
| 1333-8276 | IHW01040 | Fred Hutch |

What is claimed is:

1. An expression vector comprising a nucleic acid sequence encoding a T-cell receptor (TCR) alpha chain and a TCR beta chain, wherein the TCR alpha chain and TCR beta chain are selected from the group consisting of:
a. a TCR alpha chain comprising an amino acid sequence set forth in SEQ ID NO:13 and a TCR beta chain comprising an amino acid sequence set forth in SEQ ID NO:24;
b. a TCR alpha chain comprising an amino acid sequence set forth in SEQ ID NO:14 and a TCR beta chain comprising an amino acid sequence set forth in SEQ ID NO:25;
c. a TCR alpha chain comprising an amino acid sequence set forth in SEQ ID NO:15 and a TCR beta chain comprising an amino acid sequence set forth in SEQ ID NO:26;
d. a TCR alpha chain comprising an amino acid sequence set forth in SEQ ID NO:16 and a TCR beta chain comprising an amino acid sequence set forth in SEQ ID NO:27;
e. a TCR alpha chain comprising an amino acid sequence set forth in SEQ ID NO:17 and a TCR beta chain comprising an amino acid sequence set forth in SEQ ID NO:28;
f. a TCR alpha chain comprising an amino acid sequence set forth in SEQ ID NO: 18 and a TCR beta chain comprising an amino acid sequence set forth in SEQ ID NO:29;
g. a TCR alpha chain comprising an amino acid sequence set forth in SEQ ID NO:19 and a TCR beta chain comprising an amino acid sequence set forth in SEQ ID NO:30;
h. a TCR alpha chain comprising an amino acid sequence set forth in SEQ ID NO:20 and a TCR beta chain comprising an amino acid sequence set forth in SEQ ID NO:31;
i. a TCR alpha chain comprising an amino acid sequence set forth in SEQ ID NO:21 and a TCR beta chain comprising an amino acid sequence set forth in SEQ ID NO:32;
j. a TCR alpha chain comprising an amino acid sequence set forth in SEQ ID NO:22 and a TCR beta chain comprising an amino acid sequence set forth in SEQ ID NO:33; and
k. a TCR alpha chain comprising an amino acid sequence set forth in SEQ ID NO:23 and a TCR beta chain comprising an amino acid sequence set forth in SEQ ID NO:34.

2. The expression vector of claim 1, further comprising a nucleic acid encoding interleukin-12 (IL-12) or a functional variant thereof.

3. The expression vector of claim 1, wherein the expression vector is a viral vector.

4. The expression vector of claim 3, wherein the viral vector is a retroviral vector.

5. The expression vector of claim 4, wherein the retroviral vector is a lentiviral vector.

6. The expression vector of claim 1, wherein the TCR alpha chain comprises an amino acid sequence set forth in SEQ ID NO:13 and the TCR beta chain comprises an amino acid sequence set forth in SEQ ID NO:24.

7. The expression vector of claim 6, wherein the TCR alpha chain comprises an amino acid sequence set forth in SEQ ID NO:35 and the TCR beta chain comprises an amino acid sequence set forth in SEQ ID NO 46.

8. The expression vector of claim 7, wherein the TCR alpha chain comprises an amino acid sequence set forth in SEQ ID NO:57 and the TCR beta chain comprises an amino acid sequence set forth in SEQ ID NO:68.

9. The expression vector of claim 1, wherein the TCR alpha chain comprises an amino acid sequence set forth in SEQ ID NO:14 and the TCR beta chain comprises an amino acid sequence set forth in SEQ ID NO:25.

10. The expression vector of claim 9, wherein the TCR alpha chain comprises an amino acid sequence set forth in SEQ ID NO:36 and the TCR beta chain comprises an amino acid sequence set forth in SEQ ID NO:47.

11. The expression vector of claim 10, wherein the TCR alpha chain comprises an amino acid sequence set forth in SEQ ID NO:58 and the TCR beta chain comprises an amino acid sequence set forth in SEQ ID NO: 69.

12. The expression vector of claim 1, wherein the TCR alpha chain comprises an amino acid sequence set forth in SEQ ID NO:15 and the TCR beta chain comprises an amino acid sequence set forth in SEQ ID NO:26.

13. The expression vector of claim 12, wherein the TCR alpha chain comprises an amino acid sequence set forth in SEQ ID NO:37 and the TCR beta chain comprises an amino acid sequence set forth in SEQ ID NO:48.

14. The expression vector of claim 13, wherein the TCR alpha chain comprises an amino acid sequence set forth in SEQ ID NO:59 and the TCR beta chain comprises an amino acid sequence set forth in SEQ ID NO:70.

15. The expression vector of claim 1, wherein the TCR alpha chain comprises an amino acid sequence set forth in SEQ ID NO:16 and the TCR beta chain comprises an amino acid sequence set forth in SEQ ID NO:27.

16. The expression vector of claim 15, wherein the TCR alpha chain comprises an amino acid sequence set forth in SEQ ID NO:38 and the TCR beta chain comprises an amino acid sequence set forth in SEQ ID NO:49.

17. The expression vector of claim 16, wherein the TCR alpha chain comprises an amino acid sequence set forth in SEQ ID NO:60 and the TCR beta chain comprises an amino acid sequence set forth in SEQ ID NO:71.

18. The expression vector of claim 1, wherein the TCR alpha chain comprises an amino acid sequence set forth in SEQ ID NO:17 and the TCR beta chain comprises an amino acid sequence set forth in SEQ ID NO:28.

19. The expression vector of claim 18, wherein the TCR alpha chain comprises an amino acid sequence set forth in SEQ ID NO:39 and the TCR beta chain comprises an amino acid sequence set forth in SEQ ID NO: 50.

20. The expression vector of claim 19, wherein the TCR alpha chain comprises an amino acid sequence set forth in SEQ ID NO:61 and the TCR beta chain comprises an amino acid sequence set forth in SEQ ID NO:72.

21. The expression vector of claim 1, wherein the TCR alpha chain comprises an amino acid sequence set forth in SEQ ID NO:18 and the TCR beta chain comprises an amino acid sequence set forth in SEQ ID NO:29.

22. The expression vector of claim 21, wherein the TCR alpha chain comprises an amino acid sequence set forth in SEQ ID NO:40 and the TCR beta chain comprises an amino acid sequence set forth in SEQ ID NO:51.

23. The expression vector of claim 22, wherein the TCR alpha chain comprises an amino acid sequence set forth in SEQ ID NO:62 and the TCR beta chain comprises an amino acid sequence set forth in SEQ ID NO:73.

24. The expression vector of claim 1, wherein the TCR alpha chain comprises an amino acid sequence set forth in SEQ ID NO:19 and the TCR beta chain comprises an amino acid sequence set forth in SEQ ID NO:30.

25. The expression vector of claim 24, wherein the TCR alpha chain comprises an amino acid sequence set forth in SEQ ID NO:41 and the TCR beta chain comprises an amino acid sequence set forth in SEQ ID NO: 52.

26. The expression vector of claim 25, wherein the TCR alpha chain comprises an amino acid sequence set forth in SEQ ID NO:63 and the TCR beta chain comprises an amino acid sequence set forth in SEQ ID NO:74.

27. The expression vector of claim 1, wherein the TCR alpha chain comprises an amino acid sequence set forth in SEQ ID NO:20 and the TCR beta chain comprises an amino acid sequence set forth in SEQ ID NO:31.

28. The expression vector of claim 27, wherein the TCR alpha chain comprises an amino acid sequence set forth in SEQ ID NO:42 and the TCR beta chain comprises an amino acid sequence set forth in SEQ ID NO:53.

29. The expression vector of claim 28, wherein the TCR alpha chain comprises an amino acid sequence set forth in SEQ ID NO:64 and the TCR beta chain comprises an amino acid sequence set forth in SEQ ID NO:75.

30. The expression vector of claim 1, wherein the TCR alpha chain comprises an amino acid sequence set forth in SEQ ID NO:21 and the TCR beta chain comprises an amino acid sequence set forth in SEQ ID NO:32.

31. The expression vector of claim 30, wherein the TCR alpha chain comprises an amino acid sequence set forth in SEQ ID NO:43 and the TCR beta chain comprises an amino acid sequence set forth in SEQ ID NO:54.

32. The expression vector of claim 31, wherein the TCR alpha chain comprises an amino acid sequence set forth in SEQ ID NO:65 and the TCR beta chain comprises an amino acid sequence set forth in SEQ ID NO:76.

33. The expression vector of claim 1, wherein the TCR alpha chain comprises an amino acid sequence set forth in SEQ ID NO:22 and the TCR beta chain comprises an amino acid sequence set forth in SEQ ID NO:33.

34. The expression vector of claim 33, wherein the TCR alpha chain comprises an amino acid sequence set forth in SEQ ID NO:44 and the TCR beta chain comprises an amino acid sequence set forth in SEQ ID NO:55.

35. The expression vector of claim 34, wherein the TCR alpha chain comprises an amino acid sequence set forth in SEQ ID NO:66 and the TCR beta chain comprises an amino acid sequence set forth in SEQ ID NO: 77.

36. The expression vector of claim 1, wherein the TCR alpha chain comprises an amino acid sequence set forth in SEQ ID NO:23 and the TCR beta chain comprises an amino acid sequence set forth in SEQ ID NO:34.

37. The expression vector of claim 36, wherein the TCR alpha chain comprises an amino acid sequence set forth in SEQ ID NO:45 and the TCR beta chain comprises an amino acid sequence set forth in SEQ ID NO:56.

38. The expression vector of claim 37, wherein the TCR alpha chain comprises an amino acid sequence set forth in SEQ ID NO:67 and the TCR beta chain comprises an amino acid sequence set forth in SEQ ID NO:78.

39. A cell expressing a recombinant T-cell receptor (TCR), said TCR comprising:
a. a TCR alpha chain comprising an amino acid sequence set forth in SEQ ID NO:13 and a TCR beta chain comprising an amino acid sequence set forth in SEQ ID NO:24;
b. a TCR alpha chain comprising an amino acid sequence set forth in SEQ ID NO:14 and a TCR beta chain comprising an amino acid sequence set forth in SEQ ID NO:25;
c. a TCR alpha chain comprising an amino acid sequence set forth in SEQ ID NO:15 and a TCR beta chain comprising an amino acid sequence set forth in SEQ ID NO:26;
d. a TCR alpha chain comprising an amino acid sequence set forth in SEQ ID NO:16 and a TCR beta chain comprising an amino acid sequence set forth in SEQ ID NO:27;
e. a TCR alpha chain comprising an amino acid sequence set forth in SEQ ID NO:17 and a TCR beta chain comprising an amino acid sequence set forth in SEQ ID NO:28;
f. a TCR alpha chain comprising an amino acid sequence set forth in SEQ ID NO:18 and a TCR beta chain comprising an amino acid sequence set forth in SEQ ID NO:29;
g. a TCR alpha chain comprising an amino acid sequence set forth in SEQ ID NO:19 and a TCR beta chain comprising an amino acid sequence set forth in SEQ ID NO:30;
h. a TCR alpha chain comprising an amino acid sequence set forth in SEQ ID NO:20 and a TCR beta chain comprising an amino acid sequence set forth in SEQ ID NO:31;
i. a TCR alpha chain comprising an amino acid sequence set forth in SEQ ID NO:21 and a TCR beta chain comprising an amino acid sequence set forth in SEQ ID NO:32;
j. a TCR alpha chain comprising an amino acid sequence set forth in SEQ ID NO:22 and a TCR beta chain comprising an amino acid sequence set forth in SEQ ID NO:33; or
k. a TCR alpha chain comprising an amino acid sequence set forth in SEQ ID NO:23 and a TCR beta chain comprising an amino acid sequence set forth in SEQ ID NO:34.

40. The cell of claim 39, said TCR comprising:
a. a TCR alpha chain comprising an amino acid sequence set forth in SEQ ID NO:35 and a TCR beta chain comprising an amino acid sequence set forth in SEQ ID NO:46;
b. a TCR alpha chain comprising an amino acid sequence set forth in SEQ ID NO:36 and a TCR beta chain comprising an amino acid sequence set forth in SEQ ID NO:47;
c. a TCR alpha chain comprising an amino acid sequence set forth in SEQ ID NO:37 and a TCR beta chain comprising an amino acid sequence set forth in SEQ ID NO:48;
d. a TCR alpha chain comprising an amino acid sequence set forth in SEQ ID NO:38 and a TCR beta chain comprising an amino acid sequence set forth in SEQ ID NO:49;
e. a TCR alpha chain comprising an amino acid sequence set forth in SEQ ID NO:39 and a TCR beta chain comprising an amino acid sequence set forth in SEQ ID NO:50;
f. a TCR alpha chain comprising an amino acid sequence set forth in SEQ ID NO:40 and a TCR beta chain comprising an amino acid sequence set forth in SEQ ID NO:51;
g. a TCR alpha chain comprising an amino acid sequence set forth in SEQ ID NO:41 and a TCR beta chain comprising an amino acid sequence set forth in SEQ ID NO: 52;
h. a TCR alpha chain comprising an amino acid sequence set forth in SEQ ID NO:42 and a TCR beta chain comprising an amino acid sequence set forth in SEQ ID NO:53;
i. a TCR alpha chain comprising an amino acid sequence set forth in SEQ ID NO:43 and a TCR beta chain comprising an amino acid sequence set forth in SEQ ID NO: 54;
j. a TCR alpha chain comprising an amino acid sequence set forth in SEQ ID NO:44 and a TCR beta chain comprising an amino acid sequence set forth in SEQ ID NO:55; or
k. a TCR alpha chain comprising an amino acid sequence set forth in SEQ ID NO:45 and a TCR beta chain comprising an amino acid sequence set forth in SEQ ID NO:56.

41. The cell of claim 39, wherein the cell further expresses a recombinant IL-12 or functional variant thereof.

42. A cell comprising an expression vector of claim 1.

43. The cell of claim 39, wherein the cell is a T cell.

44. The cell of claim 43, wherein the TCR binds the peptide of SEQ ID NO:1 or SEQ ID NO:2 in the context of HLA-A*02:01 and said binding leads to activation of IFNγ, TNFα, IL-12, or granzyme B production by said cell.

45. A pharmaceutical composition comprising a therapeutically effective amount of a cell of claim 39.

46. A method of making a cell of claim 39, comprising introducing into a cell an expression vector comprising a nucleic acid sequence encoding a TCR alpha chain and a TCR beta chain, wherein the TCR alpha chain and TCR beta chain are selected from the group consisting of:
a. a TCR alpha chain comprising an amino acid sequence set forth in SEQ ID NO:13 and a TCR beta chain comprising an amino acid sequence set forth in SEQ ID NO:24;
b. a TCR alpha chain comprising an amino acid sequence set forth in SEQ ID NO:14 and a TCR beta chain comprising an amino acid sequence set forth in SEQ ID NO:25;
c. a TCR alpha chain comprising an amino acid sequence set forth in SEQ ID NO:15 and a TCR beta chain comprising an amino acid sequence set forth in SEQ ID NO:26;
d. a TCR alpha chain comprising an amino acid sequence set forth in SEQ ID NO:16 and a TCR beta chain comprising an amino acid sequence set forth in SEQ ID NO:27;
e. a TCR alpha chain comprising an amino acid sequence set forth in SEQ ID NO:17 and a TCR beta chain comprising an amino acid sequence set forth in SEQ ID NO:28;
f. a TCR alpha chain comprising an amino acid sequence set forth in SEQ ID NO: 18 and a TCR beta chain comprising an amino acid sequence set forth in SEQ ID NO:29;
g. a TCR alpha chain comprising an amino acid sequence set forth in SEQ ID NO:19 and a TCR beta chain comprising an amino acid sequence set forth in SEQ ID NO:30;
h. a TCR alpha chain comprising an amino acid sequence set forth in SEQ ID NO:20 and a TCR beta chain comprising an amino acid sequence set forth in SEQ ID NO:31;
i. a TCR alpha chain comprising an amino acid sequence set forth in SEQ ID NO:21 and a TCR beta chain comprising an amino acid sequence set forth in SEQ ID NO:32;
j. a TCR alpha chain comprising an amino acid sequence set forth in SEQ ID NO:22 and a TCR beta chain comprising an amino acid sequence set forth in SEQ ID NO:33; or
k. a TCR alpha chain comprising an amino acid sequence set forth in SEQ ID NO:23 and a TCR beta chain comprising an amino acid sequence set forth in SEQ ID NO:34.

47. The method of claim 46, wherein the TCR alpha chain and TCR beta chain are selected from the group consisting of:
a. a TCR alpha chain comprising an amino acid sequence set forth in SEQ ID NO:35 and a TCR beta chain comprising an amino acid sequence set forth in SEQ ID NO:46;
b. a TCR alpha chain comprising an amino acid sequence set forth in SEQ ID NO:36 and a TCR beta chain comprising an amino acid sequence set forth in SEQ ID NO:47;
c. a TCR alpha chain comprising an amino acid sequence set forth in SEQ ID NO:37 and a TCR beta chain comprising an amino acid sequence set forth in SEQ ID NO:48;
d. a TCR alpha chain comprising an amino acid sequence set forth in SEQ ID NO:38 and a TCR beta chain comprising an amino acid sequence set forth in SEQ ID NO:49;
e. a TCR alpha chain comprising an amino acid sequence set forth in SEQ ID NO:39 and a TCR beta chain comprising an amino acid sequence set forth in SEQ ID NO:50;
f. a TCR alpha chain comprising an amino acid sequence set forth in SEQ ID NO:40 and a TCR beta chain comprising an amino acid sequence set forth in SEQ ID NO:51;
g. a TCR alpha chain comprising an amino acid sequence set forth in SEQ ID NO:41 and a TCR beta chain comprising an amino acid sequence set forth in SEQ ID NO:52;
h. a TCR alpha chain comprising an amino acid sequence set forth in SEQ ID NO:42 and a TCR beta chain comprising an amino acid sequence set forth in SEQ ID NO:53;
i. a TCR alpha chain comprising an amino acid sequence set forth in SEQ ID NO:43 and a TCR beta chain comprising an amino acid sequence set forth in SEQ ID NO: 54;
j. a TCR alpha chain comprising an amino acid sequence set forth in SEQ ID NO:44 and a TCR beta chain comprising an amino acid sequence set forth in SEQ ID NO:55; and
k. a TCR alpha chain comprising an amino acid sequence set forth in SEQ ID NO:45 and a TCR beta chain comprising an amino acid sequence set forth in SEQ ID NO:56.

48. The method of claim 46, wherein the expression vector further comprises a nucleic acid sequence encoding IL-12 or a functional variant thereof.

49. The method of claim 46, wherein the cell is a T cell.

50. The method of claim 49, wherein the T cell is a primary T cell.

51. The method of 50, wherein the primary T cell is isolated from a cancer patient.

52. A method of treating a MAGE-B2 or MAGE-A4 expressing cancer, said method comprising administering to a cancer patient a therapeutically effective amount of a cell of claim 39.

53. The method of claim 52, wherein the patient is tested prior to administration to determine the presence of a cancer expressing MAGE-B2 or MAGE-A4.

54. The method of claim 53, wherein a nucleic acid encoding MAGE-B2 or MAGE-A4 is detected.

55. The method of claim 53, wherein MAGE-B2 or MAGE-A4 protein or a MAGE-B2-derived or MAGE-A4-derived peptide is detected.

56. The method of any claim 52, wherein the patient is identified to carry the HLA-A*02:01 allele.

57. A method of treating a MAGE-B2 or MAGE-A4 expressing cancer, said method comprising administering to a cancer patient a pharmaceutical composition of claim 45.

58. A method of treating a MAGE-B2 or MAGE-A4 expressing cancer, said method comprising administering to a cancer patient a cell made by the method of claim 46.

59. A method of making a pharmaceutical composition of claim 45, comprising introducing into a cell an expression vector comprising a nucleic acid sequence encoding a TCR alpha chain and a TCR beta chain, wherein the TCR alpha chain and TCR beta chain are selected from the group consisting of:
a. a TCR alpha chain comprising an amino acid sequence set forth in SEQ ID NO:13 and a TCR beta chain comprising an amino acid sequence set forth in SEQ ID NO:24;
b. a TCR alpha chain comprising an amino acid sequence set forth in SEQ ID NO:14 and a TCR beta chain comprising an amino acid sequence set forth in SEQ ID NO:25;
c. a TCR alpha chain comprising an amino acid sequence set forth in SEQ ID NO:15 and a TCR beta chain comprising an amino acid sequence set forth in SEQ ID NO:26;
d. a TCR alpha chain comprising an amino acid sequence set forth in SEQ ID NO:16 and a TCR beta chain comprising an amino acid sequence set forth in SEQ ID NO:27;
e. a TCR alpha chain comprising an amino acid sequence set forth in SEQ ID NO:17 and a TCR beta chain comprising an amino acid sequence set forth in SEQ ID NO:28;
f. a TCR alpha chain comprising an amino acid sequence set forth in SEQ ID NO: 18 and a TCR beta chain comprising an amino acid sequence set forth in SEQ ID NO:29;
g. a TCR alpha chain comprising an amino acid sequence set forth in SEQ ID NO:19 and a TCR beta chain comprising an amino acid sequence set forth in SEQ ID NO:30;
h. a TCR alpha chain comprising an amino acid sequence set forth in SEQ ID NO:20 and a TCR beta chain comprising an amino acid sequence set forth in SEQ ID NO:31;
i. a TCR alpha chain comprising an amino acid sequence set forth in SEQ ID NO:21 and a TCR beta chain comprising an amino acid sequence set forth in SEQ ID NO:32;
j. a TCR alpha chain comprising an amino acid sequence set forth in SEQ ID NO:22 and a TCR beta chain comprising an amino acid sequence set forth in SEQ ID NO:33; or
k. a TCR alpha chain comprising an amino acid sequence set forth in SEQ ID NO:23 and a TCR beta chain comprising an amino acid sequence set forth in SEQ ID NO:34.

60. The method of claim 59, wherein the TCR alpha chain and TCR beta chain are selected from the group consisting of:
a. a TCR alpha chain comprising an amino acid sequence set forth in SEQ ID NO:35 and a TCR beta chain comprising an amino acid sequence set forth in SEQ ID NO:46;
b. a TCR alpha chain comprising an amino acid sequence set forth in SEQ ID NO:36 and a TCR beta chain comprising an amino acid sequence set forth in SEQ ID NO:47;
c. a TCR alpha chain comprising an amino acid sequence set forth in SEQ ID NO:37 and a TCR beta chain comprising an amino acid sequence set forth in SEQ ID NO:48;
d. a TCR alpha chain comprising an amino acid sequence set forth in SEQ ID NO:38 and a TCR beta chain comprising an amino acid sequence set forth in SEQ ID NO:49;
e. a TCR alpha chain comprising an amino acid sequence set forth in SEQ ID NO:39 and a TCR beta chain comprising an amino acid sequence set forth in SEQ ID NO:50;
f. a TCR alpha chain comprising an amino acid sequence set forth in SEQ ID NO:40 and a TCR beta chain comprising an amino acid sequence set forth in SEQ ID NO:51;
g. a TCR alpha chain comprising an amino acid sequence set forth in SEQ ID NO:41 and a TCR beta chain comprising an amino acid sequence set forth in SEQ ID NO: 52;
h. a TCR alpha chain comprising an amino acid sequence set forth in SEQ ID NO:42 and a TCR beta chain comprising an amino acid sequence set forth in SEQ ID NO:53;
i. a TCR alpha chain comprising an amino acid sequence set forth in SEQ ID NO:43 and a TCR beta chain comprising an amino acid sequence set forth in SEQ ID NO: 54;
j. a TCR alpha chain comprising an amino acid sequence set forth in SEQ ID NO:44 and a TCR beta chain comprising an amino acid sequence set forth in SEQ ID NO:55; and
k. a TCR alpha chain comprising an amino acid sequence set forth in SEQ ID NO:45 and a TCR beta chain comprising an amino acid sequence set forth in SEQ ID NO:56.


SEQUENCE LISTING (78 sequences)

SEQ ID NO: 1 - protein, 10 aa, Homo sapiens
GVYDGEEHSV

SEQ ID NO: 2 - protein, 10 aa, Homo sapiens
GVYDGREHTV

SEQ ID NO: 3 - protein, 10 aa, Homo sapiens
GLYDGREHSV

SEQ ID NO: 4 - protein, 10 aa, Homo sapiens
GVIDGHIYAV

SEQ ID NO: 5 - protein, 10 aa, Homo sapiens
GLSDGEWQLV

SEQ ID NO: 6 - protein, 10 aa, Homo sapiens
GVMAGDIYSV

SEQ ID NO: 7 - protein, 10 aa, Homo sapiens
GLYDGPVHEV

SEQ ID NO: 8 - protein, 10 aa, Homo sapiens
GVYGGSVHEA

SEQ ID NO: 9 - protein, 10 aa, Homo sapiens
FVYDEPGHAV

SEQ ID NO: 10 - protein, 10 aa, Homo sapiens
TVYGGYLCSV

SEQ ID NO: 11 - protein, 10 aa, Homo sapiens
YTYDEAIHSV

SEQ ID NO: 12 - protein, 10 aa, Homo sapiens
FIVDGVEVSV

SEQ ID NO: 13 - protein, 13 aa, Homo sapiens
CAAMKTSYDKVIF

SEQ ID NO: 14 - protein, 18 aa, Homo sapiens
CAVNIPFSNSGGYQKVTF

SEQ ID NO: 15 - protein, 16 aa, Homo sapiens
CALSVLRMDSSYKLIF

SEQ ID NO: 16 - protein, 12 aa, Homo sapiens
CVVSLGTDKLIF

SEQ ID NO: 17 - protein, 10 aa, Homo sapiens
CAPGGNQFYF

SEQ ID NO: 18 - protein, 11 aa, Homo sapiens
CAFFNAGKSTF

SEQ ID NO: 19 - protein, 14 aa, Homo sapiens
CAVRRLGGYQKVTF

SEQ ID NO: 20 - protein, 14 aa, Homo sapiens
CAMRGPTSYGKLTF

SEQ ID NO: 21 - protein, 9 aa, Homo sapiens
CVVSSDMRF

SEQ ID NO: 22 - protein, 11 aa, Homo sapiens
CAVRDNARLMF

SEQ ID NO: 23 - protein, 14 aa, Homo sapiens
CAEKSITSYDKVIF

SEQ ID NO: 24 - protein, 14 aa, Homo sapiens
CASSQGQGGYGYTF

SEQ ID NO: 25 - protein, 15 aa, Homo sapiens
CASRHPGQYNQPQHF

SEQ ID NO: 26 - protein, 14 aa, Homo sapiens
CASSLQGAGQPQHF

SEQ ID NO: 27 - protein, 14 aa, Homo sapiens
CATSAQGNYNEQFF

SEQ ID NO: 28 - protein, 12 aa, Homo sapiens
CASSGSNQPQHF

SEQ ID NO: 29 - protein, 14 aa, Homo sapiens
CASTVGGGPYGYTF

SEQ ID NO: 30 - protein, 16 aa, Homo sapiens
CASSLVTGSSYNEQFF

SEQ ID NO: 31 - protein, 14 aa, Homo sapiens
CATSPTTDNQPQHF

SEQ ID NO: 32 - protein, 12 aa, Homo sapiens
CASSYGGDEQYF

SEQ ID NO: 33 - protein, 13 aa, Homo sapiens
CSVGPSGHTGYTF

SEQ ID NO: 34 - protein, 13 aa, Homo sapiens
CASTRRGTYGYTF

SEQ ID NO: 35 - protein, 253 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAAMKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 257 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNIPFSNSGGYQKVTFGTGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 37 - protein, 258 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSVLRMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 252 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVVSLGTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 247 aa, Homo sapiens
KNEVEQSPQNLTAQEGEFITINCSYSVGISALHWLQQHPGGGIVSLFMLSSGKKKHGRLIATINIQEKHSSLHITASHPRDSAVYICAPGGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 253 aa, Homo sapiens
AQTVTQSQPEMSVQEAETVTLSCTYDTSENNYYLFWYKQPPSRQMILVIRQEAYKQQNATENRFSVNFQKAAKSFSLKISDSQLGDTAMYFCAFFNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 41 - protein, 252 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRRLGGYQKVTFGTGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 256 aa, Homo sapiens
AQKITQTQPGMFVQEKEAVTLDCTYDTSDQSYGLFWYKQPSSGEMIFLIYQGSYDEQNATEGRYSLNFQKARKSANLVISASQLGDSAMYFCAMRGPTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 43 - protein, 249 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVVSSDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 252 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 45 - protein, 253 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEKSITSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 291 aa, Homo sapiens
GEEVAQTPKHLVRGEGQKAKLYCAPIKGHSYVFWYQQVLKNEFKFLISFQNENVFDETGMPKERFSAKCLPNSPCSLEIQATKLEDSAVYFCASSQGQGGYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 291 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASRHPGQYNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 48 - protein, 290 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSLQGAGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 292 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSAQGNYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 50 - protein, 288 aa, Homo sapiens
DAGITQSPRYKITETGRQVTLMCHQTWSHSYMFWYRQDLGHGLRLIYYSAAADITDKGEVPDGYVVSRSKTENFPLTLESATRSQTSVYFCASSGSNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 51 - protein, 291 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASTVGGGPYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 52 - protein, 295 aa, Homo sapiens
GAGVSQSPSNKVTEKGKDVELRCDPISGHTALYWYRQRLGQGLEFLIYFQGNSAPDKSGLPSDRFSAERTGESVSTLTIQRTQQEDSAVYLCASSLVTGSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 53 - protein, 290 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSPTTDNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 54 - protein, 290 aa, Homo sapiens
NAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYGGDEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 55 - protein, 291 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGPSGHTGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 56 - protein, 289 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASTRRGTYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 57 - protein, 272 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAAMKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 278 aa, Homo sapiens
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNIPFSNSGGYQKVTFGTGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 59 - protein, 278 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSVLRMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 273 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSLGTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 61 - protein, 269 aa, Homo sapiens
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAPGGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 273 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFFNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 63 - protein, 269 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRRLGGYQKVTFGTGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 276 aa, Homo sapiens
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMRGPTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 65 - protein, 270 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSSDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 271 aa, Homo sapiens
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 67 - protein, 274 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEKSITSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 310 aa, Homo sapiens
MSPIFTCITILCLLAAGSPGEEVAQTPKHLVRGEGQKAKLYCAPIKGHSYVFWYQQVLKNEFKFLISFQNENVFDETGMPKERFSAKCLPNSPCSLEIQATKLEDSAVYFCASSQGQGGYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 69 - protein, 310 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASRHPGQYNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 70 - protein, 309 aa, Homo sapiens
MSLGLLCCAAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSLQGAGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 311 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSAQGNYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 72 - protein, 307 aa, Homo sapiens
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSGSNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 73 - protein, 310 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASTVGGGPYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 74 - protein, 314 aa, Homo sapiens
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSLVTGSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 75 - protein, 309 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSPTTDNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 76 - protein, 309 aa, Homo sapiens
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYGGDEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 77 - protein, 306 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGPSGHTGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 78 - protein, 308 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASTRRGTYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF